(19) United States
(12) Patent Application Publication
(10) Pub. No.: US 2024/0131121 A1
(43) Pub. Date: Apr. 25, 2024

(54) NOVEL OMNI-50 CRISPR NUCLEASE

(71) Applicant: EmendoBio Inc., Wilmington, DE (US)

(72) Inventors: David Baram, Tel Aviv (IL); Lior Izhar, Tel Aviv (IL); Asael Herman, Ness-Ziona (IL); Liat Rockah, Rishon LeZion (IL); Nadav Marbach-Bar, Rehovot (IL); Nurit Meron, Ramat Gan (IL); Joseph Georgeson, Rehovot (IL)

(73) Assignee: EmendoBio Inc., Wilmington, DE (US)

(21) Appl. No.: 18/296,798

(22) Filed: Apr. 5, 2023

Related U.S. Application Data
(62) Division of application No. 17/594,761, filed on Oct. 28, 2021, now Pat. No. 11,666,641.
(60) Provisional application No. 62/991,285, filed on Mar. 18, 2020.
(60) Provisional application No. 62/959,672, filed on Jan. 10, 2020.
(60) Provisional application No. 62/931,630, filed on Nov. 6, 2019.
(60) Provisional application No. 62/897,806, filed on Sep. 9, 2019.
(60) Provisional application No. 62/841,046, filed on Apr. 30, 2019.

(51) Int. Cl. A61K 38/46; A61K 31/7105; C12N 9/22; C12N 15/11; C12N 15/90

(52) U.S. Cl. CPC A61K 38/465; A61K 31/7105; C12N 9/22; C12N 15/11; C12N 15/907; C12N 2310/20; C12N 2800/80

(57) ABSTRACT

The present invention provides a non-naturally occurring composition comprising a CRISPR nuclease comprising a sequence having at least 95% identity to the amino acid sequence of SEQ ID NO: 3 or a nucleic acid molecule comprising a sequence encoding the CRISPR nuclease.

This application claims the benefit of U.S. Provisional Application Nos. 62/991,285 filed Mar. 18, 2020, 62/959,672 filed Jan. 10, 2020, 62/931,630 filed Nov. 6, 2019, 62/897,806 filed Sep. 9, 2019, and 62/841,046 filed Apr. 30, 2019, the contents of which are hereby incorporated by reference.

Throughout this application, various publications are referenced, including referenced in parenthesis. The disclosures of all publications mentioned in this application in their entireties are hereby incorporated by reference into this application in order to provide additional description of the art to which this invention pertains and of the features in the art which can be employed with this invention.

### REFERENCE TO SEQUENCE LISTING

This application incorporates-by-reference nucleotide sequences which are present in the file named “200430 91116-A-PCT_SequenceListing_AWG.txt”, which is 186 kilobytes in size, and which was created on Apr. 29, 2020 in the IBM-PC machine format, having an operating system compatibility with MS-Windows, which is contained in the text file filed Apr. 30, 2020 as part of this application.

### FIELD OF THE INVENTION

The present invention is directed to, inter alia, composition and methods for genome editing.

### BACKGROUND OF THE INVENTION

The Clustered Regularly Interspaced Short Palindromic Repeat (CRISPR) systems of bacterial and archaeal adaptive immunity show extreme diversity of protein composition and genomic loci architecture. The CRISPR systems have become important tools for research and genome engineering. Nevertheless, many details of CRISPR systems have not been determined and the applicability of CRISPR nucleases may be limited by sequence specificity requirements, expression, or delivery challenges. Different CRISPR nucleases have diverse characteristics such as: size, PAM site, on target activity, specificity, cleavage pattern (e.g. blunt, staggered ends), and prominent pattern of indel formation following cleavage. Different sets of characteristics may be useful for different applications. For example, some CRISPR nucleases may be able to target particular genomic loci that other CRISPR nucleases cannot due to limitations of the PAM site. In addition, some CRISPR nucleases currently in use exhibit pre-immunity, which may limit in vivo applicability. See Charlesworth et al., Nature Medicine (2019) and Wagner et al., Nature Medicine (2019). Accordingly, discovery, engineering, and improvement of novel CRISPR nucleases is of importance.

### SUMMARY OF THE INVENTION

Disclosed herein are compositions and methods that may be utilized for genomic engineering, epigenomic engineering, genome targeting, genome editing of cells, and/or in vitro diagnostics.

The disclosed compositions may be utilized for modifying genomic DNA sequences. As used herein, genomic DNA refers to linear and/or chromosomal DNA and/or plasmid or other extrachromosomal DNA sequences present in the cell or cells of interest. In some embodiments, the cell of interest is a eukaryotic cell. In some embodiments, the cell of interest is a prokaryotic cell. In some embodiments, the methods produce double-stranded breaks (DSBs) at pre-determined target sites in a genomic DNA sequence, resulting in mutation, insertion, and/or deletion of a DNA sequence at the target site(s) in a genome.

Accordingly, in some embodiments, the compositions comprise a Clustered Regularly Interspaced Short Palindromic Repeat (CRISPR) nucleases. In some embodiments, the CRISPR nuclease is a CRISPR-associated protein.

In some embodiments, the compositions comprise a Clustered Regularly Interspaced Short Palindromic Repeats (CRISPR) nuclease having 100%, 99%, 98%, 97%, 96%, 95%, 94%, 93%, 92%, 91%, 90%, 85% identity to a CRISPR nuclease derived from Ezakiella peruensis strain M6.X2. Each possibility represents a separate embodiment.

### OMNI-50 Nuclease

Embodiments of the present invention provide for a CRISPR nuclease designated as an “OMNI-50” nuclease, as provided in Table 1.

This invention provides a method of modifying a nucleotide sequence at a target site in the genome of a mammalian cell comprising introducing into the cell (i) a composition comprising a CRISPR nuclease having at least 95% identity to an amino acid sequence of SEQ ID NO: 3 or a nucleic acid molecule comprising a sequence encoding a CRISPR nuclease which sequence has at least 95% identity to the nucleic acid sequence of SEQ ID NOs: 12 or 13 and (ii) a DNA-targeting RNA molecule, or a DNA polynucleotide encoding a DNA-targeting RNA molecule, comprising a nucleotide sequence that is complementary to a sequence in the target DNA.

This invention also provides a non-naturally occurring composition comprising a CRISPR associated system comprising:
a) one or more RNA molecules comprising a guide sequence portion linked to a direct repeat sequence, wherein the guide sequence is capable of hybridizing with a target sequence, or one or more nucleotide sequences encoding the one or more RNA molecules; and
b) a CRISPR nuclease comprising an amino acid sequence having at least 95% identity to the amino acid sequence of SEQ ID NO: 3 or a nucleic acid molecule comprising a sequence encoding the CRISPR nuclease; and
wherein the one or more RNA molecules hybridize to the target sequence, wherein the target sequence is 3′ of a Protospacer Adjacent Motif (PAM), and the one or more RNA molecules form a complex with the RNA-guided nuclease.

This invention also provides a non-naturally occurring composition comprising:
a) a CRISPR nuclease comprising a sequence having at least 95% identity to the amino acid sequence of SEQ ID NO: 3 or a nucleic acid molecule comprising a sequence encoding the CRISPR nuclease; and
b) one or more RNA molecules, or one or more DNA polynucleotide encoding the one or more RNA molecules, comprising at least one of:
   i) a nuclease-binding RNA nucleotide sequence capable of interacting with/binding to the CRISPR nuclease; and
   ii) a DNA-targeting RNA nucleotide sequence comprising a sequence complementary to a sequence in a target DNA sequence,
wherein the CRISPR nuclease is capable of complexing with the one or more RNA molecules to form a complex capable of hybridizing with the target DNA sequence.

### BRIEF DESCRIPTION OF THE DRAWINGS

FIG. 1A: An example of the predicted secondary structures of the full duplex RNA elements (crRNA:tracrRNA chimera) used for identification of possible elements in the design of sgRNAs for each nuclease. FIGS. 1B-1C: An example of the variation in structure between regions of two different sgRNAs, V1 (FIG. 1B) and V2 (FIG. 1C), designed for use with a single nuclease. By shortening the duplex at the upper stem at different locations, the crRNA and tracrRNA were connected with tetra-loop ‘gaaa’, generating sgRNA scaffolds.

FIG. 2A: A condensed 4N window library of all possible PAM locations along an 8 bp sequence for OMNI-50 sgRNA V1 in a cell-free in vitro TXTL system. Sequence motifs generated for PAM sites based on depletion assay results. Activity estimated based on the average of the two most depleted sequences and was calculated as: 1— Depletion score. FIG. 2B: The sequence motifs generated for all possible PAM locations along an 8 bp sequence for the OMNI-50 sgRNA V2.

FIG. 3: Expression of OMNI-50 in mammalian cells. OMNI-50 or SpCas9 nuclease were transiently transfected in Hek293T cells. Cells were harvested and lysed at 72 h, and the lysates were used to test OMNI-50 expression in the mammalian cells by western blot using an antibody against the HA-tag. SpCas9-HA was transfected in the same manner served as a positive control. GAPDH was used to normalize loading quantities.

FIG. 4A: Intrinsic fidelity in human cells. OMNI-50 or SpCas9 nuclease were expressed in mammalian cell system by DNA transfection together with sgRNA expressing plasmid. Cell lysates were used for site specific genomic DNA amplification and NGS. The percentage of Indels was measured and analyzed as described in section vii, target vs off-target editing in HeLa cell line using ELANEg35 OMNI-50 or ELANEg62 OMNI-50. In both cases the genomic On and Off target sequences are noted below the chart, PAM sequence in underline. Each experiment represents 3 independent repeats. FIG. 4B: RNP introduction of OMNI-50 or SpCas9 targeting ELANEg35 was followed by lysis, site specific DNA amplification and NGS. Editing level of both On and Off target sequences is shown in a second system.

FIG. 5A-FIG. 5D: OMNI-50 activity Assay as RNP. OMNI-50 nuclease was over-expressed and purified. The purified protein was complexed with synthetic sgRNA to form RNPs. For the in-vitro assays (FIG. 5A and FIG. 5B) RNPs were incubated with a linear DNA template containing the corresponding target and PAM sequences (listed in Table 5). Activity was verified by cleavage of the linear template. For the in-vivo assays (FIG. 5C and FIG. 5D), U2OS cells were electroporated with RNPs and activity was determined by measurement of indel frequency by NGS. FIG. 5A: Activity assay of OMNI-50 RNP with different spacer lengths (17-23 bps) of guide 35 (Table 10). FIG. 5B: Decreasing amounts of RNPs (4 pmol, 2 pmol, 1.2 pmol, 0.6 pmol and 0.2 pmol) with spacer lengths 20-23 nts were incubated with 100 ng DNA target template. FIG. 5C: Activity assay for OMNI-50 as RNP in U2OS cells: RNPs with spacer lengths 17-23 bps were electroporated into U2OS cell line and editing levels (indels) measured by NGS. FIG. 5D: Activity assay for OMNI-50 as RNP in U2OS cells: RNPs with ELANE g35 sgRNA V1-V4 were electroporated into U2OS cell line and editing levels (indels) measured by NGS.

FIG. 6: Activity assay for OMNI-50 as RNP in iPSCs. RNPs with spacer lengths 17-23 nts (Table 10) were electroporated into an iPSC cell line and editing levels (indels) were measured by NGS.

FIG. 7. OMNI-50 nuclease activity in an endogenous mammalian cellular context. OMNI-50 nuclease was expressed in mammalian cell system by DNA transfection together with sgRNA expressing plasmid. Cell lysates were used for site specific genomic DNA amplification and NGS. The percentage of indels was measured and analyzed to determine the editing level. Cells transfected with the OMNI-50 nuclease without a guide RNA served as a negative control for comparison and background determination. Editing levels in different genomic locations are shown.

FIG. 8: Schematic representation of the OMNI-50 nuclease. The OMNI-50 nuclease comprises several functional domains, represented in the schematic as Domains A-F. Domain A comprises three subdomains, A1, A2, and A3, and is represented in the schematic as white boxes. Domain B is represented in the schematic with horizontal stripes. Domain C comprises three subdomains, C1, C2, and C3, and is represented in the schematic as a lightly shaded box. Domain B is represented in the schematic with diagonal stripes. Domain E is represented in the schematic as a dark shaded box. Domain F is represented in the schematic as a dotted box.

### DETAILED DESCRIPTION

According to some aspects of the invention, the disclosed compositions comprise a Clustered Regularly Interspaced Short Palindromic Repeats (CRISPR) nuclease and/or a nucleic acid molecule comprising a sequence encoding the same.

In some embodiments, the CRISPR nuclease comprises an amino acid sequence having at least 100%, 99%, 98%, 97%, 96%, 95%, 94%, 93%, 92%, 91%, 90%, 89%, 88%, 87%, 86%, 85%, 84%, 83%, or 82% amino acid sequence identity to a CRISPR nuclease as set forth as SEQ ID NO: 3. In an embodiment the sequence encoding the CRISPR nuclease has at least 95% identity to a nucleic acid sequence selected from the group consisting of SEQ ID NOs: 11-13.

In some embodiments, the CRISPR nuclease comprises an amino acid sequence having at least 100%, 99%, 98%, 97%, 96%, 95%, 94%, 93%, 92%, 91%, 90%, 89%, 88%, 87%, 86%, 85%, 84%, 83%, 82%, 81%, 80%, 75% amino acid sequence identity to a CRISPR nucleases derived from Ezakiella peruensis strain M6.X2. Each possibility represents a separate embodiment.

According to some aspects of the invention, the disclosed compositions comprise DNA constructs or a vector system comprising nucleotide sequences that encode the CRISPR nuclease or variant CRISPR nuclease. In some embodiments, the nucleotide sequence that encode the CRISPR nuclease or variant CRISPR nuclease is operably linked to a promoter that is operable in the cells of interest. In some embodiments, the cell of interest is a eukaryotic cell. In some embodiments the cell of interest is a mammalian cell. In some embodiments, the nucleic acid sequence encoding the engineered CRISPR nuclease is codon optimized for use in cells from a particular organism. In some embodiments, the nucleic acid sequence encoding the nuclease is codon optimized for E. Coli. In some embodiments, the nucleic acid sequence encoding the nuclease is codon optimized for eukaryotic cells. In some embodiments, the nucleic acid sequence encoding the nuclease is codon optimized for mammalian cells.

In some embodiments, the composition comprises a recombinant nucleic acid, comprising a heterologous promoter operably linked to a polynucleotide encoding a CRISPR enzyme having at least 100%, 99%, 98%, 97%, 96%, 95%, 94%, 93%, 92%, 91%, 90% identity to SEQ ID NO: 3. Each possibility represents a separate embodiment.

In an embodiment of the composition, the CRISPR nuclease has at least 75%, 80%, 85, 90%, 95%, or 97% identity to the amino acid sequence as set forth in SEQ ID NO: 3 or the sequence encoding the CRISPR nuclease has at least a 75%, 80%, 85, 90%, 95%, or 97% sequence identity to a nucleotide sequence selected from the group consisting of SEQ ID NOs: 11, 12, and 13.

According to some embodiments, there is provided an engineered or non-naturally occurring composition comprising a CRISPR nuclease comprising a sequence having at least 100%, 99%, 98%, 97%, 96%, 95%, 94%, 93%, 92%, 91%, 90%, 85%, 80% identity to the amino acid sequence of SEQ ID NO: 3 or a nucleic acid molecule comprising a sequence encoding the CRISPR nuclease. Each possibility represents a separate embodiment.

In an embodiment, the CRISPR nuclease is engineered or non-naturally occurring. The CRISPR nuclease may also be recombinant. Such CRISPR nucleases are produced using laboratory methods (molecular cloning) to bring together genetic material from multiple sources, creating sequences that would not otherwise be found in biological organisms.

In an embodiment, the CRISPR nuclease of the invention exhibits increased specificity to a target site compared to a SpCas9 nuclease when complexed with the one or more RNA molecules.

In an embodiment, the complex of the CRISPR nuclease of the invention and one or more RNA molecules exhibits at least maintained on-target editing activity of the target site and reduced off-target activity compared to SpCas9 nuclease.

In an embodiment, the CRISPR nuclease further comprises an RNA-binding portion capable of interacting with a DNA-targeting RNA molecule (gRNA) and an activity portion that exhibits site-directed enzymatic activity.

In an embodiment, the composition further comprises a DNA-targeting RNA molecule or a DNA polynucleotide encoding a DNA-targeting RNA molecule, wherein the DNA-targeting RNA molecule comprises a nucleotide sequence that is complementary to a sequence in a target region, wherein the DNA-targeting RNA molecule and the CRISPR nuclease do not naturally occur together.

In an embodiment, the DNA-targeting RNA molecule comprises a crRNA repeat sequence which comprises the sequence GUUUGAGAG.

In an embodiment, the DNA-targeting RNA molecule comprises a tracrRNA sequence which comprises one or more sequences selected from SEQ ID NOs: 41-43 and SEQ ID NOs: 149-154.

In an embodiment, the DNA-targeting RNA molecule further comprises a nucleotide sequence that can form a complex with a CRISPR nuclease.

This invention also provides a non-naturally occurring composition comprising a CRISPR associated system comprising:
a) one or more RNA molecules comprising a guide sequence portion linked to a direct repeat sequence, wherein the guide sequence is capable of hybridizing with a target sequence, or one or more nucleotide sequences encoding the one or more RNA molecules; and
b) a CRISPR nuclease comprising an amino acid sequence having at least 95% identity to the amino acid sequence of SEQ ID NO: 3 or a nucleic acid molecule comprising a sequence encoding the CRISPR nuclease;
wherein the one or more RNA molecules hybridize to the target sequence, wherein the target sequence is 3′ of a Protospacer Adjacent Motif (PAM), and the one or more RNA molecules form a complex with the RNA-guided nuclease.

In an embodiment, the composition further comprises an RNA molecule comprising a nucleotide sequence that can form a complex with a CRISPR nuclease (tracrRNA) or a DNA polynucleotide comprising a sequence encoding an RNA molecule that can form a complex with the CRISPR nuclease.

In an embodiment, the composition further comprises a donor template for homology directed repair (HDR).

In an embodiment, the composition is capable of editing the target region in the genome of a cell.

In an embodiment of the composition the CRISPR nuclease has at least 100%, 99%, 98%, 97%, 96%, 95%, 94%, 93%, 92%, 91%, 90%, 85%, 80% identity to SEQ ID NO: 3, and the nucleotide sequence that can form a complex with the CRISPR nuclease in the DNA-targeting RNA molecule comprises a sequence selected from SEQ ID NOs: 37-45, 87-88, 149-154, and GUUUGAGAG.

According to some embodiments, there is provided a non-naturally occurring composition comprising:
(a) a CRISPR nuclease, or a polynucleotide encoding the CRISPR nuclease, comprising:
   an RNA-binding portion; and
   an activity portion that exhibits site-directed enzymatic activity, wherein the CRISPR nuclease has at least 100%, 99%, 98%, 97%, 96%, 95%, 94%, 93%, 92%, 91%, 90%, 85%, 80% identity to SEQ ID NO: 3; and
(b) one or more RNA molecules or a DNA polynucleotide encoding the one or more RNA molecules comprising:
   i) a DNA-targeting RNA sequence, comprising a nucleotide sequence that is complementary to a sequence in a target DNA sequence; and
   ii) a protein-binding RNA sequence, capable of interacting with the RNA-binding portion of the CRISPR nuclease,
wherein the DNA targeting RNA sequence and the CRISPR nuclease do not naturally occur together. Each possibility represents a separate embodiment.

In some embodiments, there is provided a single RNA molecule comprising the DNA-targeting RNA sequence and the protein-binding RNA sequence, wherein the RNA molecule can form a complex with the CRISPR nuclease and serve as the DNA targeting module. In some embodiments, the RNA molecule has a length of up to 1000 bases, 900 bases, 800 bases, 700 bases, 600 bases, 500 bases, 400 bases, 300 bases, 200 bases, 100 bases, 50 bases. Each possibility represents a separate embodiment. In some embodiments, a first RNA molecule comprising the DNA-targeting RNA sequence and a second RNA molecule comprising the protein-binding RNA sequence interact by base pairing or alternatively fused together to form one or more RNA molecules that complex with the CRISPR nuclease and serve as the DNA targeting module.

In some embodiments, the CRISPR nuclease has at least 100%, 99%, 98%, 97%, 96%, 95%, 94%, 93%, 92%, 91%, 90%, 85%, 80% identity to SEQ ID NO: 3, and the RNA molecule comprises a sequence selected from SEQ ID NOs: 37-45, 87-88, 149-154 and GUUUGAGAG.

This invention also provides a non-naturally occurring composition comprising:
a) a CRISPR nuclease comprising a sequence having at least 95% identity to the amino acid sequence of SEQ ID NO: 3 or a nucleic acid molecule comprising a sequence encoding the CRISPR nuclease; and
b) one or more RNA molecules, or one or more DNA polynucleotide encoding the one or more RNA molecules, comprising at least one of:
   i) a nuclease-binding RNA nucleotide sequence capable of interacting with/binding to the CRISPR nuclease; and
   ii) a DNA-targeting RNA nucleotide sequence comprising a sequence complementary to a sequence in a target DNA sequence,
wherein the CRISPR nuclease is capable of complexing with the one or more RNA molecules to form a complex capable of hybridizing with the target DNA sequence.

In an embodiment, the CRISPR nuclease and the one or more RNA molecules form a CRISPR complex that is capable of binding to the target DNA sequence to effect cleavage of the target DNA sequence.

In an embodiment, the CRISPR nuclease and at least one of the one or more RNA molecules do not naturally occur together.

In an embodiment:
a) the CRISPR nuclease comprises an RNA-binding portion and an activity portion that exhibits site-directed enzymatic activity;
b) the DNA-targeting RNA nucleotide sequence comprises a nucleotide sequence that is complementary to a sequence in a target DNA sequence; and
c) the nuclease-binding RNA nucleotide sequence comprises a sequence that interacts with the RNA-binding portion of the CRISPR nuclease.

In an embodiment, the nuclease-binding RNA nucleotide sequence and the DNA-targeting RNA nucleotide sequence are on a single guide RNA molecule (sgRNA), wherein the sgRNA molecule can form a complex with the CRISPR nuclease and serve as the DNA targeting module.

In an embodiment, the nuclease-binding RNA nucleotide sequence is on a first RNA molecule and the DNA-targeting RNA nucleotide sequence is on a single guide RNA molecule, and wherein the first and second RNA sequence interact by base-pairing or are fused together to form one or more RNA molecules or sgRNA that complex with the CRISPR nuclease and serve as the targeting module.

In an embodiment, the sgRNA has a length of up to 1000 bases, 900 bases, 800 bases, 700 bases, 600 bases, 500 bases, 400 bases, 300 bases, 200 bases, 100 bases, 50 bases.

In an embodiment, the composition further comprises a donor template for homology directed repair (HDR).

In some embodiments, (a) the CRISPR nuclease has at least 100%, 99%, 98%, 97%, 96%, 95%, 94%, 93%, 92%, 91%, 90%, 85%, 80% identity to SEQ ID NO: 3, or (b) the nucleic acid molecule comprising a sequence encoding the CRISPR nuclease comprises a sequence of at least a 95% sequence identity to the nucleic acid sequence as set forth in SEQ ID NO: 11, 12, or 13, and the PAM is NGG. Non-limiting examples of suitable PAM sequences include: GGG, AGG, and TGG. In this embodiment, the nucleotide sequence that can form a complex with the CRISPR nuclease in the DNA-targeting RNA molecule comprises a sequence selected from SEQ ID NOs: 37-45, 87-88, 149-154 and GUUUGAGAG.

In some embodiments, the CRISPR nuclease utilizes a PAM having a sequence of NAG or NGA.

In an embodiment, the CRISPR nuclease comprises 1-10, 10-20, 20-30, 30-40, 40-50, 50-60, 60-70, 70-80, 80-90, 90-100, 100-110, 110-120, 120-130, 130-140, or 140-150 amino acid substitutions, deletions, and/or insertions compared to the amino acid sequence of the wild-type of the CRISPR nuclease.

In an embodiment, the CRISPR nuclease exhibits at least 2%, 5%, 7% 10%, 15%, 20%, 25%, 30%, or 35% increased specificity compared the wild-type of the CRISPR nuclease.

In an embodiment, the CRISPR nuclease exhibits at least 2%, 5%, 7% 10%, 15%, 20%, 25%, 30%, or 35% increased activity compared the wild-type of the CRISPR nuclease.

In an embodiment, the CRISPR nuclease has altered PAM specificity compared to the wild-type of the CRISPR nuclease.

In an embodiment, the CRISPR nuclease is non-naturally occurring.

In an embodiment, the CRISPR nuclease is engineered and comprises unnatural or synthetic amino acids.

In an embodiment, the CRISPR nuclease is engineered and comprises one or more of a nuclear localization sequences (NLS), cell penetrating peptide sequences, and/or affinity tags.

In an embodiment, the CRISPR nuclease comprises one or more nuclear localization sequences of sufficient strength to drive accumulation of a CRISPR complex comprising the CRISPR nuclease in a detectable amount in the nucleus of a eukaryotic cell.

This invention also provides a method of modifying a nucleotide sequence at a target site in a cell-free system or the genome of a cell comprising introducing into the cell any of the compositions of the invention.

In an embodiment, the cell is a eukaryotic cell.

In another embodiment, the cell is a prokaryotic cell.

In some embodiments, the one or more RNA molecules further comprises an RNA sequence comprising a nucleotide molecule that can form a complex with the RNA nuclease (tracrRNA) or a DNA polynucleotide encoding an RNA molecule comprising a nucleotide sequence that can form a complex with the CRISPR nuclease.

In an embodiment, the CRISPR nuclease comprises 1, 2, 3, 4, 5, 6, 7, 8, 9, 10, or more NLSs at or near the amino-terminus, 1, 2, 3, 4, 5, 6, 7, 8, 9, 10, or more NLSs at or near carboxy-terminus, or a combination of 1, 2, 3, 4, 5, 6, 7, 8, 9, 10, or more NLSs at or near the amino-terminus and 1, 2, 3, 4, 5, 6, 7, 8, 9, 10, or more NLSs at or near carboxy-terminus. In an embodiment 1-4 NLSs are fused with the CRISPR nuclease. In an embodiment, an NLS is located within the open-reading frame (ORF) of the CRISPR nuclease.

Methods of fusing an NLS at or near the amino-terminus, at or near carboxy-terminus, or within the ORF of an expressed protein are well known in the art. As an example, to fuse an NLS to the amino-terminus of a CRISPR nuclease, the nucleic acid sequence of the NLS is placed immediately after the start codon of the CRISPR nuclease on the nucleic acid encoding the NLS-fused CRISPR nuclease. Conversely, to fuse an NLS to the carboxy-terminus of a CRISPR nuclease the nucleic acid sequence of the NLS is placed after the codon encoding the last amino acid of the CRISPR nuclease and before the stop codon.

Any combination of NLSs, cell penetrating peptide sequences, and/or affinity tags at any position along the ORF of the CRISPR nuclease is contemplated in this invention.

The amino acid sequences and nucleic acid sequences of the CRISPR nucleases provided herein may include NLS and/or TAGs inserted so as to interrupt the contiguous amino acid or nucleic acid sequences of the CRISPR nucleases.

In an embodiment, the one or more NLSs are in tandem repeats.

In an embodiment, the one or more NLSs are considered in proximity to the N- or C-terminus when the nearest amino acid of the NLS is within about 1, 2, 3, 4, 5, 10, 15, 20, 25, 30, 40, 50, or more amino acids along the polypeptide chain from the N- or C-terminus.

As discussed, the CRISPR nuclease may be engineered to comprise one or more of a nuclear localization sequences (NLS), cell penetrating peptide sequences, and/or affinity tags.

In an embodiment, the CRISPR nuclease exhibits increased specificity to a target site compared to the wild-type of the CRISPR nuclease when complexed with the one or more RNA molecules.

In an embodiment, the complex of the CRISPR nuclease and one or more RNA molecules exhibits at least maintained on-target editing activity of the target site and reduced off-target activity compared to the wild-type of the CRISPR nuclease.

In an embodiment, the composition further comprises a recombinant nucleic acid molecule comprising a heterologous promoter operably linked to the nucleotide acid molecule comprising the sequence encoding the CRISPR nuclease.

In an embodiment, the CRISPR nuclease or nucleic acid molecule comprising a sequence encoding the CRISPR nuclease is non-naturally occurring or engineered.

This invention also provides a non-naturally occurring or engineered composition comprising a vector system comprising the nucleic acid molecule comprising a sequence encoding any of the CRISPR nucleases of the invention.

This invention also provides use of any of the compositions of the invention for the treatment of a subject afflicted with a disease associated with a genomic mutation comprising modifying a nucleotide sequence at a target site in the genome of the subject.

This invention provides a method of modifying a nucleotide sequence at a target site in the genome of a mammalian cell comprising introducing into the cell (i) a composition comprising a CRISPR nuclease having at least 95% identity to the amino acid sequence of SEQ ID NO: 3 or a nucleic acid molecule comprising a sequence encoding a CRISPR nuclease which sequence has at least 95% identity to a nucleic acid sequence selected from the group consisting of SEQ ID NOs: 12 or 13 and (ii) a DNA-targeting RNA molecule, or a DNA polynucleotide encoding a DNA-targeting RNA molecule, comprising a nucleotide sequence that is complementary to a sequence in the target DNA.

In some embodiments, the method is performed ex vivo. In some embodiments, the method is performed in vivo. In some embodiments, some steps of the method are performed ex vivo and some steps are performed in vivo. In some embodiments the mammalian cell is a human cell.

In an embodiment, the method further comprises introducing into the cell: (iii) an RNA molecule comprising a nuclease-binding RNA sequence or a DNA polynucleotide encoding an RNA molecule comprising a nuclease-binding RNA that interacts with the CRISPR nuclease.

In an embodiment, the DNA targeting RNA molecule is a crRNA molecule suitable to form an active complex with the CRISPR nuclease.

In an embodiment, the RNA molecule comprising a nuclease-binding RNA sequence is a tracrRNA molecule suitable to form an active complex with the CRISPR nuclease.

In an embodiment, the DNA-targeting RNA molecule and the RNA molecule comprising a nuclease-biding RNA sequence are fused in the form of a single guide RNA molecule.

In an embodiment, the method further comprises introducing into the cell: (iv) an RNA molecule comprising a sequence complementary to a protospacer sequence.

In an embodiment, the CRISPR nuclease forms a complex with the one or more RNA molecules and effects a double strand break in the 3′ of a Protospacer Adjacent Motif (PAM).

In an embodiment, the CRISPR nuclease forms a complex with the one or more RNA molecules and effects a double strand break in the 5′ of a Protospacer Adjacent Motif (PAM).

In some embodiments, (a) the CRISPR nuclease has at least 100%, 99%, 98%, 97%, 96%, 95%, 94%, 93%, 92%, 91%, 90%, 85%, 80% identity to SEQ ID NO: 3, or (b) the nucleic acid molecule comprising a sequence encoding the CRISPR nuclease comprises a sequence of at least a 95% sequence identity to the nucleic acid sequence as set forth in SEQ ID NO: 11, 12, or 13, and the PAM is NGG. Non-limiting examples of suitable PAM sequences include: GGG, AGG, and TGG. In this embodiment, the nucleotide sequence that can form a complex with the CRISPR nuclease in the DNA-targeting RNA molecule comprises a sequence selected from SEQ ID NOs: 37-45, 87-88, 149-154 and GUUUGAGAG.

In some embodiments, the CRISPR nuclease utilizes a PAM having a sequence of NAG or NGA.

In an embodiment of any of the methods described herein, the method is for treating a subject afflicted with a disease associated with a genomic mutation comprising modifying a nucleotide sequence at a target site in the genome of the subject.

In an embodiment, the method comprises first selecting a subject afflicted with a disease associated with a genomic mutation and obtaining the cell from the subject.

This invention also provides a modified cell or cells obtained by any of the methods described herein. In an embodiment these modified cell or cells are capable of giving rise to progeny cells. In an embodiment these modified cell or cells are capable of giving rise to progeny cells after engraftment.

This invention also provides a composition comprising these modified cells and a pharmaceutically acceptable carrier. Also provided is an in vitro or ex vivo method of preparing this, comprising mixing the cells with the pharmaceutically acceptable carrier.

### DNA-Targeting RNA Molecules

In embodiments of the present invention, the DNA-targeting RNA sequence comprises a guide sequence portion. The “guide sequence portion” of an RNA molecule refers to a nucleotide sequence that is capable of hybridizing to a specific target DNA sequence, e.g., the guide sequence portion has a nucleotide sequence which is fully complementary to the DNA sequence being targeted along the length of the guide sequence portion. In some embodiments, the guide sequence portion is 17, 18, 19, 20, 21, 22, 23, or 24 nucleotides in length, or approximately 17-24, 18-22, 19-22, 18-20, 17-20, or 21-22 nucleotides in length. The entire length of the guide sequence portion is fully complementary to the DNA sequence being targeted along the length of the guide sequence portion. The guide sequence portion may be part of an RNA molecule that can form a complex with a CRISPR nuclease with the guide sequence portion serving as the DNA targeting portion of the CRISPR complex. When the RNA molecule having the guide sequence portion is present contemporaneously with the CRISPR molecule, the RNA molecule is capable of targeting the CRISPR nuclease to the specific target DNA sequence. Each possibility represents a separate embodiment. An RNA molecule can be custom designed to target any desired sequence.

In embodiments of the present invention, the CRISPR nuclease has greater cleavage activity when used with an RNA molecule comprising a guide sequence portion having 21-23 nucleotides, compared to its cleavage activity when used with an RNA molecule comprising a guide sequence portion having 20 or fewer nucleotides, and/or 24 or more nucleotides. In embodiments of the present invention, the CRISPR nuclease has greater cleavage activity when used with an RNA molecule comprising a guide sequence portion having 21-22 nucleotides, compared to its cleavage activity when used with an RNA molecule comprising a guide sequence portion having 20 or fewer nucleotides, and/or 23 or more nucleotides. In an embodiment, the CRISPR nuclease has its greatest cleavage activity when used with an RNA molecule comprising a guide sequence portion having 22 nucleotides.

In an embodiment, such a CRISPR nuclease has at least 95% identity to the amino acid sequence as set forth in SEQ ID NO: 3 or the sequence encoding the CRISPR nuclease has at least a 95% sequence identity to any of SEQ ID NOs: 11-13. In an embodiment, such a CRISPR nuclease has at least 100%, 99.5%, 99%, 98%, 97%, 96%, 95%, 94%, 93%, 92%, 91%, 90%, 89%, 88%, 87%, 86%, 85%, 84%, 83%, or 82% identity to the amino acid sequence as set forth in SEQ ID NO: 3 or the sequence encoding the CRISPR nuclease has at least a 100%, 99%, 98%, 97%, 96%, 95%, 94%, 93%, 92%, 91%, 90%, 89%, 88%, 87%, 86%, 85%, 84%, 83%, or 82% sequence identity to any of SEQ ID NOs: 11-13.

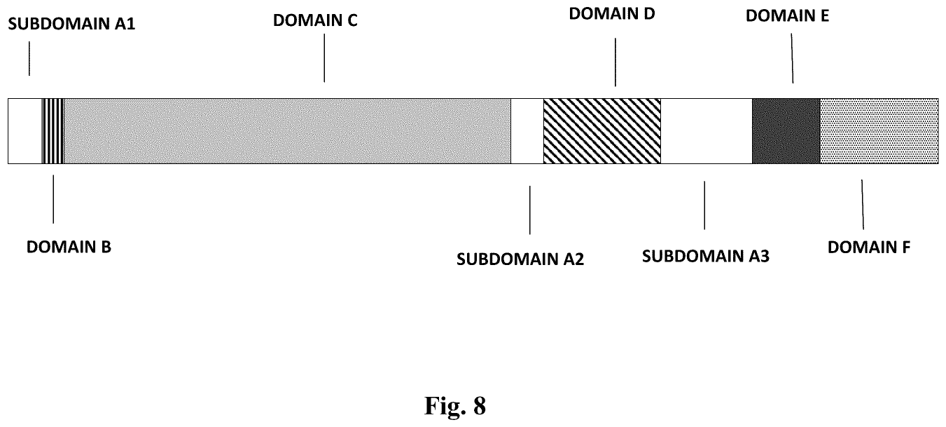

The characteristic targeted nuclease activity of a CRISPR nuclease is imparted by the various functions of its specific domains. In this application the OMNI-50 domains are defined as Domain A, Domain B, Domain C, Domain D, Domain E, and Domain F as presented in the FIG. 8 OMNI-50 schematic.

As used herein, Domain A comprises three subdomains: Subdomain A1, Subdomain A2, and Subdomain A3. As used herein, Subdomain A1 begins at an amino acid position within 1-10 and ends at an amino acid position within 45-55 of SEQ ID NO: 3; Subdomain A2 begins at an amino acid position within 736-746 and ends at an amino acid position within 784-794 of SEQ ID NO: 3; and Subdomain A3 begins at an amino acid position within 957-967 and ends at an amino acid position within 1091-1101 of SEQ ID NO: 3. Based on a preferred analysis of a local alignment generated using the Smith-Waterman algorithm, in an embodiment Subdomain A1 has been identified as amino acids 1 to 50 of SEQ ID NO: 3, Subdomain A2 has been identified as amino acids 741 to 789 of SEQ ID NO: 3, and Subdomain A3 has been identified as amino acids 962 to 1096 of SEQ ID NO: 3.

As used herein, Domain B begins at an amino acid position within 46-56 and ends at an amino acid position within 78-88 of SEQ ID NO: 3. Based on a preferred analysis of a local alignment generated using the Smith-Waterman algorithm, in an embodiment Domain B has been identified as amino acids 51 to 83 of SEQ ID NO: 3.

As used herein, Domain C comprises three subdomains: Subdomain C1, Subdomain C2, and Subdomain C3, or alternatively two subdomains: Subdomain Ca and Subdomain Cb. As used herein, Subdomain C1 begins at an amino acid position within 79-89 and ends at an amino acid position within 155-165 of SEQ ID NO: 3; Subdomain C2 begins at an amino acid position within 156-166 and ends at an amino acid position within 294-304 of SEQ ID NO: 3; and Subdomain C3 begins at an amino acid position within 295-305 and ends at an amino acid position within 732-742 of SEQ ID NO: 3. Based on a preferred analysis of a local alignment generated using the Smith-Waterman algorithm, in an embodiment Subdomain C1 has been identified as amino acids 84-160 of SEQ ID NO: 3, Subdomain C2 has been identified as amino acids 161-299 of SEQ ID NO: 3, and Subdomain C3 has been identified as amino acids 300-737 of SEQ ID NO: 3. As used herein, Subdomain Ca begins at an amino acid position within 79-89 and ends at an amino acid position within 473-483 of SEQ ID NO: 3; and Subdomain Cb begins at an amino acid position within 474-484 and ends at an amino acid position within 732-742 of SEQ ID NO: 3. Based on an analysis of a local alignment generated using the Smith-Waterman algorithm, in an embodiment Subdomain Ca has been identified as amino acids 84-478 of SEQ ID NO: 3 and Subdomain Cb has been identified as amino acids 479-737 of SEQ ID NO: 3.

As used herein, Domain D begins at an amino acid position within 785-795 and ends at an amino acid position within 956-966 of SEQ ID NO: 3. Based on a preferred analysis of a local alignment generated using the Smith-Waterman algorithm, in an embodiment Domain D has been identified as amino acids 790 to 961 of SEQ ID NO: 3.

As used herein, Domain E begins at an amino acid position within 1092-1102 and ends at an amino acid position within 1191-1201 of SEQ ID NO: 3. Based on a preferred analysis of a local alignment generated using the Smith-Waterman algorithm, in an embodiment Domain E has been identified as amino acids 1097 to 1196 of SEQ ID NO: 3.

As used herein, Domain F begins at an amino acid position within 1192-1202 and ends at an amino acid position within 1360-1370 of SEQ ID NO: 3. Based on a preferred analysis of a local alignment generated using the Smith-Waterman algorithm, in an embodiment Domain F has been identified as amino acids 1197 to 1370 of SEQ ID NO: 3.

The activity of each OMNI-50 nuclease domain is described herein, with each domain activity providing aspects of the advantageous features of the nuclease.

Specifically, OMNI-50 Domain A and contains a nuclease active site that participates in DNA strand cleavage. Domain A cleaves a DNA strand that a targeting RNA molecule binds at a DNA target site.

Domain B is involved in initiating DNA cleavage activity upon OMNI-50 binding to a target a DNA site.

Domain C binds a targeting RNA molecule and participates in providing specificity for target site recognition. Domain C comprises Subdomain C1, Subdomain C2, and Subdomain C3, which each participate in specific functional aspects of Domain C activity. For example, C3 is involved in sensing a DNA target site; C2 is involved in regulating the activation of a nuclease domain (e.g. Domain D); and C1 is involved in locking the nuclease domain at the target site. Accordingly, Domain C participates in controlling cleavage of off-target sequences.

Domain D contains a nuclease active site that participates in DNA strand cleavage. Domain D cleaves a DNA strand that is displaced by a targeting RNA molecule binding at a DNA target site.

Domain E is structurally similar to a topoisomerase domain.

Domain F is involved in providing PAM site specificity, including aspects of PAM site interrogation and recognition.

Further description of other CRISPR nuclease domains and their general functions can be found in, inter alia, Mir et al., ACS Chem. Biol. (2019), Palermo et al., Quarterly Reviews of Biophysics (2018), Jiang and Doudna, Annual Review of Biophysics (2017), Nishimasu et al., Cell (2014) and Nishimasu et al., Cell (2015), incorporated herein by reference.

In one aspect of the invention, an amino acid sequence having similarity to an OMNI-50 domain or subdomain may be utilized in the design and manufacture of a non-naturally occurring peptide, e.g. a CRISPR nuclease, such that the peptide displays the advantageous feature of the OMNI-50 domain or subdomain activity.

In an embodiment, such a peptide, e.g. a CRISPR nuclease, comprises an amino acid sequence that has at least 100%, 99.5%, 99%, 98%, 97%, 96%, 95%, 94%, 93%, 92%, 91%, 90%, 89%, 88%, 87%, 86%, 85%, 84%, 83%, or 82% identity to the amino acid sequence of at least one of Domain A or any one of its three subdomains, Domain B, Domain C or any one of its three subdomains, Domain D, Domain E, or Domain F of the OMNI-50 nuclease. In an embodiment, the peptide exhibits extensive amino acid variability relative to the full length OMNI-50 amino acid sequence (SEQ ID NO: 3) outside of the peptide amino acid sequence having at least 100%, 99.5%, 99%, 98%, 97%, 96%, 95%, 94%, 93%, 92%, 91%, 90%, 89%, 88%, 87%, 86%, 85%, 84%, 83%, or 82% identity to the amino acid sequence of at least one of Domain A or any one of its three subdomains, Domain B, Domain C or any one of its three subdomains, Domain D, Domain E, or Domain F of the OMNI-50 nuclease. In an embodiment, the peptide comprises an intervening amino acid sequence between two domain sequences. In an embodiment, the intervening amino acid sequence is 1-10, 10-20, 20-40, 40-50 or up to 100 amino acids in length. In an embodiment, the intervening sequence is a linker sequence.

In one aspect of the invention, an amino acid sequence encoding any one of the domains of the OMNI-50 nuclease described herein in the peptide may comprise one or more amino acid substitutions relative to the original OMNI-50 domain sequence. The amino acid substitution may be a conservative substitution, i.e. substitution for an amino acid having similar chemical properties as the original amino acid. For example, a positively charged amino acid may be substituted for an alternate positively charged amino acid, e.g. an arginine residue may be substituted for a lysine residue, or a polar amino acid may be substituted for a different polar amino acid. Conservative substitutions are more tolerable, and the amino acid sequence encoding any one of the domains of the OMNI-50 nuclease may contain as many as 10% of such substitutions. The amino acid substitution may be a radical substitution, i.e. substitution for an amino acid having different chemical properties as the original amino acid. For example, a positively charged amino acid may be substituted for a negatively charged amino acid, e.g. an arginine residue may be substituted for a glutamic acid residue, or a polar amino acid may be substituted for a non-polar amino acid. The amino acid substitution may be a semi-conservative substitution, or the amino acid substitution may be to any other amino acid. The substitution may alter the activity relative to the original OMNI-50 domain function e.g. reduce catalytic nuclease activity.

According to some aspects of the invention, the disclosed compositions comprise a non-naturally occurring composition comprising a CRISPR nuclease, wherein the CRISPR nuclease comprises an amino acid sequence corresponding to the amino acid sequence of at least one of Domain A, Domain B, Domain C, Domain D, Domain E, or Domain F of the OMNI-50 nuclease. In some embodiments of the invention, the CRISPR nuclease comprises at least one, at least two, at least three, at least four, or at least five amino acid sequences, wherein each amnio acid sequence corresponds to any one of the amino acid sequences of Domain A, Domain B, Domain C, Domain D, Domain E, or Domain F of the OMNI-50 nuclease. Accordingly, the CRISPR nuclease may include any combination of amino acid sequences that corresponding to any of Domain A, Domain B, Domain C, Domain D, Domain E, or Domain F of the OMNI-50 nuclease.

In some embodiments, the CRISPR nuclease comprises a Domain A which comprises at least one of
a) Subdomain A1 having at least 97% sequence identity to amino acids 1 to 50 of SEQ ID NO: 3;
b) Subdomain A2 having at least 97% sequence identity to amino acids 741 to 789 of SEQ ID NO: 3; or
c) Subdomain A3 having at least 97% sequence identity to amino acids 962 to 1096 of SEQ ID NO: 3.

In some embodiments, the CRISPR nuclease comprises a Domain B having at least 97% sequence identity to amino acids 51 to 83 of SEQ ID NO: 3.

In some embodiments, the CRISPR nuclease comprises a Domain C which comprises at least one of
a) Subdomain C1 having at least 97% sequence identity to amino acids 84 to 160 of SEQ ID NO: 3;
b) Subdomain C2 having at least 97% sequence identity to amino acids 161 to 299 of SEQ ID NO: 3; or
c) Subdomain C3 having at least 97% sequence identity to amino acids 300 to 737 of SEQ ID NO: 3.

In some embodiments, the CRISPR nuclease comprises a Domain C which comprises at least one of
a) Subdomain Ca having at least 97% sequence identity to amino acids 84 to 478 of SEQ ID NO: 3; and
b) Subdomain Cb having at least 97% sequence identity to amino acids 479 to 737 of SEQ ID NO: 3.

In some embodiments, Domain C has at least 97% sequence identity to amino acids 84 to 737 of SEQ ID NO: 3.

In some embodiments, the CRISPR nuclease comprises a Domain D having at least 97% sequence identity to amino acids 790 to 961 of SEQ ID NO: 3.

In some embodiments, the CRISPR nuclease comprises a Domain E having at least 97% sequence identity to amino acids 1097 to 1196 of SEQ ID NO: 3.

In some embodiments, the CRISPR nuclease comprises a Domain F having at least 97% sequence identity to amino acids 1197 to 1370 of SEQ ID NO: 3.

In some embodiments, the CRISPR nuclease comprises Domain A, Domain B, Domain C, Domain D, Domain E, and Domain F, wherein
a) Domain A comprises
   i. Subdomain A1 having at least 97% sequence identity to amino acids 1 to 50 of SEQ ID NO: 3;
   ii. Subdomain A2 having at least 97% sequence identity to amino acids 741 to 789 of SEQ ID NO: 3; and
   iii. Subdomain A3 having at least 97% sequence identity to amino acids 962 to 1096 of SEQ ID NO: 3;
b) Domain B has at least 97% sequence identity to amino acids 51 to 83 of SEQ ID NO: 3;
c) Domain C has at least 97% sequence identity to amino acids 84 to 737 of SEQ ID NO: 3;
d) Domain D has at least 97% sequence identity to amino acids 790 to 961 of SEQ ID NO: 3;
e) Domain E has at least 97% sequence identity to amino acids 1097 to 1196 of SEQ ID NO: 3; and
f) Domain F has at least 97% sequence identity to amino acids 1197 to 1370 of SEQ ID NO: 3.

In some embodiments, the CRISPR nuclease sequence is at least 100-250, 250-500, 500-1000, or 1000-2000 amino acids in length.

According to some aspects of the invention, the disclosed compositions comprise a non-naturally occurring composition comprising a peptide, wherein the peptide comprises an amino acid sequence having at least 97% sequence identity to the amino acid sequence of at least one of Domain A, Domain B, Domain C, Domain D, Domain E, or Domain F of the OMNI-50 nuclease.

In some embodiments, the amino acid sequence of Domain A comprises an amino acid sequence of at least one of
a) Subdomain A1 having at least 97% sequence identity to amino acids 1 to 50 of SEQ ID NO: 3;
b) Subdomain A2 having at least 97% sequence identity to amino acids 741 to 789 of SEQ ID NO: 3; or
c) Subdomain A3 having at least 97% sequence identity to amino acids 962 to 1096 of SEQ ID NO: 3.

In some embodiments, the amino acid sequence of Domain B has at least 97% sequence identity to amino acids 51 to 83 of SEQ ID NO: 3.

In some embodiments, the amino acid sequence of Domain C comprises an amino acid sequence of at least one of
a) Subdomain C1 having at least 97% sequence identity to amino acids 84 to 160 of SEQ ID NO: 3;
b) Subdomain C2 having at least 97% sequence identity to amino acids 161 to 299 of SEQ ID NO: 3; or
c) Subdomain C3 having at least 97% sequence identity to amino acids 300 to 737 of SEQ ID NO: 3.

In some embodiments, the amino acid sequence of Domain C comprises an amino acid sequence of at least one of
a) Subdomain Ca having at least 97% sequence identity to amino acids 84 to 478 of SEQ ID NO: 3; and
b) Subdomain Cb having at least 97% sequence identity to amino acids 479 to 737 of SEQ ID NO: 3.

In some embodiments, the amino acid sequence of Domain C has at least 97% sequence identity to amino acids 84 to 737 of SEQ ID NO: 3.

In some embodiments, the amino acid sequence of Domain D has at least 97% sequence identity to amino acids 790 to 961 of SEQ ID NO: 3.

In some embodiments, the amino acid sequence of Domain E has at least 97% sequence identity to amino acids 1097 to 1196 of SEQ ID NO: 3.

In some embodiments, the amino acid sequence of Domain F has at least 97% sequence identity to amino acids 1197 to 1370 of SEQ ID NO: 3.

In some embodiments, the amino acid sequence is at least 100-250, 250-500, 500-1000, or 1000-2000 amino acids in length.

According to some aspects of the invention, the disclosed compositions comprise a non-naturally occurring composition comprising a polynucleotide encoding an amino acid sequence having at least 97% sequence identity to the amino acid sequence of at least one of Domain A, Domain B, Domain C, Domain D, Domain E, or Domain F of the OMNI-50 nuclease.

According to some aspects of the invention, the disclosed compositions comprise a non-naturally occurring amino acid sequence having at least 97% sequence identity to the amino acid sequence of at least one of Domain A, Domain B, Domain C, Domain D, Domain E, or Domain F of the OMNI-50 nuclease.

According to some aspects of the invention, the disclosed methods comprise a method of modifying a nucleotide sequence at a target site in a cell-free system or the genome of a cell comprising introducing into the cell the composition of any one of the embodiments described herein.

In some embodiments, the cell is a eukaryotic cell, preferably a mammalian cell or a plant cell.

According to some aspects of the invention, the disclosed methods comprise a use of any one of the compositions described herein for the treatment of a subject afflicted with a disease associated with a genomic mutation comprising modifying a nucleotide sequence at a target site in the genome of the subject.

According to some aspects of the invention, the disclosed methods comprise a method of treating subject having a mutation disorder comprising targeting any one of the compositions described herein to an allele associated with the mutation disorder.

In some embodiments, the mutation disorder is related to a disease or disorder selected from any of a neoplasia, age-related macular degeneration, schizophrenia, neurological, neurodegenerative, or movement disorder, Fragile X Syndrome, secretase-related disorders, prion-related disorders, ALS, addiction, autism, Alzheimer's Disease, neutropenia, inflammation-related disorders, Parkinson's Disease, blood and coagulation diseases and disorders, cell dysregulation and oncology diseases and disorders, inflammation and immune-related diseases and disorders, metabolic, liver, kidney and protein diseases and disorders, muscular and skeletal diseases and disorders, dermatological diseases and disorders, neurological and neuronal diseases and disorders, and ocular diseases and disorders.

In some embodiments, the mutation disorder is beta thalassemia or sickle cell anemia.

In some embodiments, the allele associated with the disease is BCL11A.

### Diseases and Therapies

Certain embodiments of the invention target a nuclease to a specific genetic locus associated with a disease or disorder as a form of gene editing, method of treatment, or therapy. For example, to induce editing or knockout of a gene, a novel nucleases disclosed herein may be specifically targeted to a pathogenic mutant allele of the gene using a custom designed guide RNA molecule. The guide RNA molecule is preferably designed by first considering the PAM requirement of the nuclease, which as shown herein is also dependent on the system in which the gene editing is being performed. For example, a guide RNA molecule designed to target an OMNI-50 nuclease to a target site is designed to contain a spacer region complementary to a region neighboring the OMNI-50 PAM sequence “NGG.” The guide RNA molecule is further preferably designed to contain a spacer region (i.e. the region of the guide RNA molecule having complementarity to the target allele) of sufficient and preferably optimal length in order to increase specific activity of the nuclease and reduce off-target effects. For example, a guide RNA molecule designed to target OMNI-50 nuclease may be designed to contain a 22 nt spacer for high on-target cleavage activity.

As a non-limiting example, the guide RNA molecule may be designed to target the nuclease to a specific region of a mutant allele, e.g. near the start codon, such that upon DNA damage caused by the nuclease a non-homologous end joining (NHEJ) pathway is induced and leads to silencing of the mutant allele by introduction of frameshift mutations. This approach to guide RNA molecule design is particularly useful for altering the effects of dominant negative mutations and thereby treating a subject. As a separate non-limiting example, the guide RNA molecule may be designed to target a specific pathogenic mutation of a mutated allele, such that upon DNA damage caused by the nuclease a homology directed repair (HDR) pathway is induced and leads to template mediated correction of the mutant allele. This approach to guide RNA molecule design is particularly useful for altering haploinsufficiency effects of a mutated allele and thereby treating a subject.

Non-limiting examples of specific genes which may be targeted for alteration to treat a disease or disorder are presented herein below. Specific disease-associated genes and mutations that induce a mutation disorder are described in the literature. Such mutations can be used to design a DNA-targeting RNA molecule to target a CRISPR composition to an allele of the disease associated gene, where the CRISPR composition causes DNA damage and induces a DNA repair pathway to alter the allele and thereby treat the mutation disorder.

Mutations in the ELANE gene are associated with neutropenia. Accordingly, without limitation, embodiments of the invention that target ELANE may be used in methods of treating subjects afflicted with neutropenia.

CXCR4 is a co-receptor for the human immunodeficiency virus type 1 (HIV-1) infection. Accordingly, without limitation, embodiments of the invention that target CXCR4 may be used in methods of treating subjects afflicted with HIV-1 or conferring resistance to HIV-1 infection in a subject.

Programmed cell death protein 1 (PD-1) disruption enhances CAR-T cell mediated killing of tumor cells and PD-1 may be a target in other cancer therapies. Accordingly, without limitation, embodiments of the invention that target PD-1 may be used in methods of treating subjects afflicted with cancer. In an embodiment, the treatment is CAR-T cell therapy with T cells that have been modified according to the invention to be PD-1 deficient.

In addition, BCL11A is a gene that plays a role in the suppression of hemoglobin production. Globin production may be increased to treat diseases such as thalassemia or sickle cell anemia by inhibiting BCL11A. See for example, PCT International Publication No. WO 2017/077394A2; U.S. Publication No. US2011/0182867A1; Humbert et al. Sci. Transl. Med. (2019); and Canver et al. Nature (2015). Accordingly, without limitation, embodiments of the invention that target an enhancer of BCL11A may be used in methods of treating subjects afflicted with beta thalassemia or sickle cell anemia.

Embodiments of the invention may also be used for targeting any disease-associated gene, for studying, altering, or treating any of the diseases or disorders listed in Table A or Table B below. Indeed, any disease-associated with a genetic locus may be studied, altered, or treated by using the nucleases disclosed herein to target the appropriate disease-associated gene, for example, those listed in U.S. Publication No. 2018/0282762A1 and European Patent No. EP3079726B1.

TABLE A
Diseases, Disorders and their associated genes

| DISEASE/DISORDERS | GENE(S) |
|---|---|
| Neoplasia | PTEN; ATM; ATR; EGFR; ERBB2; ERBB3; ERBB4; Notch1; |
| | Notch2; Notch3; Notch4; AKT; AKT2; AKT3; HIF; HIF1a; |
| | HIF3a; Met; HRG; Bcl2; PPAR alpha; PPAR gamma; WT1 |
| | (Wilms Tumor); FGF Receptor Family members (5 members: 1, |
| | 2, 3, 4, 5); CDKN2a; APC; RB (retinoblastoma); MEN1; VHL; |
| | BRCA1; BRCA2; AR (Androgen Receptor); TSG101; IGF; IGF |
| | Receptor; Igf1 (4 variants); gf2 (3 variants); Igf 1 Receptor; Igf 2 |
| | Receptor; Bax; Bcl2; caspases family (9 members: 1, 2, 3, 4, 6, |
| | 7, 8, 9, 12); Kras; Apc |
| Age-related Macular | Abcr; Ccl2; Cc2; cp (ceruloplasmin); Timp3; cathepsinD; Vldlr; |
| Degeneration | Ccr2 |
| Schizophrenia | Neuregulin1 (Nrg1); Erb4 (receptor for Neuregulin); |
| | Complexin1 (Cp1x1); Tph1 Tryptophan hydroxylase; Tph2 |
| | Tryptophan hydroxylase 2; Neurexin 1; GSK3; GSK3a; GSK3b |
| Neurological, Neuro | 5-HTT (S1c6a4); COMT; DRD (Drd1a); SLC6A3; DAOA; |
| degenerative, and | DTNBP1; Dao (Dao1) |
| Movement Disorders | |
| Trinucleotide Repeat | HTT (Huntington's Dx); SBMA/SMAX1/AR (Kennedy's Dx); |
| Disorders | FXN/X25 (Friedrich's Ataxia); ATX3 (Machado-Joseph's Dx); |
| | ATXN1 and ATXN2 (spinocerebellar ataxias); DMPK |
| | (myotonic dystrophy); Atrophin-1 and Atn1 (DRPLA Dx); CBP |
| | (Creb-BP-global instability); VLDLR (Alzheimer's); Atxn7; |
| | Atxn10 |
| Fragile X Syndrome | FMR2; FXR1; FXR2; mGLUR5 |
| Secretase Related | APH-1 (alpha and beta); Presenilin (Psen1); nicastrin (Ncstn); |
| Disorders | PEN-2 |
| Others | Nos1; Parp1; Nat1; Nat2 |
| Prion related disorders | Prp |
| ALS | SOD1; ALS2; STEX; FUS; TARDBP; VEGF (VEGF-a; VEGF- |
| | b; VEGF-c) |
| Addiction | Prkce (alcohol); Drd2; Drd4; ABAT (alcohol); GRIA2; Grm5; |
| | Grin1; Htr1b; Grin2a; Drd3; Pdyn; Gria1 (alcohol) |
| Autism | Mecp2; BZRAP1; MDGA2; Sema5A; Neurexin 1; Fragile X |
| | (FMR2 (AFF2); FXR1; FXR2; Mglur5) |
| Alzheimer's Disease | E1; CHIP; UCH; UBB; Tau; LRP; PICALM; Clusterin; PS1; |
| | SORL1; CR1; Vldlr; Uba1; Uba3; CHIP28 (Aqp1, Aquaporin |
| | 1); Uchl1; Uchl3; APP |
| Inflammation | IL-10; IL-1 (IL-1a; IL-1b); IL-13; IL-17 (IL-17a (CTLA8); IL- |
| | 17b; IL-17c; IL-17d; IL-17f); II-23; Cx3cr1; ptpn22; TNFa; |
| | NOD2/CARD15 for IBD; IL-6; IL-12 (IL-12a; IL-12b); CTLA4; |
| | Cx3cl1 |
| Parkinson's Disease | x-Synuclein; DJ-1; LRRK2; Parkin; PINK1 |

TABLE B
Diseases, Disorders and their associated genes

| DISEASE CATEGORY | DISEASE AND ASSOCIATED GENES |
|---|---|
| Blood and coagulation | Anemia (CDAN1, CDA1, RPS19, DBA, PKLR, PK1, NT5C3, |
| diseases and disorders | UMPH1, PSN1, RHAG, RH50A, NRAMP2, SPTB, ALAS2, |
| | ANH1, ASB, ABCB7, ABC7, ASAT); Bare lymphocyte |
| | syndrome (TAPBP, TPSN, TAP2, ABCB3, PSF2, RING11, |
| | MHC2TA, C2TA, RFX5, RFXAP, RFX5), Bleeding disorders |
| | (TBXA2R, P2RX1, P2X1); Factor H and factor H-like 1 (HF1, |
| | CFH, HUS); Factor V and factor VIII (MCFD2); Factor VII |
| | deficiency (F7); Factor X deficiency (F10); Factor XI deficiency |
| | (F11); Factor XII deficiency (F12, HAF); Factor XIIIA |
| | deficiency (F13A1, F13A); Factor XIIIB deficiency (F13B); |
| | Fanconi anemia (FANCA, FACA, FA1, FA, FAA, FAAP95, |
| | FAAP90, FLJ34064, FANCB, FANCC, FACC, BRCA2, |
| | FANCD1, FANCD2, FANCD, FACD, FAD, FANCE, FACE, |
| | FANCF, XRCC9, FANCG, BRIP1, BACH1, FANCJ, PHF9, |
| | FANCL, FANCM, KIAA1596); Hemophagocytic |
| | lymphohistiocytosis disorders (PRF1, HPLH2, UNC13D, |
| | MUNC13-4, HPLH3, HLH3, FHL3); Hemophilia A (F8, F8C, |
| | HEMA); Hemophilia B (F9, HEMB), Hemorrhagic disorders |
| | (PI, ATT, F5); Leukocyde deficiencies and disorders (ITGB2, |
| | CD18, LCAMB, LAD, EIF2B1, EIF2BA, EIF2B2, EIF2B3, |
| | EIF2B5, LVWM, CACH, CLE, EIF2B4); Sickle cell anemia |
| | (HBB); Thalassemia (HBA2, HBB, HBD, LCRB, HBA1) |
| Cell dysregulation and | B-cell non-Hodgkin lymphoma (BCL7A, BCL7); Leukemia |
| oncology diseases and | (TAL1, TCL5, SCL, TAL2, FLT3, NBS1, NBS, ZNFN1A1, |
| disorders | IK1, LYF1, HOXD4, HOX4B, BCR, CML, PHL, ALL, ARNT, |
| | KRAS2, RASK2, GMPS, AF10, ARHGEF12, LARG, |
| | KIAA0382, CALM, CLTH, CEBPA, CEBP, CHIC2, BTL, |
| | FLT3, KIT, PBT, LPP, NPM1, NUP214, D9546E, CAN, CAIN, |
| | RUNX1, CBFA2, AML1, WHSC1L1, NSD3, FLT3, AF1Q, |
| | NPM1, NUMA1, ZNF145, PLZF, PML, MYL, STAT5B, AF10, |
| | CALM, CLTH, ARL11, ARLTS1, P2RX7, P2X7, BCR, CML, |
| | PHL, ALL, GRAF, NF1, VRNF, WSS, NFNS, PTPN11, |
| | PTP2C, SHP2, NS1, BCL2, CCND1, PRAD1, BCL1, TCRA, |
| | GATA1, GF1, ERYF1, NFE1, ABL1, NQO1, DIA4, NMOR1, |
| | NUP214, D9546E, CAN, CAIN) |
| Inflammation and immune | AIDS (KIR3DL1, NKAT3, NKB1, AMB11, KIR3DS1, IFNG, |
| related diseases and | CXCL12, SDF1); Autoimmune lymphoproliferative syndrome |
| disorders | (TNFRSF6, APT1, FAS, CD95, ALPS1A); Combined |
| | immunodeficiency, (IL2RG, SCIDX1, SCIDX, IMD4); HIV-1 |
| | (CCL5, SCYA5, D175136E, TCP228), HIV susceptibility or |
| | infection (IL10, CSIF, CMKBR2, CCR2, CMKBR5, CCCKR5 |
| | (CCR5)); Immunodeficiencies (CD3E, CD3G, AICDA, AID, |
| | HIGM2, TNFRSF5, CD40, UNG, DGU, HIGM4, TNFSF5, |
| | CD40LG, HIGM1, IGM, FOXP3, IPEX, AIID, XPID, PIDX, |
| | TNFRSF14B, TACI); Inflammation (IL-10, IL-1 (IL-1a, IL-1b), |
| | IL-13, IL-17 (IL-17a (CTLA8), IL-17b, IL-17c, IL-17d, IL-17f), |
| | II-23, Cx3cr1, ptpn22, TNFa, NOD2/CARD15 for IBD, |
| | IL-6, IL-12 (IL-12a, IL-12b), CTLA4, Cx3cl1); Severe |
| | combined immunodeficiencies (SCIDs)(JAK3, JAKL, |
| | DCLRE1C, ARTEMIS, SCIDA, RAG1, RAG2, ADA, PTPRC, |
| | CD45, LCA, IL7R, CD3D, T3D, IL2RG, SCIDX1, SCIDX, |
| | IMD4) |
| Metabolic, liver, kidney | Amyloid neuropathy (TTR, PALB); Amyloidosis (APOA1, |
| and protein diseases and | APP, AAA, CVAP, AD1, GSN, FGA, LYZ, TTR, PALB); |
| disorders | Cirrhosis (KRT18, KRT8, CIRH1A, NAIC, TEX292, |
| | KIAA1988); Cystic fibrosis (CFTR, ABCC7, CF, MRP7); |
| | Glycogen storage diseases (SLC2A2, GLUT2, G6PC, G6PT, |
| | G6PT1, GAA, LAMP2, LAMPB, AGL, GDE, GBE1, GYS2, |
| | PYGL, PFKM); Hepatic adenoma, 142330 (TCF1, HNF1A, |
| | MODY3), Hepatic failure, early onset, and neurologic disorder |
| | (SCOD1, SCO1), Hepatic lipase deficiency (LIPC), |
| | Hepatoblastoma, cancer and carcinomas (CTNNB1, PDGFRL, |
| | PDGRL, PRLTS, AXIN1, AXIN, CTNNB1, TP53, P53, LFS1, |
| | IGF2R, MPRI, MET, CASP8, MCH5; Medullary cystic kidney |
| | disease (UMOD, HNFJ, FJHN, MCKD2, ADMCKD2); |
| | Phenylketonuria (PAH, PKU1, QDPR, DHPR, PTS); Polycystic |
| | kidney and hepatic disease (FCYT, PKHD1, ARPKD, PKD1, |
| | PKD2, PKD4, PKDTS, PRKCSH, G19P1, PCLD, SEC63) |
| Muscular/Skeletal | Becker muscular dystrophy (DMD, BMD, MYF6), Duchenne |
| diseases and disorders | Muscular Dystrophy (DMD, BMD); Emery-Dreifuss muscular |
| | dystrophy (LMNA, LMN1, EMD2, FPLD, CMD1A, HGPS, |
| | LGMD1B, LMNA, LMN1, EMD2, FPLD, CMD1A); |
| | Facioscapulohumeral muscular dystrophy (FSHMD1A, |
| | FSHD1A); Muscular dystrophy (FKRP, MDC1C, LGMD2I, |
| | LAMA2, LAMM, LARGE, KIAA0609, MDC1D, FCMD, |
| | TTID, MYOT, CAPN3, CANP3, DYSF, LGMD2B, SGCG, |
| | LGMD2C, DMDA1, SCG3, SGCA, ADL, DAG2, LGMD2D, |
| | DMDA2, SGCB, LGMD2E, SGCD, SGD, LGMD2F, CMD1L, |
| | TCAP, LGMD2G, CMD1N, TRIM32, HT2A, LGMD2H, |
| | FKRP, MDC1C, LGMD2I, TTN, CMD1G, TMD, LGMD2J, |
| | POMT1, CAV3, LGMD1C, SEPN1, SELN, RSMD1, PLEC1, |
| | PLTN, EBS1); Osteopetrosis (LRP5, BMND1, LRP7, LR3, |
| | OPPG, VBCH2, CLCN7, CLC7, OPTA2, OSTM1, GL, |
| | TCIRG1, TIRC7, OC116, OPTB1); Muscular atrophy (VAPB, |
| | VAPC, ALS8, SMN1, SMA1, SMA2, SMA3, SMA4, BSCL2, |
| | SPG17, GARS, SMAD1, CMT2D, HEXB, IGHMBP2, |
| | SMUBP2, CATF1, SMARD1) |
| Dermatological diseases | Albinisim (TYR, OCA2, TYRP1, SLC45A2, LYST), |
| and disorders | Ectodermal dysplasias (EDAR, EDARADD, WNT10A), Ehlers- |
| | Danlos syndrome (COL5A1, COL5A2, COL1A1, COL1A2, |
| | COL3A1, TNXB, ADAMTS2, PLOD1, FKBP14), Ichthyosis- |
| | associated disorders (FLG, STS, TGM1, ALOXE3/ALOX12B, |
| | KRT1, KRT10, ABCA12, KRT2, GJB2, TGM1, ABCA12, |
| | CYP4F22, ALOXE3, CERS3, NSHDL, EBP, MBTPS2, GJB2, |
| | SPINK5, AGHD5, PHYH, PEX7, ALDH3A2, ERCC2, ERCC3, |
| | GFT2H5, GBA), Incontinentia pigmenti (IKBKG, NEMO), |
| | Tuberous sclerosis (TSC1, TSC2), Premature aging syndromes |
| | (POLR3A, PYCR1, LMA, POLD1, WRN, DMPK) |
| Neurological and Neuronal | ALS (SOD1, ALS2, STEX, FUS, TARDBP, VEGF (VEGF-a, |
| diseases and disorders | VEGF-b, VEGF-c); Alzheimer disease (APP, AAA, CVAP, |
| | AD1, APOE, AD2, PSEN2, AD4, STM2, APBB2, FE65L1, |
| | NOS3, PLAU, URK, ACE, DCP1, ACE1, MPO, PACIP1, |
| | PAXIP1L, PTIP, A2M, BLMH, BMH, PSEN1, AD3); Autism |
| | (Mecp2, BZRAP1, MDGA2, Sema5A, Neurexin 1, GLO1, |
| | MECP2, RTT, PPMX, MRX16, MRX79, NLGN3, NLGN4, |
| | KIAA1260, AUTSX2); Fragile X Syndrome (FMR2, FXR1, |
| | FXR2, mGLUR5); Huntington's disease and disease like |
| | disorders (HD, IT15, PRNP, PRIP, JPH3, JP3, HDL2, TBP, |
| | SCA17); Parkinson disease (NR4A2, NURR1, NOT, TINUR, |
| | SNCAIP, TBP, SCA17, SNCA, NACP, PARK1, PARK4, DJ1, |
| | PARK7, LRRK2, PARK8, PINK1, PARK6, UCHL1, PARK5, |
| | SNCA, NACP, PARK1, PARK4, PRKN, PARK2, PDJ, DBH, |
| | NDUFV2); Rett syndrome (MECP2, RTT, PPMX, MRX16, |
| | MRX79, CDKL5, STK9, MECP2, RTT, PPMX, MRX16, |
| | MRX79, x-Synuclein, DJ-1); Schizophrenia (Neuregulin1 |
| | (Nrg1), Erb4 (receptor for Neuregulin), Complexin1 (Cplx1), |
| | Tph1 Tryptophan hydroxylase, Tph2, Tryptophan hydroxylase 2, |
| | Neurexin 1, GSK3, GSK3a, GSK3b, 5-HTT (Slc6a4), COMT, |
| | DRD (Drd1a), SLC6A3, DAOA, DTNBP1, Dao (Dao1)); |
| | Secretase Related Disorders (APH-1 (alpha and beta), Presenilin |
| | (Psen1), nicastrin, (Ncstn), PEN-2, Nos1, Parp1, Natl, Nat2); |
| | Trinucleotide Repeat Disorders (HTT (Huntington's Dx), |
| | SBMA/SMAX1/AR (Kennedy's Dx), FXN/X25 (Friedrich's |
| | Ataxia), ATX3 (Machado-Joseph's Dx), ATXN1 and ATXN2 |
| | (spinocerebellar ataxias), DMPK (myotonic dystrophy), |
| | Atrophin-1 and Atn1 (DRPLA Dx), CBP (Creb-BP-global |
| | instability), VLDLR (Alzheimer's), Atxn7, Atxn10) |
| Ocular diseases and | Age-related macular degeneration (Abcr, Ccl2, Cc2, cp |
| disorders | (ceruloplasmin), Timp3, cathepsinD, Vldlr, Ccr2); Cataract |
| | (CRYAA, CRYA1, CRYBB2, CRYB2, PITX3, BFSP2, CP49, |
| | CP47, CRYAA, CRYA1, PAX6, AN2, MGDA, CRYBA1, |
| | CRYB1, CRYGC, CRYG3, CCL, LIM2, MP19, CRYGD, |
| | CRYG4, BFSP2, CP49, CP47, HSF4, CTM, HSF4, CTM, MIP, |
| | AQP0, CRYAB, CRYA2, CTPP2, CRYBB1, CRYGD, CRYG4, |
| | CRYBB2, CRYB2, CRYGC, CRYG3, CCL, CRYAA, CRYA1, |
| | GJA8, CX50, CAE1, GJA3, CX46, CZP3, CAE3, CCM1, CAM, |
| | KRIT1); Corneal clouding and dystrophy (APOA1, TGFBI, |
| | CSD2, CDGG1, CSD, BIGH3, CDG2, TACSTD2, TROP2, |
| | M1S1, VSX1, RINX, PPCD, PPD, KTCN, COL8A2, FECD, |
| | PPCD2, PIP5K3, CFD); Cornea plana congenital (KERA, |
| | CNA2); Glaucoma (MYOC, TIGR, GLC1A, JOAG, GPOA, |
| | OPTN, GLC1E, FIP2, HYPL, NRP, CYP1B1, GLC3A, OPA1, |
| | NTG, NPG, CYP1B1, GLC3A); Leber congenital |
| | amaurosis (CRB1, RP12, CRX, CORD2, CRD, RPGRIP1, |
| | LCA6, CORD9, RPE65, RP20, AIPL1, LCA4, GUCY2D, |
| | GUC2D, LCA1, CORD6, RDH12, LCA3); Macular dystrophy |
| | (ELOVL4, ADMD, STGD2, STGD3, RDS, RP7, PRPH2, |
| | PRPH, AVMD, AOFMD, VMD2) |

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of and any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a,” “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

It is understood that where a numerical range is recited herein, the present invention contemplates each integer between, and including, the upper and lower limits, unless otherwise stated.

In the description and claims of the present application, each of the verbs, “comprise,” “include” and “have” and conjugates thereof, are used to indicate that the object or objects of the verb are not necessarily a complete listing of components, elements or parts of the subject or subjects of the verb. Other terms as used herein are meant to be defined by their well-known meanings in the art.

The terms “polynucleotide”, “nucleotide”, “nucleotide sequence”, “nucleic acid” and “oligonucleotide” are used interchangeably. They refer to a polymeric form of nucleotides of any length, either deoxyribonueleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, in Irons, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers, A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

The term “nucleotide analog” or “modified nucleotide” refers to a nucleotide that contains one or more chemical modifications (e.g., substitutions), in or on the nitrogenous base of the nucleoside (e.g., cytosine (C), thymine (T) or uracil (U), adenine (A) or guanine (G)), in or on the sugar moiety of the nucleoside (e.g., ribose, deoxyribose, modified ribose, modified deoxyribose, six-membered sugar analog, or open-chain sugar analog), or the phosphate. Each of the RNA sequences described herein may comprise one or more nucleotide analogs.

As used herein, the following nucleotide identifiers are used to represent a referenced nucleotide base(s):

| Nucleotide | | | | |
|---|---|---|---|---|
| reference | Base(s) represented | | | |
| A | A | | | |
| C | | C | | |
| G | | | G | |
| T | | | | T |
| W | A | | | T |
| S | | C | G | |
| M | A | C | | |
| K | | | G | T |
| R | A | | G | |
| Y | | C | | T |
| B | | C | G | T |
| D | A | | G | T |
| H | A | C | | T |
| V | A | C | G | |
| N | A | C | G | T |

As used herein, the term “targeting sequence” or “targeting molecule” refers a nucleotide sequence or molecule comprising a nucleotide sequence that is capable of hybridizing to a specific target sequence, e.g., the targeting sequence has a nucleotide sequence which is at least partially complementary to the sequence being targeted along the length of the targeting sequence. The targeting sequence or targeting molecule may be part of a targeting RNA molecule that can form a complex with a CRISPR nuclease with the targeting sequence serving as the targeting portion of the CRISPR complex. When the molecule having the targeting sequence is present contemporaneously with the CRISPR molecule, the RNA molecule is capable of targeting the CRISPR nuclease to the specific target sequence. Each possibility represents a separate embodiment. A targeting RNA molecule can be custom designed to target any desired sequence.

The term “targets” as used herein, refers to preferential hybridization of a targeting sequence or a targeting molecule to a nucleic acid having a targeted nucleotide sequence. It is understood that the term “targets” encompasses variable hybridization efficiencies, such that there is preferential targeting of the nucleic acid having the targeted nucleotide sequence, but unintentional off-target hybridization in addition to on-target hybridization might also occur. It is understood that where an RNA molecule targets a sequence, a complex of the RNA molecule and a CRISPR nuclease molecule targets the sequence for nuclease activity.

In the context of targeting a DNA sequence that is present in a plurality of cells, it is understood that the targeting encompasses hybridization of the guide sequence portion of the RNA molecule with the sequence in one or more of the cells, and also encompasses hybridization of the RNA molecule with the target sequence in fewer than all of the cells in the plurality of cells. Accordingly, it is understood that where an RNA molecule targets a sequence in a plurality of cells, a complex of the RNA molecule and a CRISPR nuclease is understood to hybridize with the target sequence in one or more of the cells, and also may hybridize with the target sequence in fewer than all of the cells. Accordingly, it is understood that the complex of the RNA molecule and the CRISPR nuclease introduces a double strand break in relation to hybridization with the target sequence in one or more cells and may also introduce a double strand break in relation to hybridization with the target sequence in fewer than all of the cells. As used herein, the term “modified cells” refers to cells in which a double strand break is affected by a complex of an RNA molecule and the CRISPR nuclease as a result of hybridization with the target sequence, i.e. on-target hybridization.

As used herein the term “wild type” is a term of the art understood by skilled persons and means the typical form of an organism, strain, gene or characteristic as it occurs in nature as distinguished from mutant or variant forms. Accordingly, as used herein, where a sequence of amino acids or nucleotides refers to a wild type sequence, a variant refers to variant of that sequence, e.g., comprising substitutions, deletions, insertions. In embodiments of the present invention, an engineered CRISPR nuclease is a variant CRISPR nuclease comprising at least one amino acid modification (e.g., substitution, deletion, and/or insertion) compared to the CRISPR nuclease of any of the CRISPR nucleases indicated in Table 1.

The terms “non-naturally occurring” or “engineered” are used interchangeably and indicate human manipulation. The terms, when referring to nucleic acid molecules or polypeptides may mean that the nucleic acid molecule or the polypeptide is at least substantially free from at least one other component with which they are naturally associated in nature and as found in nature.

As used herein the term “amino acid” includes natural and/or unnatural or synthetic amino acids, including glycine and both the D or I, optical isomers, and amino acid analogs and peptidomimetics.

As used herein, “genomic DNA” refers to linear and/or chromosomal DNA and/or to plasmid or other extrachromosomal DNA sequences present in the cell or cells of interest. In some embodiments, the cell of interest is a eukaryotic cell. In some embodiments, the cell of interest is a prokaryotic cell. In some embodiments, the methods produce double-stranded breaks (DSBs) at pre-determined target sites in a genomic DNA sequence, resulting in mutation, insertion, and/or deletion of DNA sequences at the target site(s) in a genome.

“Eukaryotic” cells include, but are not limited to, fungal cells (such as yeast), plant cells, animal cells, mammalian cells and human cells.

The term “nuclease” as used herein refers to an enzyme capable of cleaving the phosphodiester bonds between the nucleotide subunits of nucleic acid. A nuclease may be isolated or derived from a natural source. The natural source may be any living organism. Alternatively, a nuclease may be a modified or a synthetic protein which retains the phosphodiester bond cleaving activity.

The term “PAM” as used herein refers to a nucleotide sequence of a target DNA located in proximity to the targeted DNA sequence and recognized by the CRISPR nuclease. The PAM sequence may differ depending on the nuclease identity.

The term “mutation disorder” or “mutation disease” as used herein refers to any disorder or disease that is related to dysfunction of a gene caused by a mutation. A dysfunctional gene manifesting as a mutation disorder contains a mutation in at least one of its alleles and is referred to as a “disease-associated gene.” The mutation may be in any portion of the disease-associated gene, for example, in a regulatory, coding, or non-coding portion. The mutation may be any class of mutation, such as a substitution, insertion, or deletion. The mutation of the disease-associated gene may manifest as a disorder or disease according to the mechanism of any type of mutation, such as a recessive, dominant negative, gain-of-function, loss-of-function, or a mutation leading to haploinsufficiency of a gene product.

A skilled artisan will appreciate that embodiments of the present invention disclose RNA molecules capable of complexing with a nuclease, e.g. a CRISPR nuclease, such as to associate with a target genomic DNA sequence of interest next to a protospacer adjacent motif (PAM). The nuclease then mediates cleavage of target DNA to create a double-stranded break within the protospacer.

In embodiments of the present invention, a CRISPR nuclease and a targeting molecule form a CRISPR complex that binds to a target DNA sequence to effect cleavage of the target DNA sequence. A CRISPR nuclease may form a CRISPR complex comprising the CRISPR nuclease and RNA molecule without a further, separate tracrRNA molecule. Alternatively, CRISPR nucleases may form a CRISPR complex between the CRISPR nuclease, an RNA molecule, and a tracrRNA molecule.

The term “protein binding sequence” or “nuclease binding sequence” refers to a sequence capable of binding with a CRISPR nuclease to form a CRISPR complex. A skilled artisan will understand that a tracrRNA capable of binding with a CRISPR nuclease to form a CRISPR complex comprises a protein or nuclease binding sequence.

An “RNA binding portion” of a CRISPR nuclease refers to a portion of the CRISPR nuclease which may bind to an RNA molecule to form a CRISPR complex, e.g. the nuclease binding sequence of a tracrRNA molecule. An “activity portion” or “active portion” of a CRISPR nuclease refers to a portion of the CRISPR nuclease which effects a double strand break in a DNA molecule, for example when in complex with a DNA-targeting RNA molecule.

An RNA molecule may comprise a sequence sufficiently complementary to a tracrRNA molecule so as to hybridize to the tracrRNA via basepairing and promote the formation of a CRISPR complex. (See U.S. Pat. No. 8,906,616). In embodiments of the present invention, the RNA molecule may further comprise a portion having a tracr mate sequence.

In embodiments of the present invention, the targeting molecule may further comprise the sequence of a tracrRNA molecule. Such embodiments may be designed as a synthetic fusion of the guide portion of the RNA molecule (gRNA or crRNA) and the trans-activating crRNA (tracrRNA), together forming a single guide RNA (sgRNA). (See Jinek et al., Science (2012)). Embodiments of the present invention may also form CRISPR complexes utilizing a separate tracrRNA molecule and a separate RNA molecule comprising a guide sequence portion. In such embodiments the tracrRNA molecule may hybridize with the RNA molecule via base pairing and may be advantageous in certain applications of the invention described herein.

In embodiments of the present invention an RNA molecule may comprise a “nexus” region and/or “hairpin” regions which may further define the structure of the RNA molecule. (See Briner et al., Molecular Cell (2014)).

As used herein, the term “direct repeat sequence” refers to two or more repeats of a specific amino acid sequence of nucleotide sequence.

As used herein, an RNA sequence or molecule capable of “interacting with” or “binding” with a CRISPR nuclease refers to the RNA sequence or molecules ability to form a CRISPR complex with the CRISPR nuclease.

As used herein, the term “operably linked” refers to a relationship (i.e. fusion, hybridization) between two sequences or molecules permitting them to function in their intended manner. In embodiments of the present invention, when an RNA molecule is operably linked to a promoter, both the RNA molecule and the promotor are permitted to function in their intended manner.

As used herein, the term “heterologous promoter” refers to a promoter that does not naturally occur together with the molecule or pathway being promoted.

As used herein, a sequence or molecule has an X % “sequence identity” to another sequence or molecule if X % of bases or amino acids between the sequences of molecules are the same and in the same relative position. For example, a first nucleotide sequence having at least a 95% sequence identity with a second nucleotide sequence will have at least 95% of bases, in the same relative position, identical with the other sequence.

### Nuclear Localization Sequences

The terms “nuclear localization sequence” and “NLS” are used interchangeably to indicate an amino acid sequence/peptide that directs the transport of a protein with which it is associated from the cytoplasm of a cell across the nuclear envelope barrier. The term “NLS” is intended to encompass not only the nuclear localization sequence of a particular peptide, but also derivatives thereof that are capable of directing translocation of a cytoplasmic polypeptide across the nuclear envelope barrier. NLSs are capable of directing nuclear translocation of a polypeptide when attached to the N-terminus, the C-terminus, or both the N- and C-termini of the polypeptide. In addition, a polypeptide having an NLS coupled by its N- or C-terminus to amino acid side chains located randomly along the amino acid sequence of the polypeptide will be translocated. Typically, an NLS consists of one or more short sequences of positively charged lysines or arginines exposed on the protein surface, but other types of NLS are known. Non-limiting examples of NLSs include an NLS sequence derived from: the SV40 virus large T-antigen, nucleoplasmin, c-myc, the hRNPA1 M9 NLS, the IBB domain from importin-alpha, myoma T protein, human p53, mouse c-abl IV, influenza vims NS1, Hepatitis virus delta antigen, mouse Mx 1 protein, human poly(ADP-ribose) polymerase, and the steroid hormone receptors (human) glucocorticoid. Such NLS sequences are listed as SEQ ID NOs: 69-84.

### Delivery

The CRISPR nuclease or CRISPR compositions described herein may be delivered as a protein, DNA molecules, RNA molecules, Ribonucleoproteins (RNP), nucleic acid vectors, or any combination thereof. In some embodiments, the RNA molecule comprises a chemical modification. Non-limiting examples of suitable chemical modifications include 2′-0-methyl (M), 2′-0-methyl, 3′phosphorothioate (MS) or 2′-0-methyl, 3′thioPACE (MSP), pseudouridine, and 1-methyl pseudo-uridine. Each possibility represents a separate embodiment of the present invention.

The CRISPR nucleases and/or polynucleotides encoding same described herein, and optionally additional proteins (e.g., ZFPs, TALENs, transcription factors, restriction enzymes) and/or nucleotide molecules such as guide RNA may be delivered to a target cell by any suitable means. The target cell may be any type of cell e.g., eukaryotic or prokaryotic, in any environment e.g., isolated or not, maintained in culture, in vitro, ex vivo, in vivo or in planta.

In some embodiments, the composition to be delivered includes mRNA of the nuclease and RNA of the guide. In some embodiments, the composition to be delivered includes mRNA of the nuclease, RNA of the guide and a donor template. In some embodiments, the composition to be delivered includes the CRISPR nuclease and guide RNA. In some embodiments, the composition to be delivered includes the CRISPR nuclease, guide RNA and a donor template for gene editing via, for example, homology directed repair. In some embodiments, the composition to be delivered includes mRNA of the nuclease, DNA-targeting RNA and the tracrRNA. In some embodiments, the composition to be delivered includes mRNA of the nuclease, DNA-targeting RNA and the tracrRNA and a donor template. In some embodiments, the composition to be delivered includes the CRISPR nuclease DNA-targeting RNA and the tracrRNA. In some embodiments, the composition to be delivered includes the CRISPR nuclease, DNA-targeting RNA and the tracrRNA and a donor template for gene editing via, for example, homology directed repair.

Any suitable viral vector system may be used to deliver RNA compositions. Conventional viral and non-viral based gene transfer methods can be used to introduce nucleic acids and/or CRISPR nuclease in cells (e.g., mammalian cells, plant cells, etc.) and target tissues. Such methods can also be used to administer nucleic acids encoding and/or CRISPR nuclease protein to cells in vitro. In certain embodiments, nucleic acids and/or CRISPR nuclease are administered for in vivo or ex vivo gene therapy uses. Non-viral vector delivery systems include naked nucleic acid, and nucleic acid complexed with a delivery vehicle such as a liposome or poloxamer. For a review of gene therapy procedures, see Anderson, Science (1992); Nabel and Felgner, TIBTECH (1993); Mitani and Caskey, TIBTECH (1993); Dillon, TIBTECH (1993); Miller, Nature (1992); Van Brunt, Biotechnology (1988); Vigne et al., Restorative Neurology and Neuroscience 8:35-36 (1995); Kremer and Perricaudet, British Medical Bulletin (1995); Haddada et al., Current Topics in Microbiology and Immunology (1995); and Yu et al., Gene Therapy 1:13-26 (1994).

Methods of non-viral delivery of nucleic acids and/or proteins include electroporation, lipofection, microinjection, biolistics, particle gun acceleration, virosomes, liposomes, immunoliposomes, polycation or lipid:nucleic acid conjugates, artificial virions, and agent-enhanced uptake of nucleic acids or can be delivered to plant cells by bacteria or viruses (e.g., Agrobacterium, Rhizobium sp. NGR234, Sinorhizoboiummeliloti, Mesorhizobium loti, tobacco mosaic virus, potato virus X, cauliflower mosaic virus and cassava vein mosaic virus. See, e.g., Chung et al. Trends Plant Sci. (2006). Sonoporation using, e.g., the Sonitron 2000 system (Rich-Mar) can also be used for delivery of nucleic acids. Cationic-lipid mediated delivery of proteins and/or nucleic acids is also contemplated as an in vivo or in vitro delivery method. See Zuris et al., Nat. Biotechnol. (2015), Coelho et al., N. Engl. J. Med. (2013); Judge et al., Mol. Ther. (2006); and Basha et al., Mol. Ther. (2011).

Additional exemplary nucleic acid delivery systems include those provided by Amaxa® Biosystems (Cologne, Germany), Maxcyte, Inc. (Rockville, Md.), BTX Molecular Delivery Systems (Holliston, Mass.) and Copernicus Therapeutics Inc., (see for example U.S. Pat. No. 6,008,336). Lipofection is described in e.g., U.S. Pat. Nos. 5,049,386, 4,946,787; and 4,897,355) and lipofection reagents are sold commercially (e.g., Transfectam.™., Lipofectin.™. and Lipofectamine.™. RNAiMAX). Cationic and neutral lipids that are suitable for efficient receptor-recognition lipofection of polynucleotides include those disclosed in PCT International Publication Nos. WO/1991/017424 and WO/1991/016024. Delivery can be to cells (ex vivo administration) or target tissues (in vivo administration).

The preparation of lipid:nucleic acid complexes, including targeted liposomes such as immunolipid complexes, is well known to one of skill in the art (see, e.g., Crystal, Science (1995); Blaese et al., Cancer Gene Ther. (1995); Behr et al., Bioconjugate Chem. (1994); Remy et al., Bioconjugate Chem. (1994); Gao and Huang, Gene Therapy (1995); Ahmad and Allen, Cancer Res., (1992); U.S. Pat. Nos. 4,186,183; 4,217,344; 4,235,871; 4,261,975; 4,485,054; 4,501,728; 4,774,085; 4,837,028; and 4,946,787).

Additional methods of delivery include the use of packaging the nucleic acids to be delivered into EnGeneIC delivery vehicles (EDVs). These EDVs are specifically delivered to target tissues using bispecific antibodies where one arm of the antibody has specificity for the target tissue and the other has specificity for the EDV. The antibody brings the EDVs to the target cell surface and then the EDV is brought into the cell by endocytosis. Once in the cell, the contents are released (see MacDiamid et al., Nature Biotechnology (2009)).

The use of RNA or DNA viral based systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of nucleic acids include, but are not limited to, recombinant retroviral, lentivirus, adenoviral, adeno-associated, vaccinia and herpes simplex virus vectors for gene transfer. However, an RNA virus is preferred for delivery of the RNA compositions described herein. Additionally, high transduction efficiencies have been observed in many different cell types and target tissues. Nucleic acid of the invention may be delivered by non-integrating lentivirus. Optionally, RNA delivery with Lentivirus is utilized. Optionally the lentivirus includes mRNA of the nuclease, RNA of the guide. Optionally the lentivirus includes mRNA of the nuclease, RNA of the guide and a donor template. Optionally, the lentivirus includes the nuclease protein, guide RNA. Optionally, the lentivirus includes the nuclease protein, guide RNA and/or a donor template for gene editing via, for example, homology directed repair. Optionally the lentivirus includes mRNA of the nuclease, DNA-targeting RNA, and the tracrRNA. Optionally the lentivirus includes mRNA of the nuclease, DNA-targeting RNA, and the tracrRNA, and a donor template. Optionally, the lentivirus includes the nuclease protein, DNA-targeting RNA, and the tracrRNA. Optionally, the lentivirus includes the nuclease protein, DNA-targeting RNA, and the tracrRNA, and a donor template for gene editing via, for example, homology directed repair.

As mentioned above, the compositions described herein may be delivered to a target cell using a non-integrating lentiviral particle method, e.g. a LentiFlash® system. Such a method may be used to deliver mRNA or other types of RNAs into the target cell, such that delivery of the RNAs to the target cell results in assembly of the compositions described herein inside of the target cell. See also PCT International Publication Nos. WO2013/014537, WO2014/016690, WO2016185125, WO2017194902, and WO2017194903.

The tropism of a retrovirus can be altered by incorporating foreign envelope proteins, expanding the potential target population of target cells. Lentiviral vectors are retroviral vectors capable of transducing or infecting non-dividing cells and typically produce high viral titers. Selection of a retroviral gene transfer system depends on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immunodeficiency virus (SIV), human immunodeficiency virus (HIV), and combinations thereof (see, e.g., Buchscher Panganiban, J. Virol. (1992); Johann et al., J. Virol. (1992); Sommerfelt et al., Virol. (1990); Wilson et al., J. Virol. (1989); Miller et al., J. Virol. (1991); PCT International Publication No. WO/1994/026877A1).

At least six viral vector approaches are currently available for gene transfer in clinical trials, which utilize approaches that involve complementation of defective vectors by genes inserted into helper cell lines to generate the transducing agent.

pLASN and MFG-S are examples of retroviral vectors that have been used in clinical trials (Dunbar et al., Blood (1995); Kohn et al., Nat. Med. (1995); Malech et al., PNAS (1997)). PA317/pLASN was the first therapeutic vector used in a gene therapy trial. (Blaese et al., Science (1995)). Transduction efficiencies of 50% or greater have been observed for MFG-S packaged vectors. (Ellem et al., Immunol Immunother. (1997); Dranoff et al., Hum. Gene Ther. (1997).

Packaging cells are used to form virus particles that are capable of infecting a host cell. Such cells include 293 cells, which package adenovirus, AAV, and psi.2 cells or PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host (if applicable), other viral sequences being replaced by an expression cassette encoding the protein to be expressed. The missing viral functions are supplied in trans by the packaging cell line. For example, AAV vectors used in gene therapy typically only possess inverted terminal repeat (ITR) sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additionally, AAV can be produced at clinical scale using baculovirus systems (see U.S. Pat. No. 7,479,554).

In many gene therapy applications, it is desirable that the gene therapy vector be delivered with a high degree of specificity to a particular tissue type. Accordingly, a viral vector can be modified to have specificity for a given cell type by expressing a ligand as a fusion protein with a viral coat protein on the outer surface of the virus. The ligand is chosen to have affinity for a receptor known to be present on the cell type of interest. For example, Han et al., Proc. Natl. Acad. Sci. USA (1995), reported that Moloney murine leukemia virus can be modified to express human heregulin fused to gp70, and the recombinant virus infects certain human breast cancer cells expressing human epidermal growth factor receptor. This principle can be extended to other virus-target cell pairs, in which the target cell expresses a receptor and the virus expresses a fusion protein comprising a ligand for the cell-surface receptor. For example, filamentous phage can be engineered to display antibody fragments (e.g., FAB or Fv) having specific binding affinity for virtually any chosen cellular receptor. Although the above description applies primarily to viral vectors, the same principles can be applied to non-viral vectors. Such vectors can be engineered to contain specific uptake sequences which favor uptake by specific target cells.

Gene therapy vectors can be delivered in vivo by administration to an individual patient, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, usually after selection for cells which have incorporated the vector. In some embodiments, delivery of mRNA in-vivo and ex-vivo, and RNPs delivery may be utilized.

Ex vivo cell transfection for diagnostics, research, or for gene therapy (e.g., via re-infusion of the transfected cells into the host organism) is well known to those of skill in the art. In a preferred embodiment, cells are isolated from the subject organism, transfected with an RNA composition, and re-infused back into the subject organism (e.g., patient). Various cell types suitable for ex vivo transfection are well known to those of skill in the art (see, e.g., Freshney, “Culture of Animal Cells, A Manual of Basic Technique and Specialized Applications (6th edition, 2010)) and the references cited therein for a discussion of how to isolate and culture cells from patients).

Suitable cells include but not limited to eukaryotic and prokaryotic cells and/or cell lines. Non-limiting examples of such cells or cell lines generated from such cells include COS, CHO (e.g., CHO-S, CHO-K1, CHO-DG44, CHO-DUXB11, CHO-DUKX, CHOK1SV), VERO, MDCK, WI38, V79, B14AF28-G3, BHK, HaK, NSO, SP2/0-Ag14, HeLa, HEK293 (e.g., HEK293-F, HEK293-H, HEK293-T), and perC6 cells, any plant cell (differentiated or undifferentiated) as well as insect cells such as Spodopterafugiperda (Sf), or fungal cells such as Saccharomyces, Pichia and Schizosaccharomyces. In certain embodiments, the cell line is a CHO-K1, MDCK or HEK293 cell line. Additionally, primary cells may be isolated and used ex vivo for reintroduction into the subject to be treated following treatment with the nucleases (e.g. ZFNs or TALENs) or nuclease systems (e.g. CRISPR). Suitable primary cells include peripheral blood mononuclear cells (PBMC), and other blood cell subsets such as, but not limited to, CD4+ T cells or CD8+ T cells. Suitable cells also include stem cells such as, by way of example, embryonic stem cells, induced pluripotent stem cells, hematopoietic stem cells (CD34+), neuronal stem cells and mesenchymal stem cells.

In one embodiment, stem cells are used in ex vivo procedures for cell transfection and gene therapy. The advantage to using stem cells is that they can be differentiated into other cell types in-vitro or can be introduced into a mammal (such as the donor of the cells) where they will engraft in the bone marrow. Methods for differentiating CD34+ cells in vitro into clinically important immune cell types using cytokines such a GM-CSF, IFN-gamma. and TNF-alpha are known (as a non-limiting example see, Inaba et al., J. Exp. Med. (1992)).

Stem cells are isolated for transduction and differentiation using known methods. For example, stem cells are isolated from bone marrow cells by panning the bone marrow cells with antibodies which bind unwanted cells, such as CD4+ and CD8+(T cells), CD45+(panB cells), GR-1 (granulocytes), and Iad (differentiated antigen presenting cells) (as a non-limiting example see Inaba et al., J. Exp. Med. (1992)). Stem cells that have been modified may also be used in some embodiments.

Notably, the CRISPR nuclease described herein may be suitable for genome editing in post-mitotic cells or any cell which is not actively dividing, e.g., arrested cells. Examples of post-mitotic cells which may be edited using a CRISPR nuclease of the present invention include, but are not limited to, myocyte, a cardiomyocyte, a hepatocyte, an osteocyte and a neuron.

Vectors (e.g., retroviruses, liposomes, etc.) containing therapeutic RNA compositions can also be administered directly to an organism for transduction of cells in vivo. Alternatively, naked RNA or mRNA can be administered. Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art, and, although more than one route can be used to administer a particular composition, a particular route can often provide a more immediate and more effective reaction than another route.

Vectors suitable for introduction of transgenes into immune cells (e.g., T-cells) include non-integrating lentivirus vectors. See, for example, U.S. Patent Publication No. 2009/0117617.

Pharmaceutically acceptable carriers are determined in part by the particular composition being administered, as well as by the particular method used to administer the composition. Accordingly, there is a wide variety of suitable formulations of pharmaceutical compositions available, as described below (see, e.g., Remington's Pharmaceutical Sciences, 17th ed., 1989).

### DNA Repair by Homologous Recombination

The term “homology-directed repair” or “HDR” refers to a mechanism for repairing DNA damage in cells, for example, during repair of double-stranded and single-stranded breaks in DNA. HDR requires nucleotide sequence homology and uses a “nucleic acid template” (nucleic acid template or donor template used interchangeably herein) to repair the sequence where the double-stranded or single break occurred (e.g., DNA target sequence). This results in the transfer of genetic information from, for example, the nucleic acid template to the DNA target sequence. HDR may result in alteration of the DNA target sequence (e.g., insertion, deletion, mutation) if the nucleic acid template sequence differs from the DNA target sequence and part or all of the nucleic acid template polynucleotide or oligonucleotide is incorporated into the DNA target sequence. In some embodiments, an entire nucleic acid template polynucleotide, a portion of the nucleic acid template polynucleotide, or a copy of the nucleic acid template is integrated at the site of the DNA target sequence.

The terms “nucleic acid template” and “donor”, refer to a nucleotide sequence that is inserted or copied into a genome. The nucleic acid template comprises a nucleotide sequence, e.g., of one or more nucleotides, that will be added to or will template a change in the target nucleic acid or may be used to modify the target sequence. A nucleic acid template sequence may be of any length, for example between 2 and 10,000 nucleotides in length (or any integer value there between or there above), preferably between about 100 and 1,000 nucleotides in length (or any integer there between), more preferably between about 200 and 500 nucleotides in length. A nucleic acid template may be a single stranded nucleic acid, a double stranded nucleic acid. In some embodiment, the nucleic acid template comprises a nucleotide sequence, e.g., of one or more nucleotides, that corresponds to wild type sequence of the target nucleic acid, e.g., of the target position. In some embodiment, the nucleic acid template comprises a ribonucleotide sequence, e.g., of one or more ribonucleotides, that corresponds to wild type sequence of the target nucleic acid, e.g., of the target position. In some embodiment, the nucleic acid template comprises modified ribonucleotides.

Insertion of an exogenous sequence (also called a “donor sequence,” donor template” or “donor”), for example, for correction of a mutant gene or for increased expression of a wild-type gene can also be carried out. It will be readily apparent that the donor sequence is typically not identical to the genomic sequence where it is placed. A donor sequence can contain a non-homologous sequence flanked by two regions of homology to allow for efficient HDR at the location of interest. Additionally, donor sequences can comprise a vector molecule containing sequences that are not homologous to the region of interest in cellular chromatin. A donor molecule can contain several, discontinuous regions of homology to cellular chromatin. For example, for targeted insertion of sequences not normally present in a region of interest, said sequences can be present in a donor nucleic acid molecule and flanked by regions of homology to sequence in the region of interest.

The donor polynucleotide can be DNA or RNA, single-stranded and/or double-stranded and can be introduced into a cell in linear or circular form. See, e.g., U.S. Patent Publication Nos. 2010/0047805; 2011/0281361; 2011/0207221; and 2019/0330620. If introduced in linear form, the ends of the donor sequence can be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang and Wilson, Proc. Natl. Acad. Sci. USA (1987); Nehls et al., Science (1996). Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues.

Accordingly, embodiments of the present invention using a donor template for repair may use a DNA or RNA, single-stranded and/or double-stranded donor template that can be introduced into a cell in linear or circular form. In embodiments of the present invention a gene-editing composition comprises: (1) an RNA molecule comprising a guide sequence to affect a double strand break in a gene prior to repair and (2) a donor RNA template for repair, the RNA molecule comprising the guide sequence is a first RNA molecule and the donor RNA template is a second RNA molecule. In some embodiments, the guide RNA molecule and template RNA molecule are connected as part of a single molecule.

A donor sequence may also be an oligonucleotide and be used for gene correction or targeted alteration of an endogenous sequence. The oligonucleotide may be introduced to the cell on a vector, may be electroporated into the cell, or may be introduced via other methods known in the art. The oligonucleotide can be used to ‘correct’ a mutated sequence in an endogenous gene (e.g., the sickle mutation in beta globin), or may be used to insert sequences with a desired purpose into an endogenous locus.

A polynucleotide can be introduced into a cell as part of a vector molecule having additional sequences such as, for example, replication origins, promoters and genes encoding antibiotic resistance. Moreover, donor polynucleotides can be introduced as naked nucleic acid, as nucleic acid complexed with an agent such as a liposome or poloxamer, or can be delivered by recombinant viruses (e.g., adenovirus, AAV, herpesvirus, retrovirus, lentivirus and integrase defective lentivirus (IDLY)).

The donor is generally inserted so that its expression is driven by the endogenous promoter at the integration site, namely the promoter that drives expression of the endogenous gene into which the donor is inserted. However, it will be apparent that the donor may comprise a promoter and/or enhancer, for example a constitutive promoter or an inducible or tissue specific promoter.

The donor molecule may be inserted into an endogenous gene such that all, some or none of the endogenous gene is expressed. For example, a transgene as described herein may be inserted into an endogenous locus such that some (N-terminal and/or C-terminal to the transgene) or none of the endogenous sequences are expressed, for example as a fusion with the transgene. In other embodiments, the transgene (e.g., with or without additional coding sequences such as for the endogenous gene) is integrated into any endogenous locus, for example a safe-harbor locus, for example a CCR5 gene, a CXCR4 gene, a PPP1R12c (also known as AAVS1) gene, an albumin gene or a Rosa gene. See, e.g., U.S. Pat. Nos. 7,951,925 and 8,110,379; U.S. Publication Nos. 2008/0159996; 20100/0218264; 2010/0291048; 2012/0017290; 2011/0265198; 2013/0137104; 2013/0122591; 2013/0177983 and 2013/0177960 and U.S. Provisional Application No. 61/823,689).

When endogenous sequences (endogenous or part of the transgene) are expressed with the transgene, the endogenous sequences may be full-length sequences (wild-type or mutant) or partial sequences. Preferably the endogenous sequences are functional. Non-limiting examples of the function of these full length or partial sequences include increasing the serum half-life of the polypeptide expressed by the transgene (e.g., therapeutic gene) and/or acting as a carrier.

Furthermore, although not required for expression, exogenous sequences may also include transcriptional or translational regulatory sequences, for example, promoters, enhancers, insulators, internal ribosome entry sites, sequences encoding 2A peptides and/or polyadenylation signals.

In certain embodiments, the donor molecule comprises a sequence selected from the group consisting of a gene encoding a protein (e.g., a coding sequence encoding a protein that is lacking in the cell or in the individual or an alternate version of a gene encoding a protein), a regulatory sequence and/or a sequence that encodes a structural nucleic acid such as a microRNA or siRNA.

For the foregoing embodiments, each embodiment disclosed herein is contemplated as being applicable to each of the other disclosed embodiment. For example, it is understood that any of the RNA molecules or compositions of the present invention may be utilized in any of the methods of the present invention.

As used herein, all headings are simply for organization and are not intended to limit the disclosure in any manner. The content of any individual section may be equally applicable to all sections.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Generally, the nomenclature used herein, and the laboratory procedures utilized in the present invention include molecular, biochemical, microbiological and recombinant DNA techniques. Such techniques are thoroughly explained in the literature. See, for example, Sambrook et al., “Molecular Cloning: A laboratory Manual” (1989); Ausubel, R. M. (Ed.), “Current Protocols in Molecular Biology” Volumes I-III (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (Eds.), “Genome Analysis: A Laboratory Manual Series”, Vols. 1-4, Cold Spring Harbor Laboratory Press, New York (1998); Methodologies as set forth in U.S. Pat. Nos. 4,666,828; 4,683,202; 4,801,531; 5,192,659 and 5,272,057; Cellis, J. E. (Ed.), “Cell Biology: A Laboratory Handbook”, Volumes I-III (1994); Freshney, “Culture of Animal Cells—A Manual of Basic Technique” Third Edition, Wiley-Liss, N. Y. (1994); Coligan J. E. (Ed.), “Current Protocols in Immunology” Volumes I-III (1994); Stites et al. (Eds.), “Basic and Clinical Immunology” (8th Edition), Appleton & Lange, Norwalk, C T (1994); Mishell and Shiigi (Eds.), “Strategies for Protein Purification and Characterization—A Laboratory Course Manual” CSHL Press (1996); Clokie and Kropinski (Eds.), “Bacteriophage Methods and Protocols”, Volume 1: Isolation, Characterization, and Interactions (2009), all of which are incorporated by reference. Other general references are provided throughout this document.

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only.

### EXPERIMENTAL DETAILS

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only.

CRISPR repeat (crRNA), transactivating crRNA (tracrRNA), nuclease polypeptide, and PAM sequences were predicted from different metagenomic databases of sequences of environmental samples. The bacterial species/strain from which the CRISPR repeat, tracRNA sequence, and nuclease polypeptide sequence were predicted is provided in Table 1.

### Construction of OMNI-50 Nuclease Polypeptides

For construction of OMNI-50 nuclease polypeptides, the open reading frame of the OMNI-50 nuclease was codon optimized for human cell line expression. The optimized ORF was cloned into the bacterial plasmid pb-NNC and into the mammalian plasmid pmOMNI (Table 4).

Prediction and Construction of sgRNA

For the OMNI-50 nuclease, the sgRNA was predicted by detection of the CRISPR repeat array sequence (crRNA) and a trans-activating crRNA (tracrRNA) in the bacterial genome in which the nuclease was identified. The native pre-mature crRNA and tracrRNA sequences were connected in-silico with tetra-loop ‘gaaa’ and the secondary structure elements of the duplex were predicted by using an RNA secondary structure prediction tool.

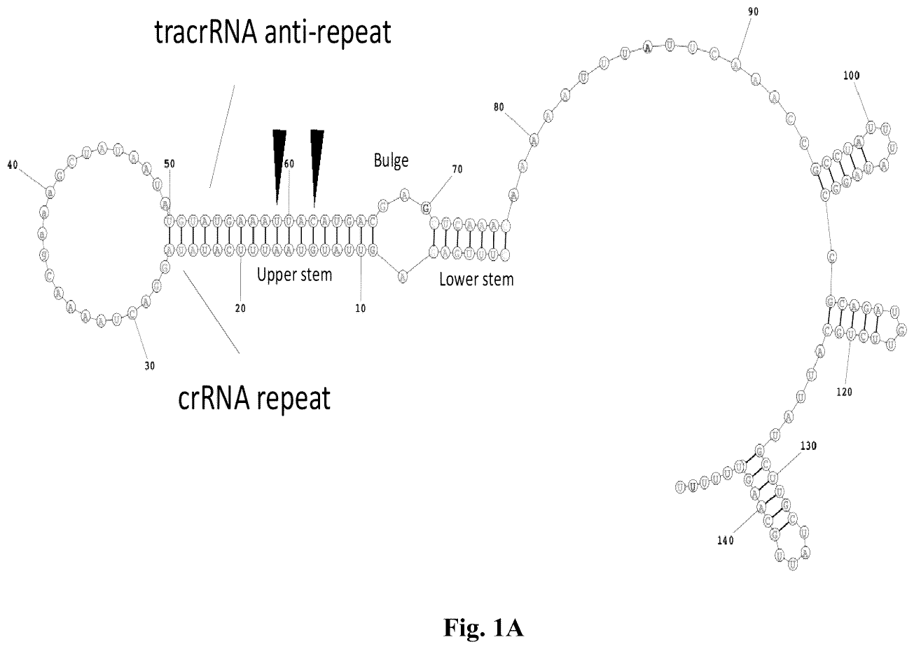

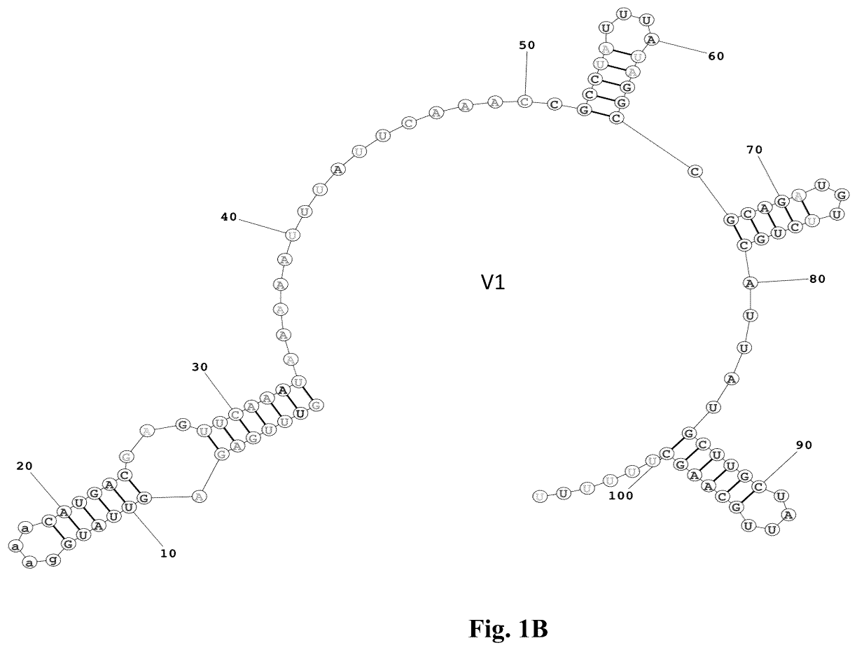

The predicted secondary structures of the full duplex RNA elements (i.e. crRNA-tracrRNA chimera) was used for identification of possible tracr sequences for the design of a sgRNA having various versions for the OMNI-50 nuclease (see for example, FIG. 1A). By shortening the duplex at the upper stem at different locations, the crRNA and tracrRNA were connected with tetra-loop ‘gaaa', thereby generating possible sgRNA scaffolds (see for example, FIG. 1B; OMNI-50 sgRNA designs are listed in Table 2). At least two versions of possible designed scaffolds for OMNI-50 were synthesized and connected downstream to a 22 nt universal unique spacer sequence (T2, SEQ ID NO: 56), and cloned into a bacterial expression plasmid under a constitutive promoter and into a mammalian expression plasmid under a U6 promoter (pbGuide and pmGuide, respectively, Table 4).

In order to overcome potential transcriptional and structural constraints and to assess the plasticity of the sgRNA scaffold in the human cellular environmental context, several versions of the sgRNA were tested. In each case the modifications represent small variations in the nucleotide sequence of the possible sgRNA (FIG. 1C, Table 2)

(SEQ ID NO: 55)
T1 - GGTGCGGTTCACCAGGGTGTCG
(SEQ ID NO: 56)
T2 - GGAAGAGCAGAGCCTTGGTCTC

### In-Vitro Depletion Assay by TXTL

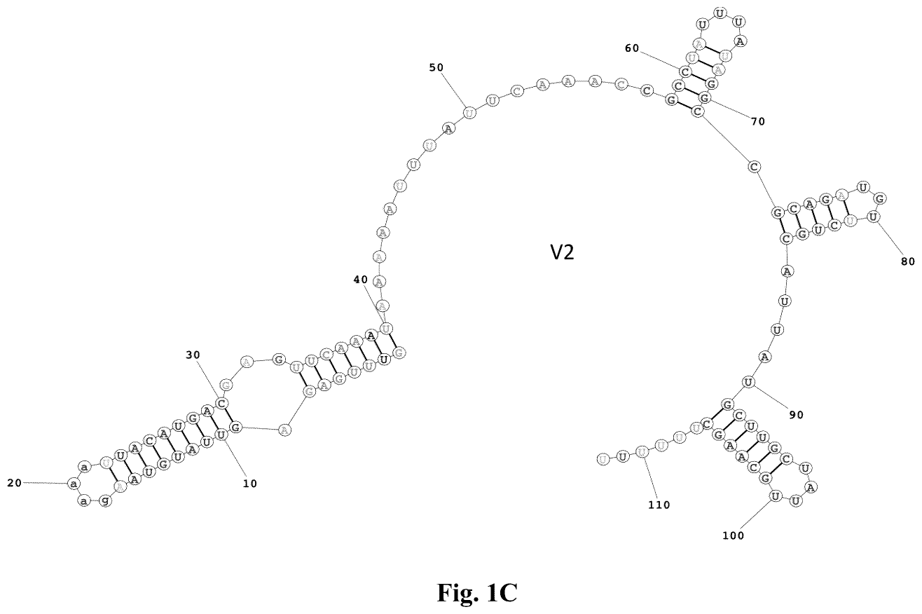

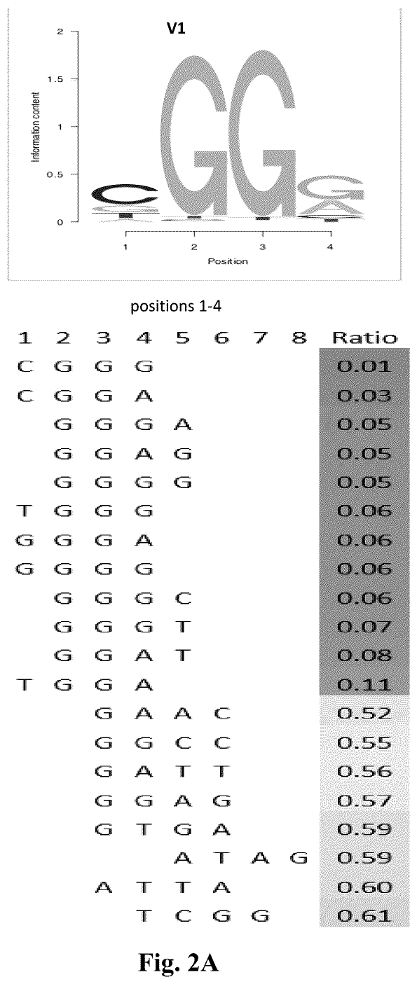

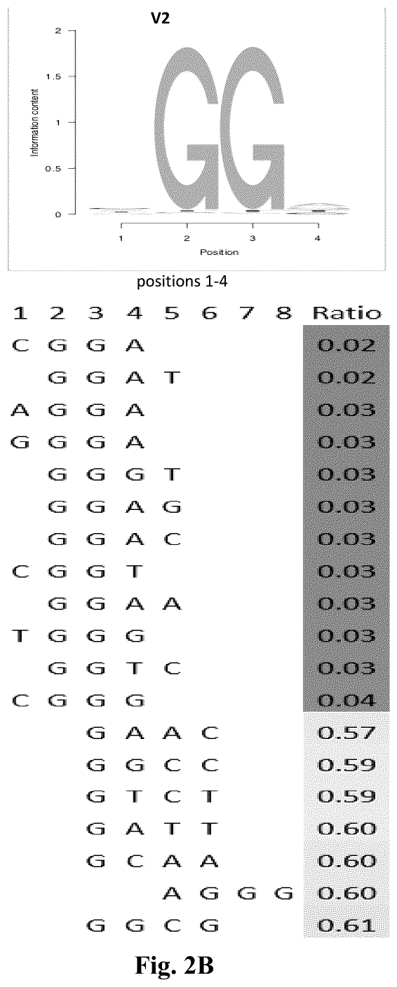

Depletion of PAM sequences in-vitro was followed by Maxwell et al., Methods (2018). Briefly, linear DNA expressing the OMNI-50 nuclease and an sgRNA under a T7 promoter were added to a TXTL mix (Arbor Bioscience) together with a linear construct expressing T7 polymerase. RNA expression and protein translation by the TXTL mix result in the formation of the RNP complex. Since linear DNA was used, Chi6 sequences, a RecBCD inhibitor, were added to protect the DNA from degradation. The sgRNA spacer is designed to target a library of plasmids containing the targeting protospacer (pbPOS T2 library, Table 4) flanked by an 8N randomized set of potential PAM sequences. Depletion of PAM sequences from the library was measured by high-throughput sequencing upon using PCR to add the necessary adapters and indices to both the cleaved library and to a control library expressing a non-targeting gRNA (T1). Following deep sequencing, the in-vitro activity was confirmed by the fraction of the depleted sequences having the same PAM sequence relative to their occurrence in the control by the OMNI nuclease indicating functional DNA cleavage by an in-vitro system (FIG. 2, Table 3). OMNI-50 was tested with two sgRNA versions (V1 and V2). In both cases, a clear PAM of NGG was deduced from the analysis (FIG. 2). Some activity was also observed with NAG and NGA PAM sequences.

### PAM Library in Mammalian System

While a PAM sequence preference is considered as an inherent property of the nuclease, it may be affected, to some extent, by the cellular environment, genomic composition, and genome size. Since the human cellular environment is significantly different from the bacterial environment with respect to each of those properties, a “fine tuning” step has been introduced to address potential differences in PAM preferences in the human cellular context. To this end, a PAM library was constructed in a human cell line. In this assay, The PAM library was introduced to the cells using a viral vector (see Table 4) as a constant target sequence followed by a stretch of 6N. Upon introduction of OMNI-50 and an sgRNA targeting the library constant target site, NGS analysis was used to identify the edited sequences and the PAM associated with them. The enriched edited sequences were then used to define the PAM consensus. This methodology is applied to determine the optimized PAM requirements of the OMNI-50 nuclease in mammalian cells (Table 3, “Mammalian refinements”). The OMNI-50 PAM was found to be identical to the one found in the in-vitro TXTL.

### Expression of OMNI-50 Nuclease Coded by an Optimized DNA Sequence in Mammalian Cells

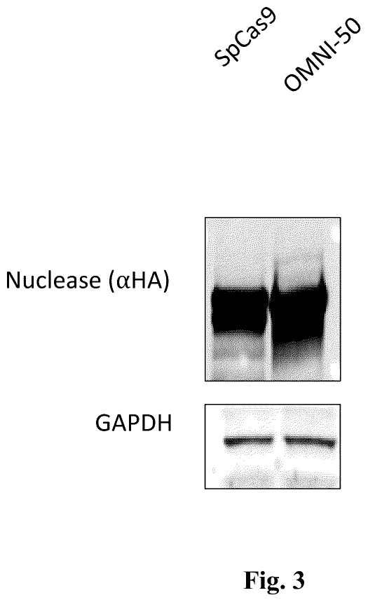

First, expression of each of the optimized DNA sequences encoding OMNI-50 in mammalian cells was validated. To this end, an expression vector coding for an HA-tagged OMNI-50 nuclease or Streptococcus Pyogenes Cas9 (SpCas9) linked to mCherry by a P2A peptide (pmOMNI, Table 4) was introduced into Hek293T cells using the Jet-optimus™ transfection reagent (polyplus-transfection). The P2A peptide is a self-cleaving peptide which can induce the cleaving of the recombinant protein in a cell such that the OMNI nuclease and the mCherry are separated upon expression. The mCherry serves as indicator for transcription efficiency of the OMNI from expression vector. Expression of OMNI-50 protein was confirmed by a western blot assay using an anti-HA antibody (FIG. 3).

### Activity in Human Cells on Endogenous Genomic Targets

OMNI-50 was also assayed for its ability to promote editing on specific genomic locations in human cells. To this end, an OMNI-P2A-mCherry expression vector (pmOMNI, Table 4) was transfected into HeLa cells together with an sgRNA designed to target a specific location in the human genome (pmGuide, Table 4). At 72 h, cells were harvested. Half of the cells were used for quantification of transfection efficiency by FACS using mCherry fluorescence as a marker. The other half of the cells were lysed, and their genomic DNA was used to PCR amplify the corresponding putative genomic targets. Amplicons were subjected to NGS and the resulting sequences were used calculate the percentage of editing events in each target site. Short insertions or deletions (indels) around the cut site are the typical outcome of repair of DNA ends following nuclease-induced DNA cleavage. The calculation of percent editing was deduced from the fraction of indel-containing sequences within each amplicon. All editing values were normalized to the transfection and translation efficacy obtained for each experiment and deduced from the percentage of mCherry expressing cells. The normalized values represent the effective editing levels within the population of cells that expressed the nuclease.

Genomic activity of OMNI-50 was assessed using a panel of eleven unique sgRNAs each designed to target a different genomic location. The results of these experiments are summarized in Table 6. As can be seen in the table (column 6, “% editing”), OMNI-50 exhibits high and significant editing levels compared to the negative control (column 9, “% editing in neg control”) in all target sites tested. OMNI-50 exhibits high and significant editing levels in 11/11 sites tested.

### Intrinsic Fidelity in Human Cells

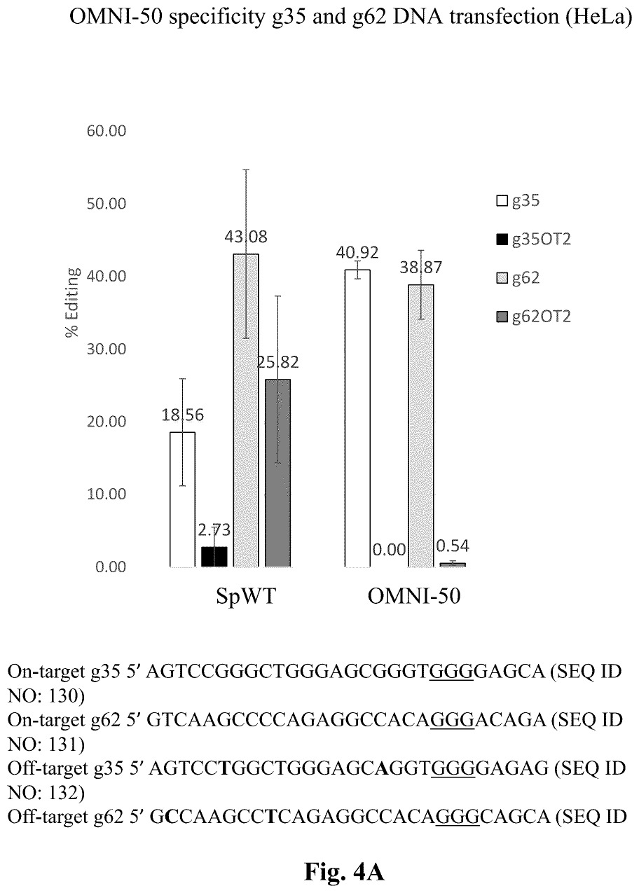

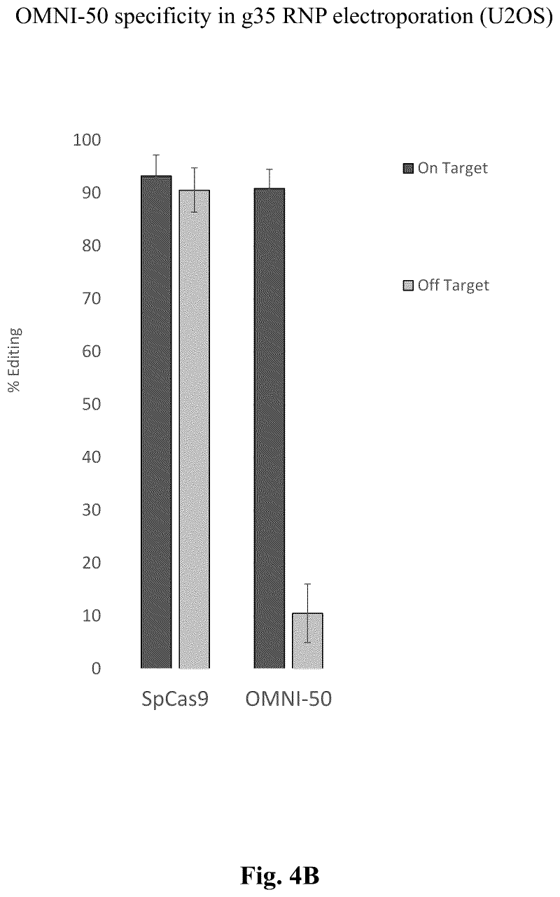

The intrinsic fidelity of a nuclease is a measure of its cleavage specificity. A high-fidelity nuclease is a nuclease that promotes cleavage on an intended target (“on-target”) with minimal or no cleavage of an unintended target (“off-target”). For CRISPR nucleases the target is acquired based on sequence complementarity to the spacer element of the guide RNA. Off-targeting results from similarity between the spacer sequence and an unintended target. The intrinsic fidelity of OMNI-50 at the genomic level in human cells was measured by conducting an activity assay as described in the section above, following PCR amplification, NGS, and indel analysis for both the on-target region and a pre-validated off-target region. A measurement of intrinsic fidelity for OMNI-50 is provided in FIG. 4A. In this example, OMNI-50 fidelity was measured using two guide RNAs independently, in each case a side by side measurement of SpCas9 is provided for reference. The first site was targeted using the ELANE g35 gRNA (Table 6) which has a defined on-target site upstream to the ELANE gene on chr19 and an off-target site on chr15. As can be seen in FIG. 4A, the on/off target editing efficiency ratio obtained by OMNI-50 was 41:0 while SpCas9 on/off ratio is 6.8:1 (40.9%/0%; 18.6%/2.7%, respectively). The second site was targeted by ELANE g62 gRNA (Table 6). This gRNA spacer sequence has a defined on-target site at the ELANE gene on chr19 and an off-target site on chr1. In this case, the on/off ratio obtained by OMNI-50 was 72:1 compared to 1.7:1 ratio obtained by SpCas9 (38.9%/0.6%; 43.1%/25.8%, respectively). These results demonstrate that OMNI-50 has a significantly higher intrinsic fidelity in comparison to SpCas9 using these specific gRNAs. Intrinsic fidelity was later tested in a second system by RNP electroporation into a U2OS cell line (FIG. 4B). For ELANE g35 the on/off target editing efficiency ratio obtained by OMNI-50 was 9:1 while the SpCas9 on/off ratio is 1:1 (91%/10%; 93%/91%, respectively). In two separate systems OMNI-50 fidelity was superior to SpCas9.

### Evaluating Off-Target Using a Guide-Seq Unbiased Analysis Method

To further evaluate the specificity of OMNI-50, the number of off-targets were tested across several sites using guide-seq. The off-targets count for SpCas9 varied across sites from several to hundreds, while the OMNI-50 off-targets count was lower than twenty in all sites tested. Comparing the number of off-targets found for sites having greater than 10 reads using either SpCas9 or OMNI-50 indicates the high specificity of OMNI-50. In five out of six sites tested, the number of SpCas9 off-targets was considerably higher compared to OMNI-50 (double to twenty-fold), while in only one of six sites the off-targets count is comparable between the two nucleases (Table 9).

Purification of OMNI-50 protein

The OMNI-50 open reading frame was cloned into bacterial expression plasmids (T7-NLS-OMNI-NLS-HA-His-tag, pET9a, Table 4) and expressed in C43 cells (Lucigen). Cells were grown in Terrific Broth to mid-log phase and the temperature was then lowered to 18° C. Expression was induced at 0.6 OD with 1 mM IPTG for 16-20 h before harvesting and freezing cells at −80° C. Cell paste was resuspended in lysis buffer (50 mM NaH₂PO₄, 300 mM NaCl, 10 mM imidazole pH8.0, 1 mM TCEP) supplemented with EDTA-free complete protease inhibitor cocktail set III (Calbiochem). Cells were lysed using sonication and cleared lysate was incubated with Ni-NTA resin. The resin was loaded onto a gravity column, washed with wash buffer (50 mM NaH₂PO₄, 300 mM NaCl, 50 mM imidazole pH8.0, 1 mM TCEP), and OMNI-50 protein was eluted with wash buffer supplemented with 100-500 mM imidazole. Fractions containing OMNI-50 protein were pooled, concentrated, loaded onto a centricone (Amicon Ultra 15 ml 100 K, Merck), and buffer exchanged to GF buffer (50 mM Tris-HCl pH 7.5, 500 mM NaCl, 10% glycerol, 0.4M Arginine). The concentrated OMNI-50 protein was further purified by SEC on HiLoad 16/600 Superdex 200 pg-SEC, AKTA Pure (GE Healthcare Life Sciences) with a 50 mM Tris-HCl pH 7.5, 500 mM NaCl, 10% glycerol, 0.4M Arginine. Fractions containing OMNI-50 protein were pooled, concentrated, and loaded onto a centricone (Amicon Ultra 15 ml 100 K, Merck) with a final storage buffer of 10 mM Tris-HCl, pH 7.5, 150 mM NaCl, 10% glycerol and 1 mM TCEP. Purified OMNI-50 protein was concentrated to 10 mg/ml stocks, flash-frozen in liquid nitrogen, and stored at −80° C.

### Guide Optimization by RNP Activity Assay

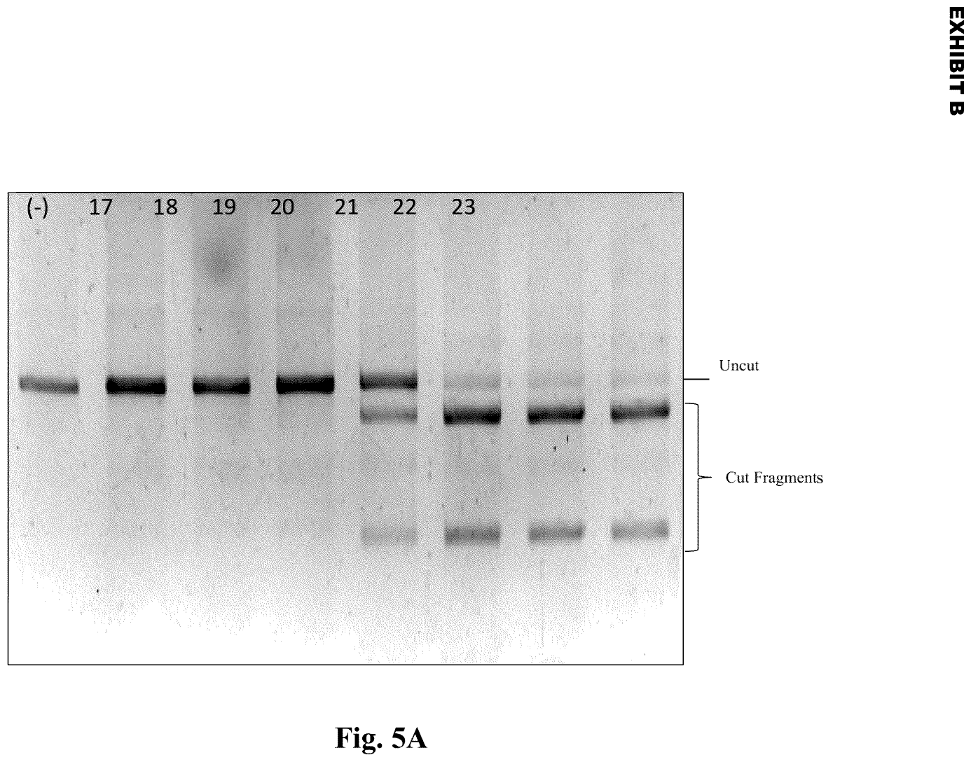

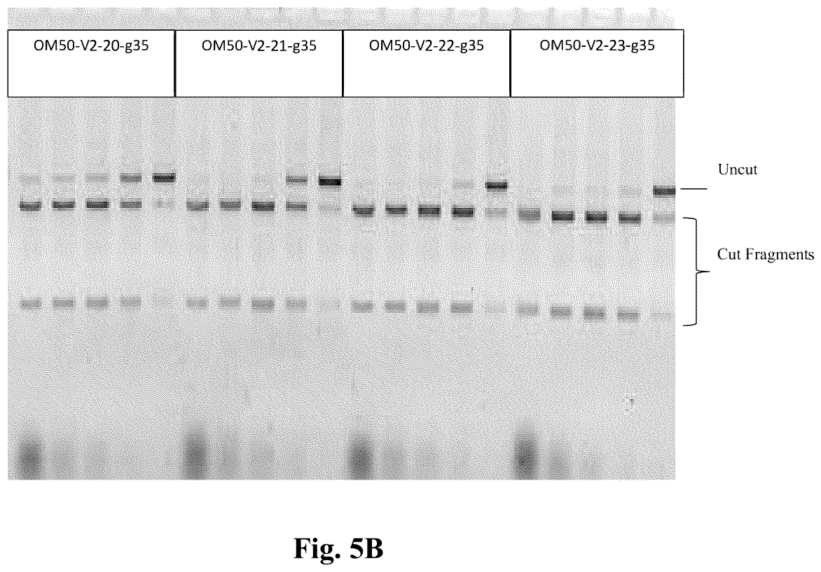

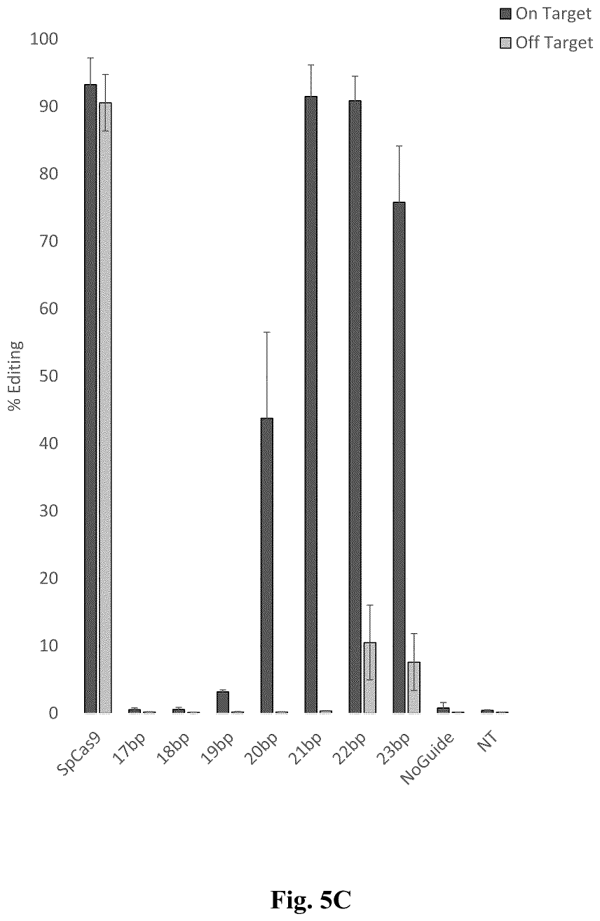

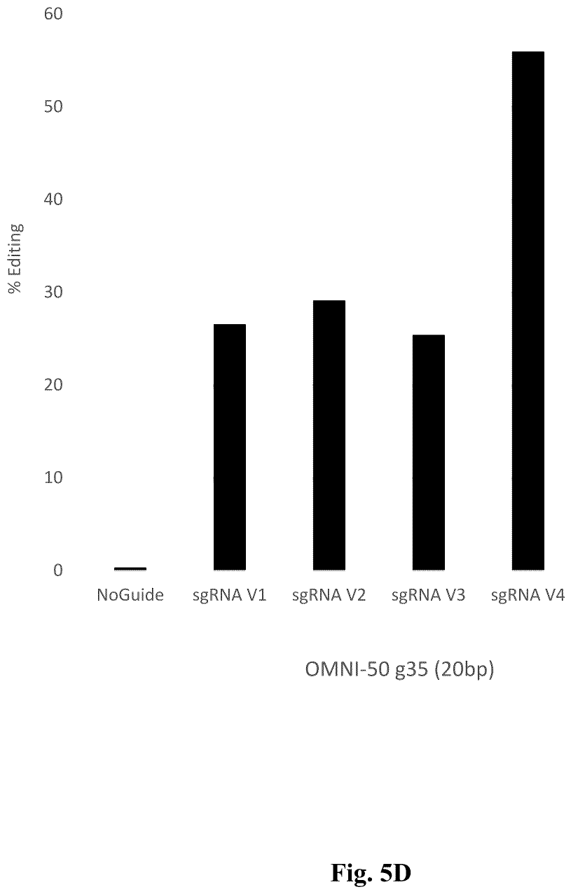

Synthetic sgRNAs of OMNI-50 were synthesized with three 2′-O-methyl 3′-phosphorothioate at the 3′ and 5′ ends (Agilent). An activity assay of OMNI-50 RNPs with different spacer lengths (17-23 nts) of guide 35 is described herein (Table 5, FIG. 5A). Briefly, 4 pmol of OMNI-50 nuclease was mixed with 6 pmol of synthetic guide. After 10 minutes of incubation at room temperature, the RNP complexes were reacted with 100 ng of on-target template. Only spacer greater than or equal to 22 nts show near full cleavage of the on-target template. When decreasing amounts of RNPs (4, 2, 1.2, 0.6 and 0.2 pmol) having spacer lengths 20-23 nts were reacted with 100 ng of DNA target template (FIG. 5B). Spacer at lengths greater than or equal to 22 nt show better cleavage activity even at lower RNP concentrations.

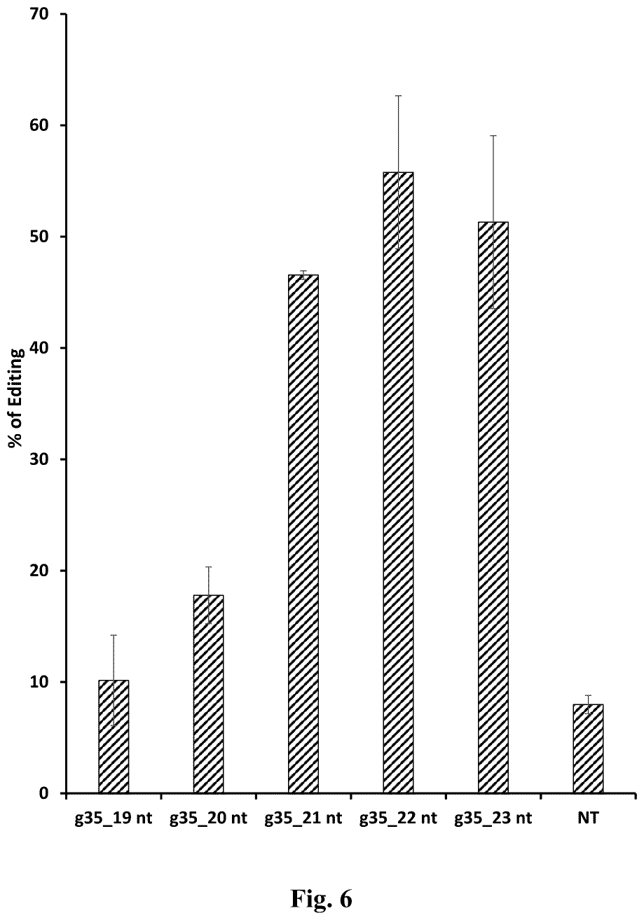

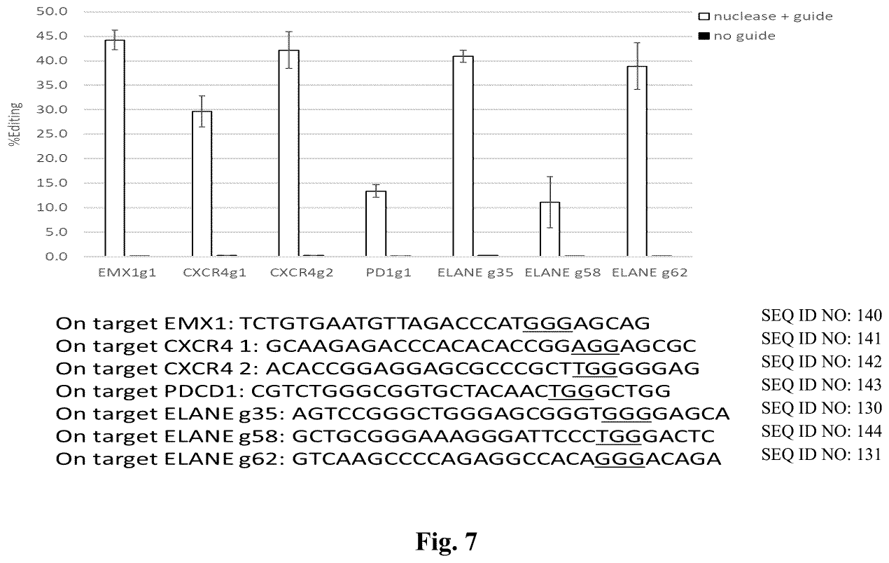

Spacer length optimization was also performed in a mammalian cell context. RNPs were assembled by mixing 100 uM nuclease with 120 uM of synthetic guide with different spacer lengths (17-23 nt, Table 5) and 100 uM Cas9 electroporation enhancer (IDT). After 10 minutes of incubation at room temperature, the RNP complexes were mixed with 200,000 pre-washed U205, iPSC, or HSC cells and electroporated using Lonza SE or P3 Cell Line 4D-Nucleofector™ X Kit with the DN100 or CA137 program, respectively, according to the manufacture's protocol. At 72 h cells were lysed and their genomic DNA was used in a PCR reaction to amplify the corresponding putative genomic targets. Amplicons were subjected to NGS and the resulting sequences were then used to calculate the percentage of editing events. As can be seen in FIG. 5C, FIG. 6, and Table 10, spacers of 17-19 nts show a low editing level, 20 nt spacers show a medium editing level, and spacers of 21-23 nts show the highest editing level.

Using the U2OS cell line, different tracer RNA sequence variations were tested (Table 2). The different sgRNA versions were tested with a 20 nt spacer. As can be seen in FIG. 5D, RNP assembly using either sgRNA V1, V2, or V3 results in a similar editing level. However, RNP assembly using sgRNA V4 results in a significantly higher editing level.

Comparing results obtained in HSCs using 21 nt and 22 nt spacers across five genomic sites suggests that a 22 nt spacer is slightly preferred for efficient editing (FIG. 6 and table 10).

### Activity of OMNI-50 as an RNP

Activity of OMNI-50 protein as RNP in mammalian cells was first tested in the U2OS cell line, and later tested in three primary cell systems: iPSCs, HSCs, and T cells. As can be seen in Table 7, editing was observed in all systems.

OMNI-50 was tested for editing activity in T-cells on two genes (Appendix Table 7). OMNI-50 was tested with 34 guides targeting TRAC and 26 guides targeting B2M. 64% (22/34) of the tested TRAC guides were found to be active, with editing levels ranging between 5% to 84%. Similarly, 57% of the B2M guides were active, with editing levels ranging between 5% and 61%. These results are summarized in Appendix Table 7.

High editing was observed in both TRAC and B2M genes in a repertoire of 19 guides each. Considering the potential for multiplexing and further optimization, full knock-out of both genes by OMNI-50 is possible with the appropriate strategy.

In U2OS cells, iPSCs and HSCs, guides targeting the ELANE gene were tested with OMNI-50. All five guides tested showed editing above 22% in both U2OS cells and HSCs. In iPSCs only ELANE g35 was tested with editing level of 53%. This result is lower compared with the results obtained with other systems.

### Multiplexing

OMNI-50 was also tested for multiplex editing by mixing two RNP populations and electroporating the mix into primary T cells. gRNA #32 was used for TRAC, and gRNA #15 was used for B2M (spacer sequences are listed in Table 8). At 72 h cells were harvested and tested for editing by NGS. The TRAC gene measured 50% editing, and the B2M gene measured 25% editing. These results were similar to editing levels with a single RNP that was performed side-by-side to the multiplex test (Table 8).

TABLE 1
OMNI-50 nuclease sequences

| | | | SEQ ID NO of DNA |
|---|---|---|---|
| | SEQ ID NO | SEQ ID NO of | sequence codon |
| | of OMNI-50 | DNA sequence | optimized for |
| Source | Amino Acid | encoding | encoding OMNI-50 |
| Organism | Sequence | OMNI-50 | in human cells |
| Ezakiella | 3 | 11 | 12, 13 |
| peruensis strain | | | |
| M6.X2 | | | |

Table 1. OMNI-50 nuclease sequences: Table 1 lists the organism from which the OMNI-50 nuclease was identified, its protein sequence, its DNA sequence, and its human optimized DNA sequence(s).

TABLE 2
OMNI-50 guide sequences

| | | |
|---|---|---|
| Minimal | crRNA | GUUUGAGAG |
| crRNA:tracrRNA | (Repeat) | |
| duplex | tracrRNA | CGAGUUCAAAU (SEQ ID NO: 149) |
| | (Antirepeat) | |
| crRNA:tracrRNA | crRNA | GUUUGAGAGUUAUG (SEQ ID NO: 37) |
| duplex V1 | (Repeat) | |
| | tracrRNA | CAUGACGAGUUCAAAU (SEQ ID NO: 38) |
| | (Antirepeat) | |
| crRNA:tracrRNA | crRNA | GUUUGAGAGUUAUGUAA (SEQ ID NO: 39) |
| duplex V2 | (Repeat) | |
| | tracrRNA | UUACAUGACGAGUUCAAAU (SEQ ID NO: 40) |
| | (Antirepeat) | |
| TracrRNA | TracrRNA | AAAAAUUUAUUCAAACC (SEQ ID NO: 150) |
| sequences | Portion 1 | |
| | TracrRNA | GCCUAUUUAUAGGC (SEQ ID NO: 42) |
| | Portion 2 | |
| | TracrRNA | CGCAGAUGUUCUGC (SEQ ID NO: 151) |
| | Portion 3 | |
| | TracrRNA | AUUAUGCUUGCUAUUGCAAGCUUUUUU (SEQ ID NO: 152) |
| | Portion 4 | |
| | Full | CAUGACGAGUUCAAAUAAAAAUUUAUUCAAACCGCCUAUUUAUA |
| | tracrRNA | GGCCGCAGAUGUUCUGCAUUAUGCUUGCUAUUGCAAGCUUUUUU |
| | V1 | (SEQ ID NO: 153) |
| | Full | UUACAUGACGAGUUCAAAUAAAAAUUUAUUCAAACCGCCUAUUU |
| | tracrRNA | AUAGGCCGCAGAUGUUCUGCAUUAUGCUUGCUAUUGCAAGCUUU |
| | V2 | UUU (SEQ ID NO: 154) |
| sgRNA Versions | sgRNA V1 | GUUUGAGAGUUAUGgaaaCAUGACGAGUUCAAAUAAAAAUUUAUU |
| | | CAAACCGCCUAUUUAUAGGCCGCAGAUGUUCUGCAUUAUGCUUG |
| | | CUAUUGCAAGCUUUUUU (SEQ ID NO: 44) |
| | sgRNA V2 | GUUUGAGAGUUAUGUAAgaaaUUACAUGACGAGUUCAAAUAAAAA |
| | | UUUAUUCAAACCGCCUAUUUAUAGGCCGCAGAUGUUCUGCAUUA |
| | | UGCUUGCUAUUGCAAGCUUUUUU (SEQ ID NO: 45) |
| Other sgRNA | sgRNA V3 | GUUUGAGAGUUAUGUgaaaACAUGACGAGUUCAAAUAAAAAUUUA |
| Optimizations | | UUCAAACCGCCUAUUUAUAGGCCGCAGAUGUUCUGCAUUAUGCU |
| | | UGCUAUUGCAAGCUUUUUU (SEQ ID NO: 87) |
| | sgRNA V4 | GUUUGAGAGUUAUGUAgaaaUACAUGACGAGUUCAAAUAAAAAUU |
| | | UAUUCAAACCGCCUAUUUAUAGGCCGCAGAUGUUCUGCAUUAUG |
| | | CUUGCUAUUGCAAGCUUUUUU (SEQ ID NO: 88) |

TABLE 3
OMNI-50 PAM sequences

| | | |
|---|---|---|
| TXTL Depletion | PAM General | NGG |
| | PAM Specific | NGG |
| | Activity (1-Depletion score)* | 0.98 |
| | sgRNA | V1, V2 |
| Mammalian | PAM Mammlian | NGG |
| refinements | | |

*Depletion score-Average of the ratios from two most depleted sites

TABLE 4
Plasmids and Constructs

| Plasmid | Purpose | Elements | Example |
|---|---|---|---|
| pbNNC-2 | Expressing OMNI | T7 promoter HA Tag- | pbNNC2 OMNI-50 |
| | polypeptide in the bacterial | Linker-OMNI ORF | |
| | system | (Human optimized)-T7 | |
| | | terminator | |
| pbGuide | Expressing OMNI sgRNA in | J23119 promoter-T1/T2 | pbGuide OMNI-50 T2 |
| T1/T2 | the bacterial system | spacer sgRNA scaffold- | sgRNA V2 |
| | | rrnB T1 terminator | |
| pbPOS T2 | Bacterial/TXTL depletion | T2 protospacer-8N PAM | pbPOS T2 library |
| library | assay | library-chloramphenicol | |
| | | acetyltransferase | |
| pET9a | Expression and purification | T7 promoter-SV40 NLS- | pET9a OMNI-50- |
| | of OMNI proteins | OMNI ORF (human | HisTag |
| | | optimized)-HA-SV40 | |
| | | NLS-8 His-tag-T7 | |
| | | terminator | |
| pmOMNI | Expressing OMNI | CMV promoter-Kozak- | pmOMNI OMNI-50 |
| | polypeptide in the | SV40 NLS-OMNI ORF | |
| | mammalian system | (human optimized)-HA- | |
| | | SV40 NLS-P2A- | |
| | | mCherry-Bgh poly(A) | |
| | | signal | |
| pmGuide | Expressing OMNI sgRNA in | U6 promoter-Endogenic | pmGuide OMNI-50 |
| Endogenic | the mammalian system | spacer sgRNA scaffold | CXCR4 sgRNA V3 |
| site | | | |
| pPM3L3.1 | Viral vector for PAM library | LTR-HIV-1 Ψ-CMV | pPM3L3.1 |
| | in mammalian cells | promoter-T2-PAM | |
| | | library (6N)-GFP-SV40 | |
| | | promoter-blastocydin S | |
| | | deaminase-LTR | |

TABLE 4
Appendix-Details of construct elements

| Element | Protein Sequence | DNA sequence |
|---|---|---|
| HA Tag | SEQ ID NO: 63 | SEQ ID NO: 64 |
| NLS | SEQ ID NO: 65 | SEQ ID NO: 66 |
| P2A | SEQ ID NO: 85 | SEQ ID NO: 86 |
| mCherry | SEQ ID NO: 67 | SEQ ID NO: 68 |

TABLE 5
Synthetic sgRNA (spacer and scaffold)

| | O50_ELANE_ | O50_ELANE_ | O50_ELANE_V2_ | O50_ELANE_V2_ | O50_ELANE_V2_ |
|---|---|---|---|---|---|
| Name | V2_g35_23 | V2_g35_22 | g35_21 | g35_20 | g35_19 |
| Spacer | UgcAGUCC | gcAGUCCG | cAGUCCGG | AGUCCGG | GUCCGGG |
| | GGGCUGG | GGCUGGG | GCUGGGA | GCUGGGA | CUGGGAG |
| | GAGCGGG | AGCGGGU | GCGGGU | GCGGGU | CGGGU |
| | U (SEQ ID | (SEQ ID NO: | (SEQ ID NO: | (SEQ ID NO: | (SEQ ID NO: |
| | NO: 112) | 116) | 118) | 120) | 122) |
| Scaffold | gUUUGAG | gUUUGAG | gUUUGAG | gUUUGAG | gUUUGAG |
| | AGUUAUG | AGUUAUG | AGUUAUG | AGUUAUG | AGUUAUG |
| | UAAgaaaU | UAAgaaaU | UAAgaaaU | UAAgaaaU | UAAgaaaU |
| | UACAUGA | UACAUGA | UACAUGA | UACAUGA | UACAUGA |
| | CGAGUUC | CGAGUUC | CGAGUUC | CGAGUUC | CGAGUUC |
| | AAAUAAA | AAAUAAA | AAAUAAA | AAAUAAA | AAAUAAA |
| | AAUUUAU | AAUUUAU | AAUUUAU | AAUUUAU | AAUUUAU |
| | UCAAACC | UCAAACC | UCAAACC | UCAAACC | UCAAACC |
| | GCCUAUU | GCCUAUU | GCCUAUU | GCCUAUU | GCCUAUU |
| | UAUAGGC | UAUAGGC | UAUAGGC | UAUAGGC | UAUAGGC |
| | CGCAGAU | CGCAGAU | CGCAGAU | CGCAGAU | CGCAGAU |
| | GUUCUGC | GUUCUGC | GUUCUGC | GUUCUGC | GUUCUGC |
| | AUUAUGC | AUUAUGC | AUUAUGC | AUUAUGC | AUUAUGC |
| | UUGCUAU | UUGCUAU | UUGCUAU | UUGCUAU | UUGCUAU |
| | UGCAAGC | UGCAAGC | UGCAAGC | UGCAAGC | UGCAAGC |
| | UUUUUU | UUUUUU | UUUUUU | UUUUUU | UUUUUU |
| | (SEQ ID NO: | (SEQ ID NO: | (SEQ ID NO: | (SEQ ID NO: | (SEQ ID NO: |
| | 45) | 45) | 45) | 45) | 45) |
| Version | V2 | V2 | V2 | V2 | V2 |
| Full sgRNA | UgcAGUCC | gcAGUCCG | cAGUCCGG | AGUCCGG | GUCCGGG |
| sequence | GGGCUGG | GGCUGGG | GCUGGGA | GCUGGGA | CUGGGAG |
| | GAGCGGG | AGCGGGU | GCGGGUg | GCGGGUg | CGGGUgU |
| | UgUUUGA | gUUUGAG | UUUGAGA | UUUGAGA | UUGAGAG |
| | GAGUUAU | AGUUAUG | GUUAUGU | GUUAUGU | UUAUGUA |
| | GUAAgaaa | UAAgaaaU | AAgaaaUU | AAgaaaUU | AgaaaUUAC |
| | UUACAUG | UACAUGA | ACAUGAC | ACAUGAC | AUGACGA |
| | ACGAGUU | CGAGUUC | GAGUUCA | GAGUUCA | GUUCAAA |
| | CAAAUAA | AAAUAAA | AAUAAAA | AAUAAAA | UAAAAAU |
| | AAAUUUA | AAUUUAU | AUUUAUU | AUUUAUU | UUAUUCA |
| | UUCAAAC | UCAAACC | CAAACCG | CAAACCG | AACCGCC |
| | CGCCUAU | GCCUAUU | CCUAUUU | CCUAUUU | UAUUUAU |
| | UUAUAGG | UAUAGGC | AUAGGCC | AUAGGCC | AGGCCGC |
| | CCGCAGA | CGCAGAU | GCAGAUG | GCAGAUG | AGAUGUU |
| | UGUUCUG | GUUCUGC | UUCUGCA | UUCUGCA | CUGCAUU |
| | CAUUAUG | AUUAUGC | UUAUGCU | UUAUGCU | AUGCUUG |
| | CUUGCUA | UUGCUAU | UGCUAUU | UGCUAUU | CUAUUGC |
| | UUGCAAG | UGCAAGC | GCAAGCU | GCAAGCU | AAGCUUU |
| | CUUUUUU | UUUUUU | UUUUU | UUUUU | UUU (SEQ |
| | (SEQ ID NO: | (SEQ ID NO: | (SEQ ID NO: | (SEQ ID NO: | ID NO: 123) |
| | 113) | 117) | 119) | 121) | |
| Protospacer | CTGTTGCT | CTGTTGCT | CTGTTGCT | CTGTTGCT | CTGTTGCT |
| (with PAM | GCAGTCC | GCAGTCC | GCAGTCC | GCAGTCC | GCAGTCC |
| bolded) - | GGGCTGG | GGGCTGG | GGGCTGG | GGGCTGG | GGGCTGG |
| On target | GAGCGGG | GAGCGGG | GAGCGGG | GAGCGGG | GAGCGGG |
| | TGGGGAG | TGGGGAG | TGGGGAG | TGGGGAG | TGGGGAG |
| | CAGAGGG | CAGAGGG | CAGAGGG | CAGAGGG | CAGAGGG |
| | (SEQ ID NO: | (SEQ ID NO: | (SEQ ID NO: | (SEQ ID NO: | (SEQ ID NO: |
| | 114) | 114) | 114) | 114) | 114) |
| Protospacer | GTTAAGAg | GTTAAGAg | GTTAAGAg | GTTAAGAg | sGTTAAGAg |
| (with PAM | aCAGTCCa | aCAGTCCa | aCAGTCCa | aCAGTCCa | aCAGTCCa |
| bolded) - | GGCTGGG | GGCTGGG | GGCTGGG | GGCTGGG | GGCTGGG |
| Off target | AGCaGGT | AGCaGGT | AGCaGGT | AGCaGGT | AGCaGGT |
| | GGGGAGA | GGGGAGA | GGGGAGA | GGGGAGA | GGGGAGA |
| | GGAGGG | GGAGGG | GGAGGG | GGAGGG | GGAGGG |
| | (SEQ ID NO: | (SEQ ID NO: | (SEQ ID NO: | (SEQ ID NO: | (SEQ ID NO: |
| | 115) | 115) | 115) | 115) | 115) |

| | O50_ELANE_V2_ | O50_ELANE_V2_ | O50_ELANE_V3_ | |
|---|---|---|---|---|
| Name | g35_18 | g35_17 | g35_20 | O50_ELANE_V4_g35_20 |
| Spacer | UCCGGGCUG | CCGGGCUGG | AGUCCGGGC | AGUCCGGGC |
| | GGAGCGGGU | GAGCGGGU | UGGGAGCGG | UGGGAGCGG |
| | (SEQ ID NO: | (SEQ ID NO: | GU (SEQ ID | GU (SEQ ID |
| | 124) | 126) | NO: 120) | NO: 120) |
| Scaffold | gUUUGAGAG | gUUUGAGAG | gUUUGAGAG | gUUUGAGAG |
| | UUAUGUAAga | UUAUGUAAga | UUAUGUgaaaA | UUAUGUAgaaa |
| | aaUUACAUGA | aaUUACAUGA | CAUGACGAG | UACAUGACG |
| | CGAGUUCAA | CGAGUUCAA | UUCAAAUAA | AGUUCAAAU |
| | AUAAAAAUU | AUAAAAAUU | AAAUUUAUU | AAAAAUUUA |
| | UAUUCAAAC | UAUUCAAAC | CAAACCGCCU | UUCAAACCG |
| | CGCCUAUUU | CGCCUAUUU | AUUUAUAGG | CCUAUUUAU |
| | AUAGGCCGC | AUAGGCCGC | CCGCAGAUG | AGGCCGCAG |
| | AGAUGUUCU | AGAUGUUCU | UUCUGCAUU | AUGUUCUGC |
| | GCAUUAUGC | GCAUUAUGC | AUGCUUGCU | AUUAUGCUU |
| | UUGCUAUUG | UUGCUAUUG | AUUGCAAGC | GCUAUUGCA |
| | CAAGCUUUU | CAAGCUUUU | UUUUUU (SEQ | AGCUUUUUU |
| | UU (SEQ ID | UU (SEQ ID | ID NO: 87) | (SEQ ID NO: 88) |
| | NO: 45) | NO: 45) | | |
| Version | V2 | V2 | V3 | V4 |
| Full sgRNA | UCCGGGCUG | CCGGGCUGG | AGUCCGGGC | AGUCCGGGC |
| sequence | GGAGCGGGUg | GAGCGGGUgU | UGGGAGCGG | UGGGAGCGG |
| | UUUGAGAGU | UUGAGAGUU | GUgUUUGAG | GUgUUUGAG |
| | UAUGUAAgaaa | AUGUAAgaaaU | AGUUAUGUga | AGUUAUGUA |
| | UUACAUGAC | UACAUGACG | aaACAUGACG | gaaaUACAUGA |
| | GAGUUCAAA | AGUUCAAAU | AGUUCAAAU | CGAGUUCAA |
| | UAAAAAUUU | AAAAAUUUA | AAAAAUUUA | AUAAAAAUU |
| | AUUCAAACC | UUCAAACCG | UUCAAACCG | UAUUCAAAC |
| | GCCUAUUUA | CCUAUUUAU | CCUAUUUAU | CGCCUAUUU |
| | UAGGCCGCA | AGGCCGCAG | AGGCCGCAG | AUAGGCCGC |
| | GAUGUUCUG | AUGUUCUGC | AUGUUCUGC | AGAUGUUCU |
| | CAUUAUGCU | AUUAUGCUU | AUUAUGCUU | GCAUUAUGC |
| | UGCUAUUGC | GCUAUUGCA | GCUAUUGCA | UUGCUAUUG |
| | AAGCUUUUU | AGCUTJUTJUU | AGCUUUUUU | CAAGCUUUU |
| | U (SEQ ID NO: | (SEQ ID NO: | (SEQ ID NO: | UU (SEQ ID |
| | 125) | 127) | 128) | NO: 129) |
| Protospacer | CTGTTGCTGC | CTGTTGCTGC | CTGTTGCTGC | CTGTTGCTGC |
| (with PAM | AGTCCGGGCT | AGTCCGGGCT | AGTCCGGGCT | AGTCCGGGCT |
| bolded) - | GGGAGCGGG | GGGAGCGGG | GGGAGCGGG | GGGAGCGGG |
| On target | TGGGGAGCA | TGGGGAGCA | TGGGGAGCA | TGGGGAGCA |
| | GAGGG (SEQ | GAGGG (SEQ | GAGGG (SEQ | GAGGG (SEQ |
| | ID NO: 114) | ID NO: 114) | ID NO: 114) | ID NO: 114) |
| Protospacer | GTTAAGAgaC | GTTAAGAgaC | GTTAAGAgaC | GTTAAGAgaC |
| (with PAM | AGTCCaGGCT | AGTCCaGGCT | AGTCCaGGCT | AGTCCaGGCT |
| bolded) - | GGGAGCaGGT | GGGAGCaGGT | GGGAGCaGGT | GGGAGCaGGT |
| Off target | GGGGAGAGG | GGGGAGAGG | GGGGAGAGG | GGGGAGAGG |
| | AGGG (SEQ ID | AGGG (SEQ ID | AGGG (SEQ ID | AGGG (SEQ ID |
| | NO: 115) | NO: 115) | NO: 115) | NO: 115) |

TABLE 6
Activity of OMNI-50 in human cells on endogenous genomic targets

| | Corre- | | 3′ (PAM | | | | % | % trans- | Norm. % |
|---|---|---|---|---|---|---|---|---|---|
| | sponding | | containing) | | % | Norm. | editing | fection | editing |
| Genomic | Spacer | Spacer | genomic seq | % | trans- | % | in neg | in neg | in neg |
| site | name | sequence | (PAM Bolded) | indels | fection | editing | control | control | control |
| EMX1 | EMX1g1_ | UCUGUG | GGGAG | 44.18- | | | 0.02 | | |
| site 2 | OMNI50 | AAUGUU | CAG | 25.72 | | | | | |
| | | AGACCC | | | | | | | |
| | | AU (SEQ | | | | | | | |
| | | ID NO: 97) | | | | | | | |
| EMX1 | EMX1g2_ | CCAUGG | AGGGG | 55.81 | | | | | |
| site 3 | OMNI50 | GAGCAG | ACC | | | | | | |
| | | CUGGUC | | | | | | | |
| | | AG (SEQ | | | | | | | |
| | | ID NO: 98) | | | | | | | |
| CXCR4 | CXCR4g1_ | GCAAGA | AGGAG | 29.58- | | | 0.18 | | |
| site 3 | OMNI50 | GACCCA | CGC | 32.14 | | | | | |
| | | CACACC | | | | | | | |
| | | GG (SEQ | | | | | | | |
| | | ID NO: 99) | | | | | | | |
| CXCR4 | CXCR4g2_ | ACACCG | TGGGG | 42.13- | | | 0.22 | | |
| site 4 | OMNI50 | GAGGAG | GAG | 49.85 | | | | | |
| | | CGCCCG | | | | | | | |
| | | CU (SEQ | | | | | | | |
| | | ID NO: | | | | | | | |
| | | 100) | | | | | | | |
| PDCD1 | PDCD1g1_ | CGUCUG | TGGGCT | 13.35- | | | 0.05 | | |
| site 4 | OMNI50 | GGCGGU | GG | 8.7 | | | | | |
| | | GCUACA | | | | | | | |
| | | AC (SEQ | | | | | | | |
| | | ID NO: | | | | | | | |
| | | 101) | | | | | | | |
| PDCD1 | PDCD1g2_ | CUACAA | AGGAT | 17.53 | | | | | |
| site 5 | OMNI50 | CUGGGC | GGT | | | | | | |
| | | UGGCGG | | | | | | | |
| | | CC (SEQ | | | | | | | |
| | | ID NO: 102) | | | | | | | |
| ELANE | ELANEg3 | AGUCCG | GGGGA | 40.92- | | | 0.24 | 5.95 | 3.982225429 |
| g35 | 5_OMNI50 | GGCUGG | GCA | 55.39 | | | | | |
| | | GAGCGG | | | | | | | |
| | | GU (SEQ | | | | | | | |
| | | ID NO: 103) | | | | | | | |
| ELANE | ELANEg5 | GCUGCG | TGGGA | 11.11 | 20.50 | 54.23 | 0.18 | 5.95 | 2.974553445 |
| g58 | 8_OMNI50 | GGAAAG | CTC | | | | | | |
| | | GGAUUC | | | | | | | |
| | | CC (SEQ | | | | | | | |
| | | ID NO: 104) | | | | | | | |
| ELANE | ELANEg3 | ACAGCG | GGGGG | 9.99 | | | | | |
| g38 | 8 OMNI50 | GGUGUA | ACG | | | | | | |
| | | GACUCC | | | | | | | |
| | | GA (SEQ | | | | | | | |
| | | ID NO: 105) | | | | | | | |
| ELANE | ELANEg3 | CAGCGG | GGGGA | 24.87 | | | | | |
| g39 | 9_OMNI50 | GUGUAG | CGT | | | | | | |
| | | ACUCCG | | | | | | | |
| | | AG | | | | | | | |
| | | (SEQ ID | | | | | | | |
| | | NO: 106) | | | | | | | |
| ELANE | ELANEg6 | GUCAAG | GGGAC | 38.87- | | | 0.12 | 5.95 | 2.002503126 |
| g62 | 2_OMNI50 | CCCCAG | AGA | 52.74 | | | | | |
| | | AGGCCA | | | | | | | |
| | | CA | | | | | | | |
| | | (SEQ ID | | | | | | | |
| | | NO: 107) | | | | | | | |

Table 6. Nuclease activity in endogenous context in mammalian cells: The OMNI-50 nuclease was expressed in mammalian cell system (HeLa) by DNA transfection together with an sgRNA expressing plasmid. Cell lysates were used for site specific genomic DNA amplification and NGS. The percentage of indels was measured and analyzed to determine editing level. Each sgRNA is composed of the tracrRNA (see Table 2) and the spacer detailed here. The 3′ genomic spacer sequence contains the PAM relevant for the OMNI-50 nuclease. Transfection efficiency (% transfection) was measured by flow cytometry quantification of mCherry signal, as described above. The transfection efficiency was used to normalize the editing level (% indels norm). All tests were performed in triplicate. OMNI nuclease only (i.e. no guide) transfected cells served as a negative control.

TABLE 7
OMNI-50 Activity as an RNP

| | Genomic | Corresponding | | % |
|---|---|---|---|---|
| System | site | spacer name | Spacer sequence | indels |
| Primary T | TRAC | gRNA 1 | TCTCTCAGCTGGTACACGGCA | 18% |
| cells | | | (SEQ ID NO: 156) | |
| | | gRNA 2 | GCGTCATGAGCAGATTAAACC | 81% |
| | | | (SEQ ID NO: 157) | |
| | | gRNA 3 | TCTCGACCAGCTTGACATCAC | 10% |
| | | | (SEQ ID NO: 158) | |
| | | gRNA 4 | TTAAACCCGGCCACTTTCAGG | 46% |
| | | | (SEQ ID NO: 159) | |
| | | gRNA 5 | CTGTGCTAGACATGAGGTCTA | 26% |
| | | | (SEQ ID NO: 160) | |
| | | gRNA 8 | ACTTCAAGAGCAACAGTGCTG | 3% |
| | | | (SEQ ID NO: 161) | |
| | | gRNA 9 | AAGAGCAACAGTGCTGTGGCC | 13% |
| | | | (SEQ ID NO: 162) | |
| | | gRNA 10 | GCTGGGGAAGAAGGTGTCTTC | 7% |
| | | | (SEQ ID NO: 163) | |
| | | gRNA 15 | ATAGGCAGACAGACTTGTCAC | 16% |
| | | | (SEQ ID NO: 164) | |
| | | gRNA 17 | TAGAGTCTCTCAGCTGGTACA | 23% |
| | | | (SEQ ID NO: 165) | |
| | | gRNA 18 | GTCTCTCAGCTGGTACACGGC | 5% |
| | | | (SEQ ID NO: 166) | |
| | | gRNA 19 | CAGCTGGTACACGGCAGGGTC | 11% |
| | | | (SEQ ID NO: 167) | |
| | | gRNA 20 | AGCTGGTACACGGCAGGGTCA | 13% |
| | | | (SEQ ID NO: 168) | |
| | | gRNA 21 | TACACGGCAGGGTCAGGGTTC | 19% |
| | | | (SEQ ID NO: 169) | |
| | | gRNA 23 | CTTTCAAAACCTGTCAGTGAT | 4% |
| | | | (SEQ ID NO: 170) | |
| | | gRNA 25 | TCCGAATCCTCCTCCTGAAAG | 21% |
| | | | (SEQ ID NO: 171) | |
| | | gRNA 26 | AATCCTCCTCCTGAAAGTGGC | 11% |
| | | | (SEQ ID NO: 172) | |
| | | gRNA 27 | ATCCTCCTCCTGAAAGTGGCC | 9% |
| | | | (SEQ ID NO: 173) | |
| | | gRNA 29 | CTGCTCATGACGCTGCGGCTG | 15% |
| | | | (SEQ ID NO: 174) | |
| | | gRNA 30 | AGATTAAACCCGGCCACTTTC | 24% |
| | | | (SEQ ID NO: 175) | |
| | | gRNA 31 | AACCCGGCCACTTTCAGGAGG | 29% |
| | | | (SEQ ID NO: 176) | |
| | | gRNA 32 | GCCACTTTCAGGAGGAGGATT | 29% |
| | | | (SEQ ID NO: 177) | |
| | B2M | gRNA 1 | TACTCTCTCTTTCTGGCCTGG | 5% |
| | | | (SEQ ID NO: 178) | |
| | | gRNA 2 | GCATACTCATCTTTTTCAGTG | 12% |
| | | | (SEQ ID NO: 179) | |
| | | gRNA 3 | CGCTACTCTCTCTTTCTGGCC | 17% |
| | | | (SEQ ID NO: 180) | |
| | | gRNA 4 | GCGCGAGCACAGCTAAGGCCA | 64% |
| | | | (SEQ ID NO: 181) | |
| | | gRNA 6 | GCTCGCGCTACTCTCTCTTTC | 9% |
| | | | (SEQ ID NO: 182) | |
| | | gRNA 7 | AGAGTAGCGCGAGCACAGCTA | 61% |
| | | | (SEQ ID NO: 183) | |
| | | gRNA 15 | TCACAGCCCAAGATAGTTAAG | 45% |
| | | | (SEQ ID NO: 184) | |
| | | gRNA 16 | CACAGCCCAAGATAGTTAAGT | 42% |
| | | | (SEQ ID NO: 185) | |
| | | gRNA 18 | GACAAAGTCACATGGTTCACA | 43% |
| | | | (SEQ ID NO: 186) | |
| | | gRNA 19 | AAGTCACATGGTTCACACGGC | 8% |
| | | | (SEQ ID NO: 187) | |
| | | gRNA 20 | AGGCATACTCATCTTTTTCAG | 37% |
| | | | (SEQ ID NO: 188) | |
| | | gRNA 21 | GGCATACTCATCTTTTTCAGT | 33% |
| | | | (SEQ ID NO: 189) | |
| | | gRNA 22 | CATACTCATCTTTTTCAGTGG | 29% |
| | | | (SEQ ID NO: 190) | |
| | | gRNA 23 | TCAGTAAGTCAACTTCAATGT | 41% |
| | | | (SEQ ID NO: 191) | |
| | | gRNA 26 | ACGTGAGTAAACCTGAATCTT | 22% |
| | | | (SEQ ID NO: 192) | |
| | ELANE | ELANEg35_ | AGTCCGGGCTGGGAGCGGGT | 49.5% |
| | g35 | OMNI-50 | (SEQ ID NO: 193) | |
| U2OS | ELANE | ELANEg35_ | AGTCCGGGCTGGGAGCGGGT | 95% |
| | g35 | OMNI-50 | (SEQ ID NO: 193) | |
| | ELANE | ELANEg38_ | ACAGCGGGTGTAGACTCCGA | 35% |
| | g38 | OMNI-50 | (SEQ ID NO: 194) | |
| | ELANE | ELANEg39_ | CAGCGGGTGTAGACTCCGAG | 75% |
| | g39 | OMNI-50 | (SEQ ID NO: 195) | |
| | ELANE | ELANEg58_ | GCTGCGGGAAAGGGATTCCC | 83% |
| | g58 | OMNI-50 | (SEQ ID NO: 196) | |
| | ELANE | ELANEg62_ | GTCAAGCCCCAGAGGCCACA | 86% |
| | g62 | OMNI-50 | (SEQ ID NO: 197) | |
| iPSC | ELANE | ELANEg35_ | AGTCCGGGCTGGGAGCGGGT | 53% |
| | g35 | OMNI-50 | (SEQ ID NO: 193) | |
| HSC | ELANE | ELANEg35_ | AGTCCGGGCTGGGAGCGGGT | 96% |
| | g35 | OMNI-50 | (SEQ ID NO: 193) | |
| | ELANE | ELANEg38_ | ACAGCGGGTGTAGACTCCGA | 44% |
| | g38 | OMNI-50 | (SEQ ID NO: 194) | |
| | ELANE | ELANEg39_ | CAGCGGGTGTAGACTCCGAG | 59% |
| | g39 | OMNI-50 | (SEQ ID NO: 195) | |
| | ELANE | ELANEg58_ | GCTGCGGGAAAGGGATTCCC | 22% |
| | g58 | OMNI-50 | (SEQ ID NO: 196) | |
| | ELANE | ELANEg62_ | GTCAAGCCCCAGAGGCCACA | 59% |
| | g62 | OMNI-50 | (SEQ ID NO: 197) | |

Table 7. OMNI-50 activity as RNP: OMNI-50 RNP was assembled with synthetic sgRNA (Agilent) and electroporated into cells. Several cell types were tested with a variety of sgRNAs. Cellular system, gene name, and spacer sequences are indicated next to the editing level as measured by NGS.

TABLE 8
OMNI-50 Multiplexing

| | | | OMNI-50 Editing | | OMNI-50 STD | |
|---|---|---|---|---|---|---|
| Gene | Site | Spacer Sequence | Donor 1 | Donor 2 | Donor 1 | Donor 2 |
| TRAC | gRNA 32 | GCCACTTTCAG | 59.00 | 53.00 | 9.00 | 10.00 |
| | | GAGGAGGATT | | | | |
| | | (SEQ ID NO: 177) | | | | |
| B2M | gRNA 15 | TCACAGCCCAA | 42.00 | 44.00 | 13.00 | 19.00 |
| | | GATAGTTAAG | | | | |
| | | (SEQ ID NO: 184) | | | | |
| TRAC + | gRNA 32 + | Test for TRAC | 55.00 | 44.00 | 5.00 | 1.00 |
| B2M | gRNA 15 | | | | | |
| TRAC + | gRNA 32 + | Test for B2M | 22.00 | 27.00 | 3.00 | 1.00 |
| B2M | gRNA 15 | | | | | |

Table 8. OMNI-50 multiplexing in primary T cells: Multiplexing of OMNI-50 was performed by electroporation into activated primary T cells, targeting either TRAC or B2M genes, or combined targeting. The first two rows show each gene separately on two donors that were randomly chosen from a five-donor bank. The final two rows show the same analysis for each gene when electroporation was performed as a multiplex. Editing activity was determined by indel count after amplicon based NGS. Standard deviation of duplicates is also shown. Using only TRAC gRNA had no effect on the B2M gene and vice versa (not shown).

TABLE 9
OMNI-50 ™ off-targets

| | | | | | SpCas9 on | SpCas9 | OMNI-50 ™ | OMNI-50 ™ |
|---|---|---|---|---|---|---|---|---|
| | | | | | target | ODN | on target | ODN |
| Guide | SpCas9 #1 | SpCas9 #2 | OMNI-50 ™ #1 | OMNI-50 ™ #2 | editing | integration | editing | integration |
| ELANE g35 | 206 | 201 | 11 | 5 | 85%, 90% | 58%, 62% | 97%, 97% | 37%, 38% |
| ELANE g58 | 51 | 92 | 18 | 13 | 86%, 86% | 39%, 42% | 88%, 83% | 37%, 36% |
| ELANE g58_alt | 67 | N.A. | 4 | 9 | 82% | 34% | 88%, 82% | 34%, 39% |
| ELANE g62 | 17 | 12 | 12 | 15 | 88%, 90% | 3%, 27% | 89%, 89% | 22%, 17% |
| ELANE g62_alt | 18 | 13 | 5 | 9 | 1%, 2% | N.A. | 0%, 0% | N.A. |
| TRAC g32 | 10 | 9 | 5 | 5 | 93%, 81% | 14% | 51%, 74% | 31%, 26% |

Table 9. OMNI-50 off-targets analysis by unbiased biochemical assay (guide seq): Off-target site counts of SpCas9 or OMNI-50 nucleases is shown in two replicates. For this analysis, only amplified sites with ≥10 reads were analyzed, and sites with a lower number of reads were discarded in order to reduce background noise. The editing level at the on-target site determined by indel count after amplicon based NGS is also indicated, as well as ODN integration.

TABLE 10
OMNI-50 ™ spacer optimization

| | U2OS cell line | | | | HSC | | | | iPSC | | | |
|---|---|---|---|---|---|---|---|---|---|---|---|---|
| | % editing | % editing | STD | | % editing | % editing | STD | | % editing | % editing | STD | |
| | ELANE | g35 | ELANE | STD | ELANE | g35 | ELANE | STD | ELANE | g35 | ELANE | STD |
| | g35 | Off-target | g35 | Off-target | g35 | Off-target | g35 | Off-target | g35 | Off-target | g35 | Off-target |
| 17bp | 0.54 | 0.20 | 0.28 | 0.04 | N.A. | N.A. | N.A. | N.A. | N.A. | N.A. | N.A. | N.A. |
| 18bp | 0.59 | 0.15 | 0.30 | 0.01 | N.A. | N.A. | N.A. | N.A. | N.A. | N.A. | N.A. | N.A. |
| 19bp | 3.20 | 0.21 | 0.29 | 0.07 | 11.54 | 0.00 | 3.24 | 0.00 | 0.41 | 0.00 | 0.19 | 0.00 |
| 20bp | 43.77 | 0.21 | 12.77 | 0.02 | 26.47 | 0.00 | 3.23 | 0.00 | 6.63 | 0.00 | 0.68 | 0.00 |
| 21bp | 91.50 | 0.37 | 4.66 | 0.03 | 74.86 | 0.00 | 1.70 | 0.00 | 48.15 | 0.00 | 0.16 | 0.00 |
| 22bp | 90.87 | 10.50 | 3.63 | 5.55 | 89.10 | 0.10 | 1.14 | 0.11 | 52.80 | 3.60 | 3.77 | 0.27 |
| 23bp | 75.81 | 7.59 | 8.35 | 4.25 | 85.86 | 0.10 | 1.69 | 0.27 | 51.16 | 2.50 | 7.40 | 0.15 |

Table 10. OMNI-50 spacer optimization. RNP was assembled for OMNI-50 protein with sgRNA of different lengths. The RNPs were electroporated into U2OS, HSCs, and iPSCs cells, and activity was determined by indel count after amplicon based NGS. OMNI-50 was tested on ELANE g35 in duplicates (standard deviation is shown). Table 10 Appendix shows a detailed comparison of 21 nt vs 22 nt spacer was done across five different genomic sites in HSCs.

TABLE 10
Appendix-comparison of 21nt vs 22nt spacers in HSCs

| | 21nt % | 22nt % | 21nt | 22nt |
|---|---|---|---|---|
| | editing | editing | STD | STD |
| ELANE g35 | 71.04 | 96.32 | 3.23 | 1.43 |
| ELANE g38 | 9.79 | 43.89 | 1.26 | 0.53 |
| ELANE g39 | 19.30 | 58.87 | 1.20 | 1.07 |
| ELANE g58 | 11.02 | 21.86 | 2.83 | 0.40 |
| ELANE g62 | 26.86 | 58.70 | 0.23 | 0.70 |

### REFERENCES

1. Ahmad and Allen (1992) “Antibody-mediated Specific Binging and Cytotoxicity of Lipsome-entrapped Doxorubicin to Lung Cancer Cells in Vitro”, Cancer Research 52:4817-20.
2. Anderson (1992) “Human gene therapy”, Science 256:808-13.
3. Basha et al. (2011) “Influence of Cationic Lipid Composition on Gene Silencing Properties of Lipid Nanoparticle Formulations of siRNA in Antigen-Presenting Cells”, Mol. Ther. 19(12):2186-200.
4. Behr (1994) “Gene transfer with synthetic cationic amphiphiles: Prospects for gene therapy”, Bioconjuage Chem 5:382-89.
5. Blaese et al. (1995) “Vectors in cancer therapy: how will they deliver”, Cancer Gene Ther.

2:291-97.
6. Blaese et al. (1995) “T lympocyte-directed gene therapy for ADA-SCID: initial trial results after 4 years”, Science 270(5235):475-80.
7. Briner et al. (2014) “Guide RNA functional modules direct Cas9 activity and orthognality”,

Molecular Cell 56:333-39.
8. Buchschacher and Panganiban (1992) “Human immunodeficiency virus vectors for inducible expression of foreign genes”, J. Virol. 66:2731-39.
9. Burstein et al. (2017) “New CRISPR-Cas systems from uncultivated microbes”, Nature 542:237-41.
10. Canver et al., (2015) “BCL11A enhancer dissection by Cas9-mediated in situ saturating mutagenesis”, Nature Vol. 527, Pgs. 192-214.
11 Chang and Wilson (1987) “Modification of DNA ends can decrease end-joining relative to homologous recombination in mammalian cells”, Proc. Natl. Acad. Sci. USA 84:4959-4963.
12. Charlesworth et al. (2019) “Identification of preexisting adaptive immunity to Cas9 proteins in humans”, Nature Medicine, 25(2), 249.
13. Chung et al. (2006) “Agrobacterium is not alone: gene transfer to plants by viruses and other bacteria”, Trends Plant Sci. 11(1):1-4.
14. Coelho et al. (2013) “Safety and efficacy of RNAi therapy for transthyretin amyloidosis” N. Engl. J. Med. 369, 819-829.
15. Crystal (1995) “Transfer of genes to humans: early lessons and obstacles to success”, Science 270(5235):404-10.
16. Dillon (1993) “Regulation gene expression in gene therapy” Trends in Biotechnology 11(5):167-173.
17. Dranoff et al. (1997) “A phase I study of vaccination with autologous, irradiated melanoma cells engineered to secrete human granulocyte macrophage colony stimulating factor”, Hum. Gene Ther. 8(1):111-23.
18. Dunbar et al. (1995) “Retrovirally marked CD34-enriched peripheral blood and bone marrow cells contribute to long-term engraftment after autologous transplantation”, Blood 85:3048-57.
19. Ellem et al. (1997) “A case report: immune responses and clinical course of the first human use of ganulocyte/macrophage-colony-stimulating-factor-tranduced autologous melanoma cells for immunotherapy”, Cancer Immunol Immunother 44:10-20.
20. Gao and Huang (1995) “Cationic liposome-mediated gene transfer” Gene Ther. 2(10):710-22.
21. Haddada et al. (1995) “Gene Therapy Using Adenovirus Vectors”, in: The Molecular Repertoire of Adenoviruses III: Biology and Pathogenesis, ed. Doerfler and Bohm, pp. 297-306.
22. Han et al. (1995) “Ligand-directed retro-viral targeting of human breast cancer cells”, Proc. Natl. Acad. Sci. USA 92(21):9747-51.
23. Humbert et al., (2019) “Therapeutically relevant engraftment of a CRISPR-Cas9—edited HSC-enriched population with HbF reactivation in nonhuman primates”, Sci. Trans. Med., Vol. 11, Pgs. 1-13.
24. Inaba et al. (1992) “Generation of large numbers of dendritic cells from mouse bone marrow cultures supplemented with granulocyte/macrophage colony-stimulating factor”, J Exp Med. 176(6):1693-702.
25. Jiang and Doudna (2017) “CRISPR-Cas9 Structures and Mechanisms”, Annual Review of Biophysics 46:505-29.
26. Jinek et al. (2012) “A programmable dual-RNA-guided DNA endonuclease in adaptive bacterial immunity”, Science 337(6096):816-21.
27. Johan et al. (1992) “GLVR1, a receptor for gibbon ape leukemia virus, is homologous to a phosphate permease of Neurospora crassa and is expressed at high levels in the brain and thymus”, J Virol 66(3):1635-40.
28. Judge et al. (2006) “Design of noninflammatory synthetic siRNA mediating potent gene silencing in vivo”, Mol Ther. 13(3):494-505.
29. Kohn et al. (1995) “Engraftment of gene-modified umbilical cord blood cells in neonates with adnosine deaminase deficiency”, Nature Medicine 1:1017-23.
30. Kremer and Perricaudet (1995) “Adenovirus and adeno-associated virus mediated gene transfer”, Br. Med. Bull. 51(1):31-44.
31. Macdiarmid et al. (2009) “Sequential treatment of drug-resistant tumors with targeted minicells containing siRNA or a cytotoxic drug”, Nat Biotehcnol. 27(7):643-51.
32. Malech et al. (1997) “Prolonged production of NADPH oxidase-corrected granulocyes after gene therapy of chronic granulomatous disease”, PNAS 94(22):12133-38.
33. Maxwell et al. (2018) “A detailed cell-free transcription-translation-based assay to decipher CRISPR protospacer adjacent motifs”, Methods 14348-57
34. Miller et al. (1991) “Construction and properties of retrovirus packaging cells based on gibbon ape leukemia virus”, J Virol. 65(5):2220-24.
35. Miller (1992) “Human gene therapy comes of age”, Nature 357:455-60.
36. Mir et al. (2019) “Type II-C CRISPR-Cas9 Biology, Mechanism and Application”, ACS Chem. Biol. 13(2):357-365.
37. Mitani and Caskey (1993) “Delivering therapeutic genes—matching approach and application”, Trends in Biotechnology 11(5):162-66.
38. Nabel and Felgner (1993) “Direct gene transfer for immunotherapy and immunization”, Trends in Biotechnology 11(5):211-15.
39. Nehls et al. (1996) “Two genetically separable steps in the differentiation of thymic epithelium” Science 272:886-889.
40. Nishimasu et al. “Crystal structure of Cas9 in complex with guide RNA and target DNA” (2014) Cell 156(5):935-49.
41. Nishimasu et al. (2015) “Crystal Structure of Staphylococcus aureus Cas9” Cell 162(5):1113-26.
42. Palermo et al. (2018) “Key role of the REC lobe during CRISPR-Cas9 activation by ‘sensing’, ‘regulating’, and ‘locking’ the catalytic HNH domain” Quarterly Reviews of Biophysics 51, e9, 1-11.
43. Remy et al. (1994) “Gene Transfer with a Series of Lipphilic DNA-Binding Molecules”, Bioconjugate Chem. 5(6):647-54.
44. Sentmanat et al. (2018) “A Survey of Validation Strategies for CRISPR-Cas9 Editing”, Scientific Reports 8:888, doi:10.1038/s41598-018-19441-8.
45. Sommerfelt et al. (1990) “Localization of the receptor gene for type D simian retroviruses on human chromosome 19”, J. Virol. 64(12):6214-20.
46. Van Brunt (1988) “Molecular framing: transgenic animals as bioactors” Biotechnology 6:1149-54.
47. Vigne et al. (1995) “Third-generation adenovectors for gene therapy”, Restorative Neurology and Neuroscience 8(1,2): 35-36.
48. Wagner et al. (2019) “High prevalence of Streptococcus pyogenes Cas9-reactive T cells within the adult human population” Nature Medicine, 25(2), 242
49. Wilson et al. (1989) “Formation of infectious hybrid virion with gibbon ape leukemia virus and human T-cell leukemia virus retroviral envelope glycoproteins and the gag and pol proteins of Moloney murine leukemia virus”, J. Virol. 63:2374-78.
50. Yu et al. (1994) “Progress towards gene therapy for HIV infection”, Gene Ther. 1(1):13-26.
51. Zetsche et al. (2015) “Cpf1 is a single RNA-guided endonuclease of a class 2 CRIPSR-Cas system” Cell 163(3):759-71.
52. Zuris et al. (2015) “Cationic lipid-mediated delivery of proteins enables efficient protein based genome editing in vitro and in vivo” Nat Biotechnol. 33(1):73-80.

1-38. (canceled)

39. A non-naturally occurring composition comprising a CRISPR nuclease,
wherein the CRISPR nuclease comprises a Domain A which comprises at least one of
a) Subdomain A1 having at least 97% sequence identity to amino acids 1 to 50 of SEQ ID NO: 3;
b) Subdomain A2 having at least 97% sequence identity to amino acids 741 to 789 of SEQ ID NO: 3; or
c) Subdomain A3 having at least 97% sequence identity to amino acids 962 to 1096 of SEQ ID NO: 3; and/or
wherein the CRISPR nuclease comprises a Domain B having at least 97% sequence identity to amino acids 51 to 83 of SEQ ID NO: 3; and/or
wherein the CRISPR nuclease comprises a Domain C
which comprises at least one of
a) Subdomain C1 having at least 97% sequence identity to amino acids 84 to 160 of SEQ ID NO: 3;
b) Subdomain C2 having at least 97% sequence identity to amino acids 161 to 299 of SEQ ID NO: 3; or
c) Subdomain C3 having at least 97% sequence identity to amino acids 300 to 737 of SEQ ID NO: 3; or
which comprises at least one of
a) Subdomain Ca having at least 97% sequence identity to amino acids 84 to 478 of SEQ ID NO: 3; or
b) Subdomain Cb having at least 97% sequence identity to amino acids 479 to 737 of SEQ ID NO: 3; or
has at least 97% sequence identity to amino acids 84 to 737 of SEQ ID NO: 3; and/or
wherein the CRISPR nuclease comprises a Domain D having at least 97% sequence identity to amino acids 790 to 961 of SEQ ID NO: 3; and/or
wherein the CRISPR nuclease comprises a Domain E having at least 97% sequence identity to amino acids 1097 to 1196 of SEQ ID NO: 3; and/or
wherein the CRISPR nuclease comprises a Domain F having at least 97% sequence identity to amino acids 1197 to 1370 of SEQ ID NO: 3.

40. The composition of claim 39, wherein the CRISPR nuclease comprises a Domain A which comprises at least one of
a) Subdomain A1 having at least 97% sequence identity to amino acids 1 to 50 of SEQ ID NO: 3;
b) Subdomain A2 having at least 97% sequence identity to amino acids 741 to 789 of SEQ ID NO: 3; or
c) Subdomain A3 having at least 97% sequence identity to amino acids 962 to 1096 of SEQ ID NO: 3.

41. The composition of claim 39, wherein the CRISPR nuclease comprises a Domain B having at least 97% sequence identity to amino acids 51 to 83 of SEQ ID NO: 3.

42. The composition of claim 39, wherein the CRISPR nuclease comprises a Domain C which comprises at least one of
a) Subdomain C1 having at least 97% sequence identity to amino acids 84 to 160 of SEQ ID NO: 3;
b) Subdomain C2 having at least 97% sequence identity to amino acids 161 to 299 of SEQ ID NO: 3; or
c) Subdomain C3 having at least 97% sequence identity to amino acids 300 to 737 of SEQ ID NO: 3.

43. The composition of claim 39, wherein the CRISPR nuclease comprises a Domain C which comprises at least one of
a) Subdomain Ca having at least 97% sequence identity to amino acids 84 to 478 of SEQ ID NO: 3; and
b) Subdomain Cb having at least 97% sequence identity to amino acids 479 to 737 of SEQ ID NO: 3.

44. The composition of claim 39, wherein Domain C has at least 97% sequence identity to amino acids 84 to 737 of SEQ ID NO: 3.

45. The composition of claim 39, wherein the CRISPR nuclease comprises a Domain D having at least 97% sequence identity to amino acids 790 to 961 of SEQ ID NO: 3.

46. The composition of claim 39, wherein the CRISPR nuclease comprises a Domain E having at least 97% sequence identity to amino acids 1097 to 1196 of SEQ ID NO: 3.

47. The composition of claim 39, wherein the CRISPR nuclease comprises a Domain F having at least 97% sequence identity to amino acids 1197 to 1370 of SEQ ID NO: 3.

48. The composition of claim 39, wherein the CRISPR nuclease comprises Domain A, Domain B, Domain C, Domain D, Domain E, and Domain F, wherein
a) Domain A comprises
i) Subdomain A1 having at least 97% sequence identity to amino acids 1 to 50 of SEQ ID NO: 3;
ii) Subdomain A2 having at least 97% sequence identity to amino acids 741 to 789 of SEQ ID NO: 3; and
iii) Subdomain A3 having at least 97% sequence identity to amino acids 962 to 1096 of SEQ ID NO: 3;
b) Domain B has at least 97% sequence identity to amino acids 51 to 83 of SEQ ID NO: 3;
c) Domain C has at least 97% sequence identity to amino acids 84 to 737 of SEQ ID NO: 3;
d) Domain D has at least 97% sequence identity to amino acids 790 to 961 of SEQ ID NO: 3;
e) Domain E has at least 97% sequence identity to amino acids 1097 to 1196 of SEQ ID NO: 3; and
f) Domain F has at least 97% sequence identity to amino acids 1197 to 1370 of SEQ ID NO: 3.

49. The composition of claim 39, wherein the CRISPR nuclease sequence is at least 100-250, 250-500, 500-1000, or 1000-2000 amino acids in length.

50. A non-naturally occurring composition comprising a peptide, wherein the peptide comprises an amino acid sequence having at least 97% sequence identity to the amino acid sequence of at least one of Domain A, Domain B, Domain C, Domain D, Domain E, or Domain F of the amino acid sequence of SEQ ID NO: 3.

51. A non-naturally occurring composition comprising a polynucleotide molecule encoding an amino acid sequence having at least 97% sequence identity to the amino acid sequence of at least one of Domain A, Domain B, Domain C, Domain D, Domain E, or Domain F of the amino acid sequence of SEQ ID NO: 3.

52. (canceled)

53. A method of modifying a nucleotide sequence at a target site in a cell-free system or the genome of a cell comprising introducing into the cell the composition of claim 39.

54. The method of claim 53, wherein the cell is a eukaryotic cell, a mammalian cell, or a plant cell.

55. (canceled)

56. A method of treating subject having a mutation disorder comprising targeting the composition of claim 39 to an allele associated with the mutation disorder.

57. The method of claim 56, wherein the mutation disorder is related to a disease or disorder selected from any of a beta thalassemia, sickle cell anemia, neoplasia, age-related macular degeneration, schizophrenia, neurological, neurodegenerative, or movement disorder, Fragile X Syndrome, secretase-related disorders, prion-related disorders, ALS, addiction, autism, Alzheimer's Disease, neutropenia, inflammation-related disorders, Parkinson's Disease, blood and coagulation diseases and disorders, cell dysregulation and oncology diseases and disorders, inflammation and immune-related diseases and disorders, metabolic, liver, kidney and protein diseases and disorders, muscular and skeletal diseases and disorders, dermatological diseases and disorders, neurological and neuronal diseases and disorders, and ocular diseases and disorders, or wherein the allele associated with the disease is BCL11A.

58-59. (canceled)

60. The composition of claim 39, further comprising a
a) a CRISPR RNA (crRNA) molecule and a transactivating CRISPR RNA (tracrRNA) molecule,
wherein the crRNA molecule, tracrRNA molecule, and the CRISPR nuclease do not naturally occur together; or
b) a single-guide RNA (sgRNA) molecule,
wherein the crRNA molecule or sgRNA molecule comprises a guide sequence portion that is complementary to a sequence in a target region.

61. The composition of claim 39, wherein the CRISPR nuclease further comprises a nuclear localization sequence (NLS).

62. The composition of claim 39, further comprising a donor template molecule.


SEQUENCE LISTING (197 sequences)

SEQ ID NO: 1 - protein, 1100 aa, Butyrivibrio sp. AC2005
MGYTIGLDLGVASLGWAVVNDEYEVLESCSNIFPAAESANNVERRGFRQGRRLSRRRRTRISDFRKLWEKSGFEVPSNELNEVLQYRIKGMNDKLSEDELYHVLLNSLKHRGISYLDDADDENASGDYAASIAYNENQLKTKLPCEIQWERYKKYGAYRGNITIQEGGEPLTLRNVFTTSAYEKEIQKLLDVQSMSNEKVTKKFIDEYLKIFSRKREYYIGPGNKKSRTDYGVYTTQKNEDGTYHTEQNLFDKLIGKCSVYPDERRAAGATYTAQEFNLLNDLNNLVIDGRKLDEQEKCQIVDAVKHAKTVNMKNIIAKVIGTKANSMNMTGARIDKNEKEIFHSFEAYNKLRKALEEIDFDIETLSTDELDAIGEVLTLNTDRKSIQNGLQEKRIVVPDEVRDVLIATRKRNGSLFSKWQSFGIRIMKELIPELYAQPKNQMQLLTDMGVFKTKDERFVEYDKIPSDLITEEIYNPVVAKTVRITVRVLNALIKKYGYPDRVVIEMPRDKNSEEEKKRIADFQKNNENELGGIIKKVKSEYGIEITDADFKNHSKLGLKLRLWNEQNETCPYSGKHIKIDDLLNNPNMFEVDHIIPLSISFDDSRANKVLVYAAENQNKGNRTPMAYLSNVNREWDFHEYMSFVLSNYKGTIYGKKRDNLLFSEDIYKIDVLQGFISRNINDTRYASKVILNSLQSFFGSKECDTKVKVVRGTFTHQMRMNLKIEKNREESYVHHAVDAMLIAFSQMGYDAYHKLTEKYIDYEHGEFVDQKGYEKLIENDVAYRETTYQNKWMTIKKNIEIAAEKNKYWYQVNRKSNRGLCNQTIYGTRNLDGKTVKISKLDIRTDDGIKKFKGIVEKGKLERFLMYRNDPKTFEWLLQIYKDYSDSKNPFVQYESETGDVIKKVSKTNNGPKVCELRYEDGEVGSCIDISHKYGYKKGSKKVILDSLNPYRMDVYYNTKDNRYYFVGVKYSDIKCQGDSYVIDEDKYAAALVQEKIVPEGKGRSDLTELGYEFKLSFYKNEIIEYEKDGEIYVERFLSRTMPKVSNYIETKPLEAAKFEKRNLVGLAKTSRIRKIRVDILGNRYLNSMENFDFVVGHK

SEQ ID NO: 2 - protein, 1104 aa, bacterium LF-3
MSRYVLGLDIGITSVGYGVIDIDNNLFVDYGVRLFKEGTAAENETRRTKRGSRRLKRRKSNRLNDMKNLLKENDLYFEDYRNYNPYEIRAKGLKEKLLPEELCTAIMHITKSRGTTLEALADESQDDEGTKATLSKNAKELNDGKYICEVQLDRLNKDHKVRGTENNFKTEDYVKELKEILKHQDLNEELCDQIIEMVSRRRRYDQGPGSEKSPTPYGSYRMVDGVLKHVNLIDEMRGRCSVYPDEFRAPKQSYTAELFNLLNDLNNLTIKGEKITVEEKEKVVAFVNEKGSITVKQLLKLLDAQEDEVTGFRIDKNDKPLITEFKGYSKVLKVFKKYNQQELLEDKLIVDQVIDICTKSKGIDERKKDIKELYPEFDNELIEELASVKGVSAYHSLSFKAMHIINKEMLTTEMNQIQVLHEIEMFDKNRKSLKGKKNIEPDEEAILSPVAKRAHRETFKVINALRKQYGEFDSIVIEMTRDKNSKEQVKRINDSQKRFKSENDRVDGIIKNSGIDPERVNGKTKTKIRLYLQQDCKTAYTQQDIDLHTLIFDDKAYEIDHIIPISVSLDDSLTNKVLASRLENQQKGNLTPMMAYLKGKFTGGNLEKYKLFVSSNKNFNGKKRNNLLTEQDITKEDVARKFINRNLVDTSYACRTVLNTLQRYFKDNEIDTKVHTIRGQSTNIFRKRINLQKDREQDYFHHAIDALIVASLKKMNIVNSYLMHYNYSDLYDEETGEVFDVLPDKQFIDQRYISFISDLKNIYQESNQYNLGYITQEQMHYPLIKVSHKIDTKPNRKIADETIYSTRNIEGQDMLVEKIKNIYDPKEKKAIELVNNIINDDTDKYIMKHKDPQTFEKIKEVVLNHFNDYKDSKEYYVIDKKGKYSLKEESPLTSYYNENGAITKYSKKNNGPAITSMKFYSEKLGNHLAITSNYNTNNKKVILKQISPYRTDFYVSPEGKYKFVTVRYKDVFYKETIHKFVIDENWYHEEKIKKGILEDWKFVCSMHRDELIGLIKPEGKKFVYDASINGGQTQYHDGKHYEILKFTATNDEKKRTFEVKPINTNCSKRLMPSVGPFIKIQKFATDVLGNIYEVKDNRLKLEFD

SEQ ID NO: 3 - protein, 1370 aa, Ezakiella peruensis strain M6.X2
MTKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILENLLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNQLLKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGALFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKL

SEQ ID NO: 4 - protein, 1369 aa, Clostridium sp. AF02-29
MKEKMEYYLGLDMGTNSVGWAVTDKEYRLMRAKGKDLWGVRLFERANTAEERRAYRINRRRRQREVARIGILKELFADEIAKVDANFFARLDDSKYYLDDRQENNKQKYAIFADKDYTDKEYFSQYQTIFHLRKELILSDQPHDVRLIYLALLNMFKHRGHFLNKTLGTSESLESFFDMYQRLAVCADGEGIKLPETVDLKKLEQILGARGCSRKATLEHISEIMGINKKNKPVYSLMQMICGLDTKMIDLFGQKIDEEHKKISLSFRTSNYEEMAEEVRNTIGDDAFELILTAKEMHDFGLLAEIMKGYSYLSEARVAVYEEHRKDLAKLKAVFKQYDHKAYDEMFRIMKNGTYSAYVGSVNSFGKIERRTVKTSREELLKNIKKILTGFPEDDATVQEFLGKIDSDTLLQKQLTASNGVIPNQVHAKEMKVILKNAEKYLPFLSERDETGLSVSEKIIALFTFTIPYYVGPLGQQHLGKECAHGWVERKEKGTVYPWNFEQKVDLKASAEHFIERMVKHCTYLSDEQALPKQSLLYEKFQVLNELNNLKIRGEKISVELKQQIYRDVFEHTGKKVSMKQLENYLKLNGLLEKDEKDAVTGIDGGFHSYLSSLGKFIGILGEEAHYGKNQNMMEKIVFWGTVYGQDKKFLRERLSEVYGDRLSKEQIRRITGMKFEGWGRLSKEFLLLEGASREEGEIRTLIRSLWETNENLMGLLSERYTYSEEVREKTLECEKSLSEWTIEDLEGMYLSAPVKRMVWQTLLIVKELEKVLGCAPRRIFVEMAREDAEKGRRTESRKQKLQNLYKAIKKEEIDWKKEIDEKTEQAFRSKKLYLYYLQKGRCMYTGESIRFEDLMNDNLYDIDHIYPRHFVKDDSLEQNLVLVKKEKNAHKSDVFPIEADIQKKMSPFWKELKERGFISEEKYMRLTRRYGFSEEEKAGFINRQLVETRQGTKSITEILGQAFPDVDIIFSKASNVSEFRHIYGLYKVRSINDFHHAHDAYLNIVVGNTYHVKFTKNPLNFIREAEKNPQNAENKYNMNRMFDWTVKRGNETAWIASSDKEAGSIKIVKAILAKNTPLVTKRCAEAHGGITRKATIWNKNKAAGSGYIPVKMNDARLLDVTKYGGLTSVSASGYTLLEYDVKGKKIRSLEAIPIYLGRVSELTNEAILKYFEKVLIEENKGKEITELRICKKFIPRESLVRYNGYYYYLGGKSVEQIVLKNATQMAYSEEETCYIKKIEKAIEKTYYEEVDKNKNVILTKTRNNAMYDKFIIKYQNSIYQNQSGAMKNSIIGKRNEFLTLSLEKQCRILKALVEYFRTGDIIDLRELGGSSQAGKVAMNKKIMGASELVLISQSPTGLFQQEIDLLKI

SEQ ID NO: 5 - DNA, 3303 bp, Butyrivibrio sp. AC2005
  source 1..3303: mol_type = other DNA
atgggatatacaataggacttgatcttggtgtggcttcattaggatgggctgtagtcaatgatgaatatgaggtattagaatcatgctcaaatatttttcctgcagcagaatctgcaaataatgttgaaagacgaggctttaggcagggaagaaggttgtcaaggcgtcgcaggaccagaattagtgatttcagaaaactgtgggagaagagtggtttcgaggttccttcaaatgaattgaacgaggtgcttcagtataggattaaaggcatgaatgataaattatcagaagatgagctttatcatgttcttttaaatagcctgaaacataggggaatttcgtatttggatgatgcagatgatgaaaatgcatctggggattatgctgcaagcattgcttataacgaaaatcaattaaagacaaaattgccttgtgagattcagtgggagcgctataagaaatatggtgcttatagggggaatattactatccaagaaggtggggaaccgcttactcttagaaatgtattcacaacaagtgcgtatgaaaaagaaattcagaagctattagacgtacaatctatgtcaaatgagaaagtaacaaaaaagtttattgatgaatacttaaaaatcttttcaagaaaaagagaatattatattgggccgggtaacaaaaaatccagaacagattatggtgtatacactacacaaaaaaatgaagatggtacttatcatactgagcagaatctttttgataaattgattggaaagtgtagtgtatatcctgatgagagaagagctgccggggctacttatactgcacaggaatttaatcttttaaatgatctgaataatcttgtaattgatggaagaaaactagatgagcaggaaaaatgtcagattgttgatgctgttaaacatgctaaaaccgtcaatatgaagaacattattgcaaaagtcattggaacaaaagcaaactcaatgaatatgaccggcgcaagaatagataagaatgaaaaagaaatttttcattcttttgaggcttataacaagttaagaaaagcactggaagaaatagattttgatatagagactttgtctacggatgagttggatgctataggagaagtgttgactcttaatactgaccgaaaatcaattcaaaacggacttcaagagaaaagaatagtagttcctgatgaagtcagggatgtgcttatcgcaaccaggaaaagaaatggctcattatttagcaaatggcagtcatttggtataagaatcatgaaggaattgattcctgaattatatgcgcagcctaagaatcagatgcaactgcttactgatatgggagtatttaaaactaaggatgagagatttgttgagtatgataagattccgtctgatctaataacagaagaaatctataatcctgtggttgctaaaactgtaaggattactgtcagagttttgaatgctcttattaagaaatatggctatccggatagagttgttatagagatgccaagagataaaaactcagaagaagagaaaaagcgcatagcagattttcaaaagaacaatgagaatgagcttggtggaataataaaaaaagtaaagtcagaatatggtattgaaataactgatgcggattttaagaaccatagtaaacttggacttaaacttaggttgtggaatgaacagaatgaaacatgtccttactcagggaaacatataaagattgatgaccttttaaataatcctaatatgtttgaggtggatcatattatcccattatccatttcatttgatgatagtagagccaataaagtgttggtatacgctgctgaaaatcagaataagggtaacagaacgccaatggcatacctgtccaatgttaatagagaatgggatttccatgaatacatgagttttgttcttagtaattataagggaacaatatatggtaagaagagagataatcttttattctcagaggacatatataaaattgatgttttacagggatttattagcagaaatataaatgatacaagatatgcttcaaaggtaatacttaattcattacagtctttctttggttcaaaagagtgcgacacgaaggtgaaggttgttagaggaacctttacacatcagatgcgaatgaatctaaagatagaaaagaatagagaggagtcatatgtgcatcatgctgttgatgctatgcttatagctttttctcaaatggggtatgatgcatatcataaacttacagagaagtatattgattatgaacatggcgaatttgtagatcagaaaggctatgagaagcttattgaaaatgatgtagcatatcgtgaaaccacttatcaaaataagtggatgactataaagaaaaatatagaaatagcagctgaaaagaataaatactggtatcaggtaaataggaaaagcaatagagggctttgcaaccagactatttatggtaccagaaatctggatggcaagacagtaaagatcagcaaacttgatattcggacagatgatgggataaagaaatttaaagggatcgtagaaaaaggtaaactagaacgctttttgatgtataggaatgatccaaaaacatttgaatggctgcttcagatttataaggattattcagactccaaaaacccatttgtccaatatgaatcagagactggtgatgttattaagaaagtttcaaaaacgaataatggaccaaaggtatgtgaacttcgctatgaagatggtgaggttggtagctgtatcgatatttctcataagtatggatataaaaagggtagtaaaaaggtaattctcgattctttaaacccttacagaatggatgtatattataacactaaggacaataggtattattttgttggtgtaaagtattcagacattaagtgccaaggtgatagctatgtaatcgatgaggataaatacgcagcagcactcgttcaggaaaaaatagtgccggaaggaaaaggaagaagtgacttaacagagcttggttatgaatttaagctatcattttataaaaatgagataatagagtatgaaaaagatggcgaaatatatgtagaaagatttttatcgcgaacaatgccaaaagtgagcaattatattgaaactaagccattggaagctgcaaaatttgaaaaacgaaatttagtggggttagctaagactagcagaataagaaaaatacgagtggatatacttgggaatcgttatttaaatagtatggaaaatttcgattttgttgtgggacataaataa

SEQ ID NO: 6 - DNA, 3297 bp, synthetic construct
  misc_feature 1..3297: note = Synthetic
  source 1..3297: mol_type = other DNA
gggtacaccattggcttggatttgggagtggcttcattgggttgggcagtcgtgaacgacgagtacgaagtgctcgagtcttgtagcaacatcttccccgccgccgagtccgctaacaacgtcgagcgaagagggttccgccaaggcaggcggttgtctcggcgcaggcgcactcgtataagcgattttcgtaagctttgggaaaagagcggatttgaagtgcccagtaacgagctgaatgaagttctccaataccggatcaaggggatgaacgacaagctgagtgaggacgaattgtaccacgtgctgttgaactcattgaagcaccggggtatcagctacctggacgacgccgacgacgagaacgcctcaggtgactacgccgcctctatcgcgtacaatgagaaccagttgaaaaccaagctcccctgcgaaatccaatgggaaaggtacaagaagtacggggcgtaccgcggtaacatcaccatacaggagggaggcgagccactgactctccgaaacgtgtttacgacgtctgcttacgagaaggagatccagaaactcttggatgtgcagagtatgagtaacgaaaaggtcacgaagaaattcatcgacgagtatctgaagattttcagtcgcaagagggagtactacataggtccaggcaataagaagtcacgaaccgactacggcgtttataccactcagaagaacgaggacggcacctaccacacagaacaaaacctgttcgacaagcttatcggtaaatgctccgtttaccccgacgaaaggcgcgcagcgggtgccacatacacagcccaagagttcaacttgctgaacgacttgaacaacctcgttatcgacggcaggaagctggacgaacaagagaagtgccaaatcgtcgacgcggtgaagcacgccaagacggttaacatgaagaatatcatcgccaaggtaatcggtactaaggcgaatagtatgaacatgacaggggctaggattgacaagaacgagaaggagatcttccacagtttcgaagcgtacaataaactgaggaaggctctcgaggagattgacttcgacattgaaaccctcagtaccgacgaacttgacgccatcggggaagtcctgacactgaacaccgatagaaagagcatccagaatgggttgcaggaaaagcggatcgtggtccccgacgaggtaagagatgtactgattgccactcgtaagcgtaacgggagcctgttctccaagtggcaatctttcggaatccgtattatgaaagagctcatcccggagctgtacgcccaaccaaagaaccaaatgcagttgctgaccgacatgggcgtcttcaagaccaaagacgaacggttcgtggaatacgacaaaatccccagtgacctcatcacggaagagatatacaaccccgttgtcgccaagaccgtccgcatcaccgttcgcgtccttaacgcgctcatcaagaagtacgggtatcccgacagggtggtgatcgaaatgcctcgtgacaagaatagtgaggaagaaaagaaaaggattgctgacttccagaagaataacgaaaacgaactgggcggcatcatcaagaaggtcaaaagtgagtacggcatcgagatcaccgacgcagacttcaagaatcacagcaagttgggtctcaagctgcgactctggaacgagcaaaacgagacttgtccctatagcggcaagcacattaaaatcgacgatctgttgaacaacccgaacatgttcgaagtagaccacatcattcccctctcaatctccttcgacgactctcgcgctaacaaggtcctggtgtatgcagcagagaaccaaaacaaaggaaataggactcccatggcttatttgagtaacgtcaaccgcgagtgggactttcacgagtatatgtctttcgtgctgtcaaactacaaaggcactatctacgggaagaaacgggacaacctcttgttttccgaagatatctacaagatagacgtgctgcaagggttcatctcccggaacatcaacgacacccgatacgcgagtaaagtgattctgaacagcctgcaaagtttcttcgggtctaaggaatgtgataccaaagtcaaagtggtacggggcactttcacgcaccaaatgagaatgaacttgaaaattgagaagaaccgggaagaaagttacgtccaccacgcagtcgacgcaatgctgattgccttcagccagatgggctacgacgcctaccacaagctcaccgagaaatacatagactacgagcacggagagttcgtggaccaaaagggatacgaaaagctgatcgagaacgacgtcgcctacagggaaacgacctaccagaacaaatggatgacaatcaagaagaacattgagatcgctgccgagaagaacaagtattggtatcaagtgaaccggaagtcaaacaggggactgtgtaatcaaaccatctacggcactcgtaaccttgacgggaaaaccgtgaaaatttctaagctcgacatccgcactgacgacggaatcaagaagttcaagggtattgttgagaagggcaagcttgagagattccttatgtaccgtaacgaccctaagaccttcgagtggctcctgcaaatctacaaagactactctgatagcaagaatcccttcgtgcagtacgagtccgaaacaggtgacgtgataaagaaggtaagcaagacaaacaacggccccaaagtctgcgagctgcgatacgaggacggggaagtgggaagttgcattgacatatcccacaaatacgggtacaagaaaggcagcaagaaagtgatcctggacagcctgaatccctatcgcatggacgtgtactacaataccaaagataacagatactacttcgtgggcgttaaatactctgatatcaaatgtcagggagactcttacgtgattgacgaagacaagtatgctgctgccctggtacaagagaagatcgtacctgaggggaaggggcgcagcgatctcactgaactgggctacgagttcaaactgtctttctacaagaacgaaattattgaatacgagaaggacggggagatctacgtcgagcgcttcctgtcaaggaccatgcccaaggtctccaactacatcgagacaaaaccccttgaggccgctaagttcgagaagcggaacctggtaggattggccaaaacatcaaggattcgaaagattagagtcgacattctcggcaacaggtatctgaactcaatggagaactttgacttcgtcgttggtcacaag

SEQ ID NO: 7 - DNA, 3300 bp, synthetic construct
  misc_feature 1..3300: note = Synthetic
  source 1..3300: mol_type = other DNA
atggggtacaccattggcttggatttgggagtggcttcattgggttgggcagtcgtgaacgacgagtacgaagtgctcgagtcttgtagcaacatcttccccgccgccgagtccgctaacaacgtcgagcgaagagggttccgccaaggcaggcggttgtctcggcgcaggcgcactcgtataagcgattttcgtaagctttgggaaaagagcggatttgaagtgcccagtaacgagctgaatgaagttctccaataccggatcaaggggatgaacgacaagctgagtgaggacgaattgtaccacgtgctgttgaactcattgaagcaccggggtatcagctacctggacgacgccgacgacgagaacgcctcaggtgactacgccgcctctatcgcgtacaatgagaaccagttgaaaaccaagctcccctgcgaaatccaatgggaaaggtacaagaagtacggggcgtaccgcggtaacatcaccatacaggagggaggcgagccactgactctccgaaacgtgtttacgacgtctgcttacgagaaggagatccagaaactcttggatgtgcagagtatgagtaacgaaaaggtcacgaagaaattcatcgacgagtatctgaagattttcagtcgcaagagggagtactacataggtccaggcaataagaagtcacgaaccgactacggcgtttataccactcagaagaacgaggacggcacctaccacacagaacaaaacctgttcgacaagcttatcggtaaatgctccgtttaccccgacgaaaggcgcgcagcgggtgccacatacacagcccaagagttcaacttgctgaacgacttgaacaacctcgttatcgacggcaggaagctggacgaacaagagaagtgccaaatcgtcgacgcggtgaagcacgccaagacggttaacatgaagaatatcatcgccaaggtaatcggtactaaggcgaatagtatgaacatgacaggggctaggattgacaagaacgagaaggagatcttccacagtttcgaagcgtacaataaactgaggaaggctctcgaggagattgacttcgacattgaaaccctcagtaccgacgaacttgacgccatcggggaagtcctgacactgaacaccgatagaaagagcatccagaatgggttgcaggaaaagcggatcgtggtccccgacgaggtaagagatgtactgattgccactcgtaagcgtaacgggagcctgttctccaagtggcaatctttcggaatccgtattatgaaagagctcatcccggagctgtacgcccaaccaaagaaccaaatgcagttgctgaccgacatgggcgtcttcaagaccaaagacgaacggttcgtggaatacgacaaaatccccagtgacctcatcacggaagagatatacaaccccgttgtcgccaagaccgtccgcatcaccgttcgcgtccttaacgcgctcatcaagaagtacgggtatcccgacagggtggtgatcgaaatgcctcgtgacaagaatagtgaggaagaaaagaaaaggattgctgacttccagaagaataacgaaaacgaactgggcggcatcatcaagaaggtcaaaagtgagtacggcatcgagatcaccgacgcagacttcaagaatcacagcaagttgggtctcaagctgcgactctggaacgagcaaaacgagacttgtccctatagcggcaagcacattaaaatcgacgatctgttgaacaacccgaacatgttcgaagtagaccacatcattcccctctcaatctccttcgacgactctcgcgctaacaaggtcctggtgtatgcagcagagaaccaaaacaaaggaaataggactcccatggcttatttgagtaacgtcaaccgcgagtgggactttcacgagtatatgtctttcgtgctgtcaaactacaaaggcactatctacgggaagaaacgggacaacctcttgttttccgaagatatctacaagatagacgtgctgcaagggttcatctcccggaacatcaacgacacccgatacgcgagtaaagtgattctgaacagcctgcaaagtttcttcgggtctaaggaatgtgataccaaagtcaaagtggtacggggcactttcacgcaccaaatgagaatgaacttgaaaattgagaagaaccgggaagaaagttacgtccaccacgcagtcgacgcaatgctgattgccttcagccagatgggctacgacgcctaccacaagctcaccgagaaatacatagactacgagcacggagagttcgtggaccaaaagggatacgaaaagctgatcgagaacgacgtcgcctacagggaaacgacctaccagaacaaatggatgacaatcaagaagaacattgagatcgctgccgagaagaacaagtattggtatcaagtgaaccggaagtcaaacaggggactgtgtaatcaaaccatctacggcactcgtaaccttgacgggaaaaccgtgaaaatttctaagctcgacatccgcactgacgacggaatcaagaagttcaagggtattgttgagaagggcaagcttgagagattccttatgtaccgtaacgaccctaagaccttcgagtggctcctgcaaatctacaaagactactctgatagcaagaatcccttcgtgcagtacgagtccgaaacaggtgacgtgataaagaaggtaagcaagacaaacaacggccccaaagtctgcgagctgcgatacgaggacggggaagtgggaagttgcattgacatatcccacaaatacgggtacaagaaaggcagcaagaaagtgatcctggacagcctgaatccctatcgcatggacgtgtactacaataccaaagataacagatactacttcgtgggcgttaaatactctgatatcaaatgtcagggagactcttacgtgattgacgaagacaagtatgctgctgccctggtacaagagaagatcgtacctgaggggaaggggcgcagcgatctcactgaactgggctacgagttcaaactgtctttctacaagaacgaaattattgaatacgagaaggacggggagatctacgtcgagcgcttcctgtcaaggaccatgcccaaggtctccaactacatcgagacaaaaccccttgaggccgctaagttcgagaagcggaacctggtaggattggccaaaacatcaaggattcgaaagattagagtcgacattctcggcaacaggtatctgaactcaatggagaactttgacttcgtcgttggtcacaag

SEQ ID NO: 8 - DNA, 3315 bp, bacterium LF-3
  source 1..3315: mol_type = other DNA
atgagcagatatgtattaggattagatataggaattacttctgtagggtatggtgtaatagatattgataataatttatttgtggattatggtgtaaggcttttcaaagaaggaactgctgcagaaaatgaaacgcgaagaactaaaaggggttcaagacgtttaaaaagaagaaaatctaatcgtttaaatgatatgaaaaatcttttaaaggaaaatgacttatattttgaagattatcgaaattataatccttatgagataagggctaaaggattaaaagaaaagttattgcctgaagaactatgtacagcaattatgcatataacaaaatcaagaggaacaactttagaagcacttgctgatgaaagtcaagatgatgaaggaacaaaagctacactttcaaaaaatgctaaagaattaaatgatggaaaatatatttgtgaagttcaattggatagattaaataaggatcataaagtaagaggaacggaaaataatttcaaaacagaagattatgtcaaagaactcaaagaaatattaaaacaccaagatttaaatgaagaattgtgtgatcaaattattgaaatggtttcaagaagaagacgttatgatcaaggcccaggtagtgaaaaatcaccaactccttatggaagttatcgaatggtggatggtgttttaaaacatgttaatttgattgatgaaatgcgtggaagatgtagtgtctatccagatgaatttagagcgcctaaacaatcttatacagcagaattatttaatttgttaaatgatttaaataatttaacaattaaaggtgagaaaataacagttgaagaaaaagaaaaggttgttgcatttgttaatgaaaaaggaagtattacagtaaaacaattacttaaattattagatgctcaagaagatgaagttacaggatttagaattgataaaaatgataaaccattaattacagaatttaagggttatagtaaagttttaaaagtctttaaaaaatataaccaacaagaattactagaagataaattgattgttgatcaagttattgacatatgtacaaaatcaaaaggtattgatgaaagaaaaaaagatattaaagaattatatcctgaatttgataatgagttaattgaagaattagcttcagttaaaggtgtttctgcttatcattcattatcttttaaagcaatgcatataatcaataaagaaatgcttacaacagaaatgaatcaaatacaagttcttcatgaaatagaaatgtttgataaaaatagaaaatcattaaagggtaagaaaaatattgaacctgatgaagaagctattctatctccagttgctaaaagagcgcatcgagaaacatttaaagtcattaatgcgttaagaaaacaatatggcgaatttgatagtattgttattgaaatgacaagagataaaaattcaaaggaacaagtaaagcgaataaatgatagtcaaaaaagatttaaaagcgaaaatgatcgagttgatggaattattaaaaattcaggtattgatccagaaagagttaatggaaaaacaaaaacgaaaattcgtctttatttacaacaagattgtaagacggcctatacacaacaagatattgatttacatacattgatttttgatgataaagcttatgaaatagatcatattattccaatatctgtttcattggatgattctcttactaataaagtattagcttctcgtttagaaaaccaacaaaaaggtaatctaacaccaatgatggcttatttaaagggaaaatttacgggtggtaatttagaaaaatataaattatttgtaagtagtaataaaaattttaatggtaaaaaaagaaataatttacttactgaacaagatattacaaaagaagatgtagcaagaaagtttatcaatcgtaatttagtagatacaagctatgcttgtcgtacagtattaaatactttgcaacgctattttaaagataatgaaatagatacaaaagttcatactattagaggacaatcaaccaatatttttagaaaacgaataaatttacaaaaagatagagagcaagattattttcatcatgcaatcgatgcattgattgttgcttcgttaaagaaaatgaatattgtcaattcatatttaatgcattacaactatagtgatttatatgatgaagaaacaggggaagtatttgatgttttacctgataaacaatttattgatcaaagatatatttcatttatctctgatttaaaaaatatttatcaagaatcgaatcaatataacttaggttatattacccaagaacaaatgcattatccacttatcaaggtatctcataaaatagatacaaaaccaaataggaaaattgcggatgaaacaatatatagtacaagaaatattgaaggacaagatatgctagttgaaaaaataaaaaatatctatgatcctaaagaaaagaaagcaattgaacttgttaataatattattaatgatgatactgataagtacattatgaaacataaagatccacaaacttttgaaaaaataaaagaagtggtattaaatcattttaatgattataaagattcaaaagaatattatgtaattgacaaaaaaggtaagtattctttaaaagaagaaagtcctttaacatcatattataatgaaaatggagctattactaaatattctaagaaaaataatggaccagcaattacatcaatgaaattttactctgaaaaactaggaaatcatttagcaattacaagtaattataatacaaataataaaaaggtaattttaaaacaaataagcccatatcgaacagacttttatgtatctcctgaaggaaaatataaatttgttacagttagatataaagatgttttttataaagaaacaattcataaatttgtcatagatgaaaattggtatcatgaagaaaaaattaaaaaaggaattctagaagattggaaatttgtatgttcaatgcatcgagatgaacttattggacttatcaaacctgaaggtaaaaagtttgtttatgatgcttcaattaatggtggtcaaacacaatatcatgatggtaaacattatgaaatcttgaagtttacagcaacgaatgatgaaaagaaaagaacttttgaagtaaaaccgattaacactaactgctcaaaacgattaatgccatctgtaggaccttttattaaaattcaaaaatttgctacggatgttttaggaaatatatatgaagttaaagataatagattgaaattagagttcgattag

SEQ ID NO: 9 - DNA, 3309 bp, synthetic construct
  misc_feature 1..3309: note = Synthetic
  source 1..3309: mol_type = other DNA
tctaggtacgtgttgggactggacatcggcataacttccgtgggctacggggttatcgacatcgacaacaacctgttcgtcgactacggggtgagactgtttaaggaaggcacagccgcggagaacgagaccagacggaccaagagagggtcccgacgccttaagcgcaggaagagtaaccgccttaacgacatgaagaacctgctgaaagagaacgatctgtacttcgaggactacagaaactacaacccgtacgaaattcgagccaaggggttgaaggagaaacttctcccagaggagctgtgcaccgctatcatgcacatcactaagagtcgtgggactaccctggaagccttggccgacgagtctcaggacgacgagggcaccaaggccaccctcagcaagaacgcgaaggagcttaacgacggtaagtacatctgcgaggtgcagctggacaggttgaacaaagaccacaaggtccggggcactgagaacaactttaagaccgaggactacgttaaggaactgaaggagatcctcaagcatcaggacctgaacgaggagctctgcgaccagatcatcgagatggtatctcgtcgcaggcggtacgaccagggacccggctctgagaagtcccccacaccctacggttcttaccggatggtcgacggggtgttgaagcacgtgaacctgatcgacgagatgaggggccgatgctccgtgtacccggacgagttccgcgctccgaagcagagttacaccgctgagcttttcaacctgctgaacgacctcaacaacctcactatcaagggagaaaagattacggtcgaggagaaggagaaagtggtcgccttcgtgaacgagaaggggtctatcactgttaagcagcttctcaagctccttgacgcacaagaggacgaggtgaccggtttccgcatcgacaagaacgacaagcctctgatcaccgagttcaaaggatactcaaaggtgcttaaggtgttcaagaagtacaatcagcaggagcttctggaagacaagcttatcgtggaccaggtcatcgatatctgcactaagagcaagggcatcgacgagaggaagaaggacatcaaggagttgtacccagagttcgacaacgaactgatcgaggagttggcaagcgtcaagggcgtgtcagcataccacagtctgagcttcaaggctatgcacatcattaacaaggagatgctgaccaccgagatgaaccagattcaggtcctgcacgagatcgagatgttcgacaagaaccgcaagagcttgaaagggaagaagaacatcgagcccgacgaagaggccatcctgtcccccgtagccaagcgggcacaccgcgagaccttcaaggtgatcaacgcccttcgtaagcagtacggggagttcgactcaatcgtgatcgagatgacccgcgacaagaactccaaagagcaggtgaaacggatcaacgactctcagaagcgtttcaagtcagagaacgacagagtggacggtatcatcaagaactctggaatagaccccgagcgtgtcaacggcaagaccaagacaaagatacgcctctacctgcagcaggactgcaaaactgcgtacacccagcaggacatcgacctgcacactcttatattcgacgacaaggcgtacgagatcgaccacataatccctatcagcgtcagtcttgacgacagtctgaccaacaaggttctggcctcaaggctcgagaatcagcagaaggggaacctcacccctatgatggcctacctcaaaggtaagttcactggcggaaacctggagaagtacaagctgttcgtgtcatccaacaagaacttcaacggcaagaagcgcaacaacctgctgaccgagcaggacataactaaggaagacgtggctcgaaaattcattaacagaaacctggtggacacatcctacgcctgcagaaccgtcttgaacacactgcagaggtacttcaaggacaacgagattgacactaaggtacacacaatccgaggccagagcacaaacatcttccgcaagcgcattaacctgcagaaggaccgcgaacaggactacttccaccacgccattgacgccctgatcgtggccagtctgaagaagatgaacatcgtgaacagctacctgatgcactataattacagcgacttgtacgacgaggagactggcgaggtcttcgacgtgctgcccgacaagcagttcatcgaccagcggtacatctccttcatttccgacctgaagaacatctaccaggagtccaaccagtacaatctgggatacataactcaggagcagatgcactacccgctgattaaagtcagccacaagattgacaccaagcccaaccggaagatagctgacgagactatctacagcacccgcaacatcgagggccaggacatgttggtggagaagattaagaacatttacgaccccaaggagaagaaggccatcgagctggtgaacaacataatcaacgacgacaccgacaaatatatcatgaagcacaaggacccccagaccttcgagaagatcaaagaggtcgtcctgaaccacttcaacgactacaaggactctaaagagtactacgtcatcgataagaaggggaaatacagcctgaaagaggagagccccctgactagctactacaacgagaacggggccataacgaagtacagcaagaagaacaacgggcccgctataacatccatgaagttctatagcgagaagctcggcaaccacctggctatcactagcaactacaacacgaacaacaagaaagtgatcctcaagcagatttcaccctaccgtactgatttctacgtgagtccagagggcaagtacaagttcgtgaccgtccggtacaaggacgtgttctacaaggagaccatccacaagttcgttatcgacgagaactggtatcacgaggagaagataaagaagggtatcctggaagactggaagttcgtttgctctatgcaccgggacgagctgatcgggctgattaagccagagggcaagaaattcgtgtacgacgcgtccatcaacggcggacagactcagtaccacgacggcaagcactacgagattcttaaattcaccgccaccaacgacgagaagaagaggaccttcgaggtgaagcccatcaatacaaattgtagtaagaggttgatgccttccgtcggccccttcatcaagatccagaagttcgccaccgacgtcctggggaacatctacgaggtgaaggacaacaggcttaagctggaatttgac

SEQ ID NO: 10 - DNA, 3312 bp, synthetic construct
  misc_feature 1..3312: note = Synthetic
  source 1..3312: mol_type = other DNA
atgtctaggtacgtgttgggactggacatcggcataacttccgtgggctacggggttatcgacatcgacaacaacctgttcgtcgactacggggtgagactgtttaaggaaggcacagccgcggagaacgagaccagacggaccaagagagggtcccgacgccttaagcgcaggaagagtaaccgccttaacgacatgaagaacctgctgaaagagaacgatctgtacttcgaggactacagaaactacaacccgtacgaaattcgagccaaggggttgaaggagaaacttctcccagaggagctgtgcaccgctatcatgcacatcactaagagtcgtgggactaccctggaagccttggccgacgagtctcaggacgacgagggcaccaaggccaccctcagcaagaacgcgaaggagcttaacgacggtaagtacatctgcgaggtgcagctggacaggttgaacaaagaccacaaggtccggggcactgagaacaactttaagaccgaggactacgttaaggaactgaaggagatcctcaagcatcaggacctgaacgaggagctctgcgaccagatcatcgagatggtatctcgtcgcaggcggtacgaccagggacccggctctgagaagtcccccacaccctacggttcttaccggatggtcgacggggtgttgaagcacgtgaacctgatcgacgagatgaggggccgatgctccgtgtacccggacgagttccgcgctccgaagcagagttacaccgctgagcttttcaacctgctgaacgacctcaacaacctcactatcaagggagaaaagattacggtcgaggagaaggagaaagtggtcgccttcgtgaacgagaaggggtctatcactgttaagcagcttctcaagctccttgacgcacaagaggacgaggtgaccggtttccgcatcgacaagaacgacaagcctctgatcaccgagttcaaaggatactcaaaggtgcttaaggtgttcaagaagtacaatcagcaggagcttctggaagacaagcttatcgtggaccaggtcatcgatatctgcactaagagcaagggcatcgacgagaggaagaaggacatcaaggagttgtacccagagttcgacaacgaactgatcgaggagttggcaagcgtcaagggcgtgtcagcataccacagtctgagcttcaaggctatgcacatcattaacaaggagatgctgaccaccgagatgaaccagattcaggtcctgcacgagatcgagatgttcgacaagaaccgcaagagcttgaaagggaagaagaacatcgagcccgacgaagaggccatcctgtcccccgtagccaagcgggcacaccgcgagaccttcaaggtgatcaacgcccttcgtaagcagtacggggagttcgactcaatcgtgatcgagatgacccgcgacaagaactccaaagagcaggtgaaacggatcaacgactctcagaagcgtttcaagtcagagaacgacagagtggacggtatcatcaagaactctggaatagaccccgagcgtgtcaacggcaagaccaagacaaagatacgcctctacctgcagcaggactgcaaaactgcgtacacccagcaggacatcgacctgcacactcttatattcgacgacaaggcgtacgagatcgaccacataatccctatcagcgtcagtcttgacgacagtctgaccaacaaggttctggcctcaaggctcgagaatcagcagaaggggaacctcacccctatgatggcctacctcaaaggtaagttcactggcggaaacctggagaagtacaagctgttcgtgtcatccaacaagaacttcaacggcaagaagcgcaacaacctgctgaccgagcaggacataactaaggaagacgtggctcgaaaattcattaacagaaacctggtggacacatcctacgcctgcagaaccgtcttgaacacactgcagaggtacttcaaggacaacgagattgacactaaggtacacacaatccgaggccagagcacaaacatcttccgcaagcgcattaacctgcagaaggaccgcgaacaggactacttccaccacgccattgacgccctgatcgtggccagtctgaagaagatgaacatcgtgaacagctacctgatgcactataattacagcgacttgtacgacgaggagactggcgaggtcttcgacgtgctgcccgacaagcagttcatcgaccagcggtacatctccttcatttccgacctgaagaacatctaccaggagtccaaccagtacaatctgggatacataactcaggagcagatgcactacccgctgattaaagtcagccacaagattgacaccaagcccaaccggaagatagctgacgagactatctacagcacccgcaacatcgagggccaggacatgttggtggagaagattaagaacatttacgaccccaaggagaagaaggccatcgagctggtgaacaacataatcaacgacgacaccgacaaatatatcatgaagcacaaggacccccagaccttcgagaagatcaaagaggtcgtcctgaaccacttcaacgactacaaggactctaaagagtactacgtcatcgataagaaggggaaatacagcctgaaagaggagagccccctgactagctactacaacgagaacggggccataacgaagtacagcaagaagaacaacgggcccgctataacatccatgaagttctatagcgagaagctcggcaaccacctggctatcactagcaactacaacacgaacaacaagaaagtgatcctcaagcagatttcaccctaccgtactgatttctacgtgagtccagagggcaagtacaagttcgtgaccgtccggtacaaggacgtgttctacaaggagaccatccacaagttcgttatcgacgagaactggtatcacgaggagaagataaagaagggtatcctggaagactggaagttcgtttgctctatgcaccgggacgagctgatcgggctgattaagccagagggcaagaaattcgtgtacgacgcgtccatcaacggcggacagactcagtaccacgacggcaagcactacgagattcttaaattcaccgccaccaacgacgagaagaagaggaccttcgaggtgaagcccatcaatacaaattgtagtaagaggttgatgccttccgtcggccccttcatcaagatccagaagttcgccaccgacgtcctggggaacatctacgaggtgaaggacaacaggcttaagctggaatttgac

SEQ ID NO: 11 - DNA, 4113 bp, Ezakiella peruensis strain M6.X2
  source 1..4113: mol_type = other DNA
atgacaaaagtaaaagattattatatcggacttgatataggtacatcatcagttggctgggcagtaacagacgaggcttacaatgttctaaaattcaactccaagaagatgtggggagttcgtctttttgatgatgccaaaactgctgaagaaagacgagggcaaagaggggccaggagaagacttgaccgcaaaaaagaacgcttaagtctcttgcaagatttttttgcagaggaagttgctaaagtagatccaaatttctttttgcgtctagataacagcgacctttatatggaggacaaagatcaaaagttaaagtccaagtacactttatttaatgataaagattttaaagacaagaacttccacaaaaaatatccgactatccaccatctccttatggacttgattgaagatgatagcaaaaaagatattagactggtttatttagcttgccattacttacttaaaaatcgtggccactttatttttgaaggacaaaaatttgatacaaagagctcctttgaaaattctctaaatgaattaaaggtccacttaaatgatgaatacggtcttgatcttgagtttgataatgaaaatttgataaatatacttacagatcctaagttaaacaagaccgcaaaaaagaaagaacttaaaagtgttattggagatacaaaatttctaaaggcagtatctgctattatgattggtagctctcaaaagctagtagatctatttgaaaatcctgaagactttgatgattcggcaatcaaatcagtggatttttctacgacgagttttgatgataaatatagcgattacgagttagcccttggggataaaattgcccttgtaaatatattaaaagaaatctatgactcatctatacttgaaaatttattaaaagaagccgataaatcaaaagatggcaataagtacatttctaacgcctttgtaaaaaaatataacaagcatggccaggacctcaaggaatttaagcgcctagttagacagtaccataaatcagcctacttcgacatctttaggagtgaaaaagtaaacgataactatgtttcatataccaagtcaagtatatccaataacaagagagtgaaggcgaataagtttacagaccaagaagctttttataagtttgctaaaaagcacctagaaactataaaatacaaaattaataaagttaatggtagcaaagctgaccttgaactaatagatggaatgctaagggatatggaatttaaaaatttcatgccaaagataaaatcttctgataatggagttataccttatcaattgaaacttatggagctaaataagatccttgaaaaccaatccaaacaccatgaatttttaaacgtatccgatgaatatggaagcgtttgcgacaagattgcttcgattatggaatttaggattccatattatgttgggcctttaaatcctaactcaaaatatgcttggattaagaagcaaaaggacagcgaaatcacgccatggaattttaaagatgtagttgatttggattcttcaagggaagagtttatagatagcttaattggcaggtgcacatatttaaaagatgaaaaagttctaccaaaggcctcgcttctctacaatgagtatatggttttaaatgaactcaacaatttaaaattaaatgatcttcctattactgaagaaatgaagaagaaaatcttcgatcaactctttaagaccaggaaaaaagtaacattaaaggctgtcgctaatcttctcaaaaaagaatttaatataaatggagaaatcctattgtccggcacagatggggattttaaacaagggctaaactcttataacgattttaaggccattgttggggacaaggttgacagcgacgactatagggataaaatcgaagaaattatcaagctaatcgtcctctatggagatgacaaatcttacttgcaaaagaaaataaaggcgggatacggcaagtattttacagattcagaaatcaaaaagatggctggcctaaattataaagactggggcagattaagtaaaaaactactcacaggtttagaaggcgccaataaaattacaggcgaaagaggatctataatccattttatgcgtgagtacaatttaaacttaatggaattaatgagcgccagcttcacttttacagaggaaattcaaaagttaaatccagttgacgatagaaaactctcctatgagatggttgatgagctttatttatcaccttcagttaagagaatgttatggcaaagtctaagaatagttgatgaaattaaaaatataatgggcactgattccaagaaaatctttattgaaatggccaggggcaaagaagaagtcaaggctagaaaagaatctagaaaaaatcagctcttaaaattttacaaggatggcaaaaaagcctttatatcagaaatcggcgaagaaagatatagctatcttttaagtgaaatcgaaggagaagaggaaaacaaattcagatgggacaatctttatctctactacacccagcttggcaggtgtatgtatagtcttgagccaattgatatttcagaactctcatcgaaaaacatctatgaccaagaccacatttatccaaagtcaaaaatctatgatgattcaattgaaaacagagttttggttaagaaagatttaaatagcaagaaaggcaattcatacccaataccggatgagattttaaataaaaattgctatgcttattggaaaattctatatgacaagggactaattggtcaaaagaaatataccagacttacacgtaggacaggatttactgatgatgaacttgtccaatttatatccaggcaaatagttgagaccaggcaggctaccaaagaaacagcaaatctcttaaaaaccatttgcaaaaattcagaaatagtttactctaaggcagaaaatgctagcagattcagacaggaatttgatatagtaaaatgccgtgcagtcaatgacctccaccacatgcatgacgcttatataaatataatcgttggcaatgtctacaatacaaaatttaccaaagaccccatgaactttgtcaaaaaacaagagaaagctagaagttataacttggaaaacatgtttaaatatgacgtaaagcgcgggggctatacagcatggatagcagacgatgaaaaaggcactgttaaaaatgctagcatcaagagaataagaaaagaactagaggggaccaactacagatttactcgcatgaattatatagaaagtggtgcactatttaatgctaccctgcaaagaaaaaacaaaggaagtcgccctctaaaagataaggggcctaagagctcaatagaaaaatatggtggatatactaatataaacaaggcttgctttgcagtgttggatattaaatcaaaaaataaaatagaaagaaaattaatgccagttgaaagagaaatatacgctaagcaaaagaatgataaaaaattgagtgatgaaatatttagcaaatatttgaaagatagattcggaattgaagattatagagtagtatatcctgtagtaaagatgagaactttgttaaaaatagatggatcttattattttataactggtggaagtgacaaaacattggaattaaggagtgcacttcaattaatattaccaaagaaaaatgaatgggcaataaagcaaattgataaatccagtgagaatgattacctaacaattgaaaggatacaagatttaacggaagaacttgtatacaatacgtttgatataatagtgaataaatttaaaacatctgtatttaaaaaatcatttttgaatttattccaagatgataaaatcgaaaatatagattttaaattcaaatcaatggattttaaagaaaagtgtaaaactctattgatgctagtaaaagccatcagagcttctggtgtacgccaagacttaaaatctatagatttaaaatcagactatggtagattgagctccaagactaataatataggaaactatcaagaatttaaaatcataaaccaatcaattacaggcctctttgaaaacgaagtggacttgttaaaattatga

SEQ ID NO: 12 - DNA, 4107 bp, synthetic construct
  misc_feature 1..4107: note = Synthetic
  source 1..4107: mol_type = other DNA
accaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgagaacctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggaccttgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgcttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctg

SEQ ID NO: 13 - DNA, 4110 bp, synthetic construct
  misc_feature 1..4110: note = Synthetic
  source 1..4110: mol_type = other DNA
atgaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgagaacctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggaccttgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgcttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctg

SEQ ID NO: 14 - DNA, 4110 bp, Clostridium sp. AF02-29
  source 1..4110: mol_type = other DNA
atgaaagagaaaatggaatactatttaggtcttgacatgggaaccaattcagtcggatgggctgtaacagataaagaatatcgtttgatgcgggcgaagggaaaagatttgtggggagttcgtttgttcgaacgtgctaatacagctgaagaacggcgggcatataggattaaccgaagaagacgtcagcgggaagtagctagaattggaattttaaaagaattatttgccgatgaaattgcaaaagttgatgctaatttttttgcacgcctggacgacagtaaatattatcttgatgatagacaggaaaataataaacagaagtatgcgatatttgctgataaagattacacggacaaagagtattttagccaatatcagacaatttttcatctgagaaaagaactgatcctgtcagatcaacctcatgatgttcgtcttatttatctggcgctgttaaatatgtttaaacatagaggccattttttaaataaaacgttgggaacctcggaatcattagaatcgttttttgatatgtatcaaagattagctgtatgtgcggatggagagggaatcaaacttccagaaacggtggatttaaagaaattagaacagatacttggagcacgtggatgctctagaaaggcaacattggaacatatatctgaaataatggggattaataaaaagaataaaccagtttatagcctcatgcagatgatatgtggacttgatactaaaatgatagacctttttgggcagaagattgatgaagaacacaaaaaaatctctctttcatttcgaacgtccaattatgaagaaatggcagaggaagtccgtaatacgataggggatgatgcatttgaacttatattgacagcaaaagaaatgcatgattttggcttgctggcggagattatgaaaggatattcatatttgtcagaagcgcgggtggctgtctatgaagagcatcgaaaggatttggctaaactgaaagccgtctttaaacaatatgaccataaggcatatgatgaaatgtttcgaatcatgaagaatggtacttatagtgcctatgttggaagtgtaaatagtttcggtaaaatagagagaaggacagtaaaaacttccagagaagaattgttaaaaaatataaagaaaattttaacagggtttccagaggatgatgctacagtacaggaatttttgggtaagatagattcggatacacttctccaaaaacaactgacagcttctaatggagtgattccaaatcaggtacatgcaaaagaaatgaaggtcattttgaaaaatgcagaaaaataccttccatttttaagtgaaagagatgaaacaggattaagtgtatcagaaaaaataatagctctgtttacatttacgatcccgtactatgttggtcctcttgggcagcaacatttaggaaaggaatgtgcacatggctgggtagagcgaaaagaaaaaggtactgtgtatccatggaattttgaacaaaaggttgatttaaaggcaagtgcagaacattttatagaaagaatggtaaaacattgcacgtatttatctgatgagcaggcattgccaaaacaatcattgttgtatgaaaaatttcaggtattgaatgaattaaacaatttaaaaattcgaggagaaaaaatatcggtagaattaaaacagcagatatatcgggatgtctttgaacatactgggaaaaaagtatcgatgaagcagttggaaaactatctgaagttgaacggcctgcttgaaaaagacgaaaaggacgcagttacaggaatagatggtggtttccatagttatttgtcctctttaggaaaatttataggaattttgggagaagaagctcattatggtaaaaaccagaatatgatggaaaaaattgtattttgggggacagtatatggacaggataaaaaattccttcgcgaacggttaagtgaagtttatggagatagattgtcaaaagagcagattcgtcgtattactggtatgaaatttgaaggatggggacgactttctaaagaatttcttttactggagggggcttctagagaagaaggggagattcggacattgattcgttcattatgggagacaaatgaaaatttgatggggcttttaagtgaacgatatacatatagcgaagaagtacgagaaaaaacgctagagtgtgagaagagcctttctgaatggacgattgaagatttggaaggaatgtatctgtcagcaccggttaagcgcatggtatggcagactttgttaattgtaaaagagcttgaaaaggtgctgggatgtgctccacgacgtatttttgtggagatggcacgcgaagatgcggagaaaggaaggagaacagaatcacgaaagcagaaattgcagaatctttataaagcaattaaaaaagaggagatagactggaaaaaagagattgatgaaaaaacagagcaggcattccgcagtaaaaaattatatttgtattacctgcagaaggggcgctgtatgtatacgggcgagtctattcgatttgaagatttgatgaatgataatttatatgatatcgatcatatttatccgagacattttgtgaaggatgatagtttagagcagaatctggtgctggtaaagaaggaaaaaaatgcacataaaagtgatgtatttccgattgaggcggatattcagaaaaagatgagtccgttctggaaagaactgaaagaaagaggttttatatcagaagaaaagtatatgcgtttaacgaggaggtatggcttttcggaagaggaaaaagcaggttttatcaatcggcaattggtggaaacaagacagggaacaaagagtattacagagatattgggacaagcttttccagatgtggatatcatattttcaaaagcgtcgaatgtgtcggagttcagacatatttatggattgtataaggttcgcagtataaatgattttcatcatgcacatgacgcatacttaaatatagtggttggaaatacgtatcatgtgaaatttacgaaaaatccgttgaattttattcgggaagcagaaaaaaatccgcagaatgcagaaaataaatacaatatgaaccggatgttcgattggacagtaaaaagaggaaatgaaacggcatggatagcaagttccgataaagaagcaggtagtattaaaattgtaaaggctatattggcaaaaaatacaccattagttactaagaggtgtgcggaggcacatggaggaataaccaggaaagcaactatctggaataaaaacaaagctgctggcagcggatatattccggttaagatgaatgatgcaagacttttggatgttacaaaatatggcggtttgacatctgtatcagcttcggggtacactttgctggaatatgatgtaaaaggtaaaaaaataagaagtcttgaggcgattccaatttacttgggacgagtgtctgaattgacaaatgaggcgattttgaaatattttgaaaaagtgctgatagaggaaaataagggaaaagaaataacagaacttcgcatttgtaagaagtttattccgagagagtcgttagtgagatataatgggtattactattatctcggaggaaaatcagtggaacagattgtattgaaaaatgcgacacagatggcatattcagaagaagaaacatgttatataaaaaagatagaaaaagctatagaaaaaacatattatgaagaagtggacaaaaataaaaatgtgattttgacaaaaactagaaacaatgcaatgtatgataaatttattataaaatatcagaattcaatttatcagaatcaaagtggtgcaatgaaaaattctattattggaaaaagaaatgaatttttaacattatcattggaaaagcagtgtagaatattgaaggcactagtagaatattttaggacaggagatattattgatttgagagaattaggaggtagttcacaggcaggaaaagtggctatgaacaagaaaattatgggagcaagtgaattagtgctaataagtcaatctccaacaggtttattccaacaagagattgatttactaaaaatatga

SEQ ID NO: 15 - DNA, 4104 bp, synthetic construct
  misc_feature 1..4104: note = Synthetic
  source 1..4104: mol_type = other DNA
aaggaaaagatggagtattacctggggctggatatgggcactaacagcgtgggttgggcggtgaccgacaaggagtaccggctgatgagggcaaaagggaaggacctgtggggcgtacggctgtttgagagagcgaacactgcggaagagaggcgcgcctacagaatcaatagacgacggcggcaacgagaggttgcaaggatcggtatccttaaggaactcttcgctgacgagatcgccaaggtggacgcaaacttcttcgccagacttgatgattcaaagtactacctggacgaccggcaagagaacaacaagcagaaatacgctattttcgccgacaaggactatactgataaggaatacttctcccagtaccaaactatcttccatctccggaaggagcttatactcagtgaccagccacacgacgtgagactgatctaccttgctcttctgaacatgttcaagcaccggggacacttcttgaacaagactctggggacttccgagagtttggagtctttcttcgacatgtaccagcgactggcagtgtgcgcagacggggaaggcattaagttgcccgagaccgtagaccttaagaagctcgagcaaatcctgggcgcccggggatgtagcaggaaagccacccttgagcacatcagcgagattatgggaatcaacaagaagaacaagcccgtctactccctgatgcaaatgatttgcggtctggacaccaagatgatcgatctgttcggacaaaagatcgacgaggagcataagaagataagcctgtccttcagaactagcaactacgaggagatggccgaagaggttagaaacacaattggcgacgacgccttcgagctgattctcactgccaaggagatgcacgacttcgggctgttggctgaaatcatgaaggggtactcctacctgagcgaggctcgcgttgccgtgtacgaggaacaccggaaagacctggccaagctcaaggcagtgttcaagcagtacgatcacaaagcttacgacgagatgttcaggattatgaagaacgggacatactcagcttacgtagggtccgtgaactcctttggcaagatcgaacgcagaaccgtgaagacctctcgcgaggagcttcttaagaacattaagaagatcctgaccggtttccccgaagacgacgcaactgtgcaagagttcctcgggaaaattgactctgacacgctgcttcagaagcagttgactgccagcaacggcgtaatccctaaccaagtccacgcgaaggagatgaaagtaatcctgaagaacgccgagaagtatctgcctttcctgtccgagagggacgagactgggctctcagtctccgagaagatcattgcattgttcacgttcactattccttattacgtgggacccctgggtcaacagcacttggggaaagagtgcgcccacggttgggtggaaagaaaggagaaggggaccgtttacccctggaacttcgagcagaaagtcgaccttaaagcttccgctgagcacttcattgagcgcatggtgaagcactgtacatacctgtccgacgaacaagctctgcccaagcagagtctgctctacgagaagttccaagtgcttaacgagctcaataacttgaagatcaggggcgagaagatcagtgtggagctgaagcaacaaatttaccgggacgttttcgagcacaccggaaagaaggtttcaatgaaacaactggagaattacttgaaactgaatgggcttctggagaaggatgagaaagatgccgtgaccgggatcgacggcggatttcactcatacctttcttccctgggcaagttcatcggcatcctcggggaagaggcacactacggaaagaatcaaaacatgatggagaagatcgtgttctggggtacggtgtacgggcaagacaagaagtttctgcgggagcgtctgtccgaggtgtacggcgaccggctgagcaaggaacaaatcagaaggataacaggaatgaagttcgagggctggggccggctctccaaggagttcctgctgctcgaaggagcaagtcgggaagagggcgaaatccgcaccctcatacggagcctgtgggaaacgaacgagaacctgatgggactgctgtcagagagatacacttactcagaagaggtccgcgagaagactctcgaatgcgagaaatctctgtcagagtggaccatcgaggacctcgagggcatgtacctttccgcccctgtaaaacggatggtctggcaaaccctcttgatagtgaaggaactggagaaagtcctcggctgcgcccctcgaaggatcttcgttgaaatggctagagaggacgcagaaaagggtcgccggaccgagtcccgcaaacaaaagctgcaaaacctgtacaaggctatcaagaaggaagaaattgattggaagaaggaaatcgacgagaagaccgaacaagcctttaggagcaagaagctgtacctgtactatctccagaaaggacgatgcatgtacaccggagaaagcatccgcttcgaggacctcatgaacgacaacttgtacgacatagaccacatctacccccggcacttcgttaaagacgactcccttgaacaaaacctcgttttggttaagaaggagaagaacgctcacaagagcgacgtgttcccaatcgaagccgacatacaaaagaaaatgtctcccttttggaaggagctcaaggagaggggattcatctctgaggagaaatacatgagactcactcgaagatacgggttcagtgaggaagagaaggctggattcattaacagacagctggtagagacccgtcaaggcacgaaatctatcactgaaatcctgggccaggccttccccgacgttgacataattttctccaaggcttcaaacgtttcagaatttcggcacatctacggcctctacaaagtgaggtctattaacgacttccaccacgcgcacgatgcttatctgaacatcgtcgtaggcaacacttaccacgttaagttcacaaagaaccccctgaacttcatccgcgaggccgagaagaacccacaaaacgccgagaacaagtataacatgaatcgcatgtttgactggaccgtgaagaggggcaacgagactgcctggatcgccagcagtgacaaagaggccggatctatcaagatagtcaaagcgattcttgccaagaacacccctcttgtgaccaaacggtgcgcagaagctcacggcggcattactcgcaaggcgacaatttggaacaagaataaggccgcgggttctggctacatcccagtgaaaatgaacgacgcccggctcctggacgtgaccaagtacggcggactgacctcagtgagtgcgtccggctataccctgcttgagtacgacgtgaaggggaagaagattcgatccctggaagctatccccatctatcttgggagagtcagtgagctcactaacgaagccatcctcaagtacttcgagaaggttcttatcgaagagaacaaagggaaggagattaccgagctccgtatctgcaagaagttcataccccgtgaaagcctcgttcggtacaacggatactattactacctgggcggcaagtctgttgagcaaatagtcctgaagaacgccacccaaatggcttactccgaggaagagacttgctacatcaagaaaattgagaaggcaattgagaagacctactacgaagaggtcgataagaacaagaacgtaatactgactaagacccgcaataacgcgatgtacgacaagttcatcattaagtaccaaaacagtatataccaaaaccagagcggagccatgaagaactcaatcatagggaagaggaacgagttcctgactctcagtctcgagaaacaatgccgcatcctcaaagctctggtcgagtacttccggaccggggacatcatagacctgcgggagctcggcggatcaagccaagcgggcaaggtcgcgatgaataagaagatcatgggcgcgagcgagctggtcctgatttcacagtcccccaccgggttgtttcagcaggaaatcgacctgctgaagatt

SEQ ID NO: 16 - DNA, 4107 bp, synthetic construct
  misc_feature 1..4107: note = Synthetic
  source 1..4107: mol_type = other DNA
atgaaggaaaagatggagtattacctggggctggatatgggcactaacagcgtgggttgggcggtgaccgacaaggagtaccggctgatgagggcaaaagggaaggacctgtggggcgtacggctgtttgagagagcgaacactgcggaagagaggcgcgcctacagaatcaatagacgacggcggcaacgagaggttgcaaggatcggtatccttaaggaactcttcgctgacgagatcgccaaggtggacgcaaacttcttcgccagacttgatgattcaaagtactacctggacgaccggcaagagaacaacaagcagaaatacgctattttcgccgacaaggactatactgataaggaatacttctcccagtaccaaactatcttccatctccggaaggagcttatactcagtgaccagccacacgacgtgagactgatctaccttgctcttctgaacatgttcaagcaccggggacacttcttgaacaagactctggggacttccgagagtttggagtctttcttcgacatgtaccagcgactggcagtgtgcgcagacggggaaggcattaagttgcccgagaccgtagaccttaagaagctcgagcaaatcctgggcgcccggggatgtagcaggaaagccacccttgagcacatcagcgagattatgggaatcaacaagaagaacaagcccgtctactccctgatgcaaatgatttgcggtctggacaccaagatgatcgatctgttcggacaaaagatcgacgaggagcataagaagataagcctgtccttcagaactagcaactacgaggagatggccgaagaggttagaaacacaattggcgacgacgccttcgagctgattctcactgccaaggagatgcacgacttcgggctgttggctgaaatcatgaaggggtactcctacctgagcgaggctcgcgttgccgtgtacgaggaacaccggaaagacctggccaagctcaaggcagtgttcaagcagtacgatcacaaagcttacgacgagatgttcaggattatgaagaacgggacatactcagcttacgtagggtccgtgaactcctttggcaagatcgaacgcagaaccgtgaagacctctcgcgaggagcttcttaagaacattaagaagatcctgaccggtttccccgaagacgacgcaactgtgcaagagttcctcgggaaaattgactctgacacgctgcttcagaagcagttgactgccagcaacggcgtaatccctaaccaagtccacgcgaaggagatgaaagtaatcctgaagaacgccgagaagtatctgcctttcctgtccgagagggacgagactgggctctcagtctccgagaagatcattgcattgttcacgttcactattccttattacgtgggacccctgggtcaacagcacttggggaaagagtgcgcccacggttgggtggaaagaaaggagaaggggaccgtttacccctggaacttcgagcagaaagtcgaccttaaagcttccgctgagcacttcattgagcgcatggtgaagcactgtacatacctgtccgacgaacaagctctgcccaagcagagtctgctctacgagaagttccaagtgcttaacgagctcaataacttgaagatcaggggcgagaagatcagtgtggagctgaagcaacaaatttaccgggacgttttcgagcacaccggaaagaaggtttcaatgaaacaactggagaattacttgaaactgaatgggcttctggagaaggatgagaaagatgccgtgaccgggatcgacggcggatttcactcatacctttcttccctgggcaagttcatcggcatcctcggggaagaggcacactacggaaagaatcaaaacatgatggagaagatcgtgttctggggtacggtgtacgggcaagacaagaagtttctgcgggagcgtctgtccgaggtgtacggcgaccggctgagcaaggaacaaatcagaaggataacaggaatgaagttcgagggctggggccggctctccaaggagttcctgctgctcgaaggagcaagtcgggaagagggcgaaatccgcaccctcatacggagcctgtgggaaacgaacgagaacctgatgggactgctgtcagagagatacacttactcagaagaggtccgcgagaagactctcgaatgcgagaaatctctgtcagagtggaccatcgaggacctcgagggcatgtacctttccgcccctgtaaaacggatggtctggcaaaccctcttgatagtgaaggaactggagaaagtcctcggctgcgcccctcgaaggatcttcgttgaaatggctagagaggacgcagaaaagggtcgccggaccgagtcccgcaaacaaaagctgcaaaacctgtacaaggctatcaagaaggaagaaattgattggaagaaggaaatcgacgagaagaccgaacaagcctttaggagcaagaagctgtacctgtactatctccagaaaggacgatgcatgtacaccggagaaagcatccgcttcgaggacctcatgaacgacaacttgtacgacatagaccacatctacccccggcacttcgttaaagacgactcccttgaacaaaacctcgttttggttaagaaggagaagaacgctcacaagagcgacgtgttcccaatcgaagccgacatacaaaagaaaatgtctcccttttggaaggagctcaaggagaggggattcatctctgaggagaaatacatgagactcactcgaagatacgggttcagtgaggaagagaaggctggattcattaacagacagctggtagagacccgtcaaggcacgaaatctatcactgaaatcctgggccaggccttccccgacgttgacataattttctccaaggcttcaaacgtttcagaatttcggcacatctacggcctctacaaagtgaggtctattaacgacttccaccacgcgcacgatgcttatctgaacatcgtcgtaggcaacacttaccacgttaagttcacaaagaaccccctgaacttcatccgcgaggccgagaagaacccacaaaacgccgagaacaagtataacatgaatcgcatgtttgactggaccgtgaagaggggcaacgagactgcctggatcgccagcagtgacaaagaggccggatctatcaagatagtcaaagcgattcttgccaagaacacccctcttgtgaccaaacggtgcgcagaagctcacggcggcattactcgcaaggcgacaatttggaacaagaataaggccgcgggttctggctacatcccagtgaaaatgaacgacgcccggctcctggacgtgaccaagtacggcggactgacctcagtgagtgcgtccggctataccctgcttgagtacgacgtgaaggggaagaagattcgatccctggaagctatccccatctatcttgggagagtcagtgagctcactaacgaagccatcctcaagtacttcgagaaggttcttatcgaagagaacaaagggaaggagattaccgagctccgtatctgcaagaagttcataccccgtgaaagcctcgttcggtacaacggatactattactacctgggcggcaagtctgttgagcaaatagtcctgaagaacgccacccaaatggcttactccgaggaagagacttgctacatcaagaaaattgagaaggcaattgagaagacctactacgaagaggtcgataagaacaagaacgtaatactgactaagacccgcaataacgcgatgtacgacaagttcatcattaagtaccaaaacagtatataccaaaaccagagcggagccatgaagaactcaatcatagggaagaggaacgagttcctgactctcagtctcgagaaacaatgccgcatcctcaaagctctggtcgagtacttccggaccggggacatcatagacctgcgggagctcggcggatcaagccaagcgggcaaggtcgcgatgaataagaagatcatgggcgcgagcgagctggtcctgatttcacagtcccccaccgggttgtttcagcaggaaatcgacctgctgaagatt

SEQ ID NO: 17 - RNA, 16 nt, synthetic construct
  misc_feature 1..16: note = Synthetic
  source 1..16: mol_type = other RNA
gttttagtacctagag

SEQ ID NO: 18 - RNA, 18 nt, synthetic construct
  misc_feature 1..18: note = Synthetic
  source 1..18: mol_type = other RNA
ctttagacctactaaaat

SEQ ID NO: 19 - RNA, 19 nt, synthetic construct
  misc_feature 1..19: note = Synthetic
  source 1..19: mol_type = other RNA
gttttagtacctagagaaa

SEQ ID NO: 20 - RNA, 21 nt, synthetic construct
  misc_feature 1..21: note = Synthetic
  source 1..21: mol_type = other RNA
tttctttagacctactaaaat

SEQ ID NO: 21 - RNA, 46 nt, synthetic construct
  misc_feature 1..46: note = Synthetic
  source 1..46: mol_type = other RNA
aaggctttatgccgagattaaaggatgccgacgggcatcctttttt

SEQ ID NO: 22 - RNA, 11 nt, synthetic construct
  misc_feature 1..11: note = Synthetic
  source 1..11: mol_type = other RNA
ggctttatgcc

SEQ ID NO: 23 - RNA, 23 nt, synthetic construct
  misc_feature 1..23: note = Synthetic
  source 1..23: mol_type = other RNA
aaggatgccgacgggcatccttt

SEQ ID NO: 24 - RNA, 84 nt, synthetic construct
  misc_feature 1..84: note = Synthetic
  source 1..84: mol_type = other RNA
gttttagtacctagaggaaactttagacctactaaaataaggctttatgccgagattaaaggatgccgacgggcatcctttttt

SEQ ID NO: 25 - RNA, 90 nt, synthetic construct
  misc_feature 1..90: note = Synthetic
  source 1..90: mol_type = other RNA
gttttagtacctagagaaagaaatttctttagacctactaaaataaggctttatgccgagattaaaggatgccgacgggcatcctttttt

SEQ ID NO: 26 - RNA, 90 nt, synthetic construct
  misc_feature 1..90: note = Synthetic
  source 1..90: mol_type = other RNA
gtttaagtacctagagaaagaaatttctttagacctacttaaataaggctttatgccgagattaaaggatgccgacgggcatcctttttt

SEQ ID NO: 27 - RNA, 16 nt, synthetic construct
  misc_feature 1..16: note = Synthetic
  source 1..16: mol_type = other RNA
gttttgttaccatatg

SEQ ID NO: 28 - RNA, 18 nt, synthetic construct
  misc_feature 1..18: note = Synthetic
  source 1..18: mol_type = other RNA
tatatgacctaacaaaac

SEQ ID NO: 29 - RNA, 19 nt, synthetic construct
  misc_feature 1..19: note = Synthetic
  source 1..19: mol_type = other RNA
gttttgttaccatatgatt

SEQ ID NO: 30 - RNA, 21 nt, synthetic construct
  misc_feature 1..21: note = Synthetic
  source 1..21: mol_type = other RNA
atttatatgacctaacaaaac

SEQ ID NO: 31 - RNA, 38 nt, synthetic construct
  misc_feature 1..38: note = Synthetic
  source 1..38: mol_type = other RNA
aagggtttatcccggactcggctcttcggagccttttt

SEQ ID NO: 32 - RNA, 11 nt, synthetic construct
  misc_feature 1..11: note = Synthetic
  source 1..11: mol_type = other RNA
gggtttatccc

SEQ ID NO: 33 - RNA, 14 nt, synthetic construct
  misc_feature 1..14: note = Synthetic
  source 1..14: mol_type = other RNA
ggctcttcggagcc

SEQ ID NO: 34 - RNA, 76 nt, synthetic construct
  misc_feature 1..76: note = Synthetic
  source 1..76: mol_type = other RNA
gttttgttaccatatggaaatatatgacctaacaaaacaagggtttatcccggactcggctcttcggagccttttt

SEQ ID NO: 35 - RNA, 82 nt, synthetic construct
  misc_feature 1..82: note = Synthetic
  source 1..82: mol_type = other RNA
gttttgttaccatatgattgaaaatttatatgacctaacaaaacaagggtttatcccggactcggctcttcggagccttttt

SEQ ID NO: 36 - RNA, 82 nt, synthetic construct
  misc_feature 1..82: note = Synthetic
  source 1..82: mol_type = other RNA
gtttagttaccatatgattgaaaatttatatgacctaactaaacaagggtttatcccggactcggctcttcggagccttttt

SEQ ID NO: 37 - RNA, 14 nt, synthetic construct
  misc_feature 1..14: note = Synthetic
  source 1..14: mol_type = other RNA
gtttgagagttatg

SEQ ID NO: 38 - RNA, 16 nt, synthetic construct
  misc_feature 1..16: note = Synthetic
  source 1..16: mol_type = other RNA
catgacgagttcaaat

SEQ ID NO: 39 - RNA, 17 nt, synthetic construct
  misc_feature 1..17: note = Synthetic
  source 1..17: mol_type = other RNA
gtttgagagttatgtaa

SEQ ID NO: 40 - RNA, 19 nt, synthetic construct
  misc_feature 1..19: note = Synthetic
  source 1..19: mol_type = other RNA
ttacatgacgagttcaaat

SEQ ID NO: 41 - RNA, 72 nt, synthetic construct
  misc_feature 1..72: note = Synthetic
  source 1..72: mol_type = other RNA
aaaaatttattcaaaccgcctatttataggccgcagatgttctgcattatgcttgctattgcaagctttttt

SEQ ID NO: 42 - RNA, 14 nt, synthetic construct
  misc_feature 1..14: note = Synthetic
  source 1..14: mol_type = other RNA
gcctatttataggc

SEQ ID NO: 43 - RNA, 34 nt, synthetic construct
  misc_feature 1..34: note = Synthetic
  source 1..34: mol_type = other RNA
gcagatgttctgcattatgcttgctattgcaagc

SEQ ID NO: 44 - RNA, 106 nt, synthetic construct
  misc_feature 1..106: note = Synthetic
  source 1..106: mol_type = other RNA
gtttgagagttatggaaacatgacgagttcaaataaaaatttattcaaaccgcctatttataggccgcagatgttctgcattatgcttgctattgcaagctttttt

SEQ ID NO: 45 - RNA, 112 nt, synthetic construct
  misc_feature 1..112: note = Synthetic
  source 1..112: mol_type = other RNA
gtttgagagttatgtaagaaattacatgacgagttcaaataaaaatttattcaaaccgcctatttataggccgcagatgttctgcattatgcttgctattgcaagctttttt

SEQ ID NO: 46 - RNA, 14 nt, synthetic construct
  misc_feature 1..14: note = Synthetic
  source 1..14: mol_type = other RNA
gtttgagaaccatg

SEQ ID NO: 47 - RNA, 16 nt, synthetic construct
  misc_feature 1..16: note = Synthetic
  source 1..16: mol_type = other RNA
catggtgagtgcaaat

SEQ ID NO: 48 - RNA, 17 nt, synthetic construct
  misc_feature 1..17: note = Synthetic
  source 1..17: mol_type = other RNA
gtttgagaaccatgtaa

SEQ ID NO: 49 - RNA, 19 nt, synthetic construct
  misc_feature 1..19: note = Synthetic
  source 1..19: mol_type = other RNA
ttacatggtgagtgcaaat

SEQ ID NO: 50 - RNA, 64 nt, synthetic construct
  misc_feature 1..64: note = Synthetic
  source 1..64: mol_type = other RNA
aaggattatccgaaattgtatgcccgcattgtgcggcaataaaaaggctcgaaagagtcttttt

SEQ ID NO: 51 - RNA, 64 nt, synthetic construct; identical to SEQ ID NO: 50
  misc_feature 1..64: note = Synthetic
  source 1..64: mol_type = other RNA

SEQ ID NO: 52 - RNA, 46 nt, synthetic construct
  misc_feature 1..46: note = Synthetic
  source 1..46: mol_type = other RNA
tgtatgcccgcattgtgcggcaataaaaaggctcgaaagagtcttt

SEQ ID NO: 53 - RNA, 98 nt, synthetic construct
  misc_feature 1..98: note = Synthetic
  source 1..98: mol_type = other RNA
gtttgagaaccatggaaacatggtgagtgcaaataaggattatccgaaattgtatgcccgcattgtgcggcaataaaaaggctcgaaagagtcttttt

SEQ ID NO: 54 - RNA, 104 nt, synthetic construct
  misc_feature 1..104: note = Synthetic
  source 1..104: mol_type = other RNA
gtttgagaaccatgtaagaaattacatggtgagtgcaaataaggattatccgaaattgtatgcccgcattgtgcggcaataaaaaggctcgaaagagtcttttt

SEQ ID NO: 55 - DNA, 22 bp, synthetic construct
  misc_feature 1..22: note = Synthetic
  source 1..22: mol_type = other DNA
ggtgcggttcaccagggtgtcg

SEQ ID NO: 56 - DNA, 22 bp, synthetic construct
  misc_feature 1..22: note = Synthetic
  source 1..22: mol_type = other DNA
ggaagagcagagccttggtctc

SEQ ID NO: 57 - DNA, 6670 bp, synthetic construct
  misc_feature 1..6670: note = Synthetic
  source 1..6670: mol_type = other DNA
gttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtcgggatctcgacgctaaattaatacgactcactataggggaattgtgagcggataacaattcccctgtagaaataattttgtttaactaaagaggagaaatttcatatgtacccatacgatgtgccagattacgctggcaccgagctcggtaccggctatacaattggcctggatctgggcgttgcctctcttggctgggccgtcgtgaatgatgagtacgaggtgctggaaagctgcagcaacatctttcctgccgccgagagcgccaacaacgtggaaagaagaggcttccggcaaggcagacggctgagcagaagaagaaggacccggatcagcgacttcagaaagctgtgggagaagtccggcttcgaggtgcccagcaatgagctgaatgaggtgctgcagtaccggatcaagggcatgaacgacaagctgagcgaggacgagctgtaccacgtgctgctgaacagcctgaagcacagaggcatcagctacctggacgacgccgatgatgagaacgcctctggcgattatgccgcctctatcgcctacaacgagaaccagctgaaaacaaagctgccctgcgagatccagtgggagagatacaagaagtacggcgcctaccggggcaacatcacaatccaagaaggcggcgagcccctgacactgagaaatgtgtttaccaccagcgcctacgagaaagagatccagaaactgctggacgtgcagagcatgagcaacgagaaagtgaccaagaagttcatcgacgagtacctcaagatcttcagccggaagagagagtactacatcggccctggcaacaagaagtccagaaccgactacggcgtgtacaccacacagaagaacgaggacggcacctaccacaccgagcagaacctgttcgataagctgatcggcaagtgcagcgtgtaccctgatgagcgtagagccgctggcgccacatacacagcccaagagttcaacctgctgaacgatctgaacaacctggtcatcgacggccggaagctggacgagcaagagaagtgtcagatcgtggatgccgtgaagcacgccaagaccgtgaacatgaagaacatcattgccaaagtgatcggcaccaaggccaacagcatgaacatgaccggcgccagaatcgacaagaatgagaaagaaatcttccacagcttcgaggcctacaacaagctgcggaaggccctggaagagatcgacttcgacatcgagacactgagcaccgacgagctggatgccattggagaggtgctgaccctgaacaccgaccggaagtctatccagaacggcctgcaagagaaacggatcgtggtgcccgatgaagtgcgggatgtgctgatcgccaccagaaagagaaatggcagcctgttctccaagtggcagagcttcggcatccggatcatgaaggaactgatcccagagctgtacgcccagcctaagaaccagatgcagctgctgaccgacatgggcgtgttcaagaccaaggacgagagattcgtggaatacgacaagatccccagcgacctgatcaccgaagagatctacaaccccgtggtggccaagacagtgcggatcaccgttagagtgctgaacgccctgatcaagaagtatggctaccccgaccgggtcgtgatcgagatgcccagagataagaactccgaggaagagaagaagcggatcgccgacttccagaagaacaacgaaaacgagcttggcggcatcatcaagaaagtgaagtccgagtacggcatcgagatcaccgacgccgactttaagaaccacagcaagctgggcctgaagctgagactgtggaacgagcagaatgagacatgcccctacagcggcaagcacatcaagatcgacgacctgctcaacaaccccaacatgttcgaggtggaccacatcatccctctgagcatcagcttcgacgacagcagagccaacaaggtgctggtgtacgccgccgaaaaccagaacaagggcaacagaacccctatggcctacctgagcaacgtgaacagagagtgggacttccacgagtacatgagcttcgtgctgagcaactacaagggcaccatctacggcaagaagcgggacaatctgctgttctccgaggacatctacaagatcgatgtgctgcagggcttcatctcccggaacatcaacgacaccagatacgcctctaaagtgatcctgaactccctgcagagctttttcggcagcaaagaatgcgacaccaaagtgaaggtcgtgcggggcaccttcacacaccagatgcggatgaacctgaagatcgagaagaaccgggaagagtcctacgtgcaccacgccgtggatgctatgctgattgccttcagccagatgggctacgacgcctaccacaaactgaccgagaagtatatcgactacgagcacggcgagttcgtggaccagaagggatacgagaagctgattgagaacgacgtggcctacagagagacaacctatcagaacaagtggatgaccatcaagaagaatatcgagatcgccgctgagaaaaacaagtactggtatcaagtgaatcggaagtccaaccggggcctgtgcaaccagaccatctatggcaccagaaacctggacggcaaaaccgtgaagatctccaagctggacatccggaccgacgacggcatcaaaaagtttaagggcatcgtggaaaagggcaagctggaacggttcctgatgtaccggaacgaccccaagaccttcgagtggctgctgcagatctataaggactacagcgacagcaagaaccccttcgtgcagtacgagtctgagacaggcgacgtgatcaaaaaggtgtccaagacaaacaacggccccaaagtgtgcgagctgagatacgaggatggcgaagtgggctcctgcatcgacatcagccacaaatacggctacaagaagggcagcaagaaagtcatcctggattctctgaacccctaccggatggacgtgtactacaacaccaaggacaaccggtactacttcgtgggcgtgaagtactccgacatcaagtgccagggcgacagctacgtgatcgacgaggataagtatgccgccgctctggtgcaagaaaagatcgtgccagaaggcaagggcagatccgatctgaccgagctgggctatgagttcaagctgtccttctacaagaacgagatcatcgagtacgagaaggacggggagatctacgtcgagcggttcctgtccagaacaatgcctaaagtgtccaactatatcgagacaaagcccctggaagccgccaagttcgagaagagaaacctcgtgggcctcgccaagacaagccggatcagaaagatcagagtggacatcctggggaaccgctacctgaacagcatggaaaacttcgacttcgtcgtgggccacaagggatcctaagcggccgcctagcataaccccttggggcctctaaacgggtcttgaggggttttttgacctaggctaggggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgttactagtgcttggattctcaccaataaaaaacgcccggcggcaaccgagcgttctgaacaaatccagatggagttctgaggtcattactggatctatcaacaggagtccaagcgagctcgtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgggagccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcctcgagtcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgacacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagtt

SEQ ID NO: 58 - DNA, 2567 bp, synthetic construct
  misc_feature 1..2567: note = Synthetic
  source 1..2567: mol_type = other DNA
gcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaacacatttccccgaaaagtgccacctgacgtcctcgagtcccgcataatcgaaatttgacagctagctcagtcctaggtataatactagtggaagagcagagccttggtctcgttttagtacctagagaaagaaatttctttagacctactaaaataaggctttatgccgagattaaaggatgccgacgggcatccttttttgaattctcaaataaaacgaaaggctcagtcgaaagactgggcctttcgttttatctgttgtttgtcggtgaacgctctcctgagtaggacaaatggtaccccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgttactagtgcttggattctcaccaataaaaaacgcccggcggcaaccgagcgttctgaacaaatccagatggagttctgaggtcattactggatctatcaacaggagtccaagcgagaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccg

SEQ ID NO: 59 - DNA, 5009 bp, synthetic construct
  misc_feature 1..5009: note = Synthetic
  misc_feature 3040..3047: note = n is a, c, g, or t
  source 1..5009: mol_type = other DNA
tcgagtctttacactttatgcttccggctcgtatgttgtgtggaattgtgagcggataacaatttcacacatgattacggattcaacgtcgtgactggtaaaacccgggcgttacccaacttaatcgccttgcagcacatccccctttcgccagcaggcgtaataaggaaaggattcatgtactatttgaaaaacacaaacttttggatgttcggtttattctttttcttttacttttttatcatgggagcctacttcccgtttttcccgatttggctacatgatatcaaccatatcagcaaaagtgatacgggtattatttttgccgctatttctctgttctcgctattattccaaccgctgtttggtctgctttctgacaaactcggtctacgcaaatacctgctgtggattattaccggcatgttagtgatgtttgcgccgttctttatttttatcttcgggccactgctgcagtacaacattttagtagggtcgattgttggtggtatttatctaggctttagttttaacgccggtgcgccagcagtagaggcatttattgagaaagtcagccggcgcagtaatttcgaatttggtcgcgcgcggatgtttggcagtgttggctgggcgctggttgcctcgattgtcgggatcatgttcaccattaataatcagtttgttttctggctgggctctggcagttgtctcatcctcgccgttttactctttttcgccaaaacggacgcgccctcaagtgccacggttgccaatgcggtaggtgccaaccattcggcatttagccttaagctggcactggaactgttcagacagccaaaactgtggtttttgtcactgtatgttattggcgtttcctccacctacgatgtttttgaccaacagtttgctaatttctttacttcgttctttgctaccggtgaacagggtacccgcgtatttggctacgtaacgacaatgggcgaattacttaacgcctcgattatgttctttgcgccactgatcattaatcgcatcggtgggaagaatgccctgctgctggctggcactattatgtctgtacgtattattggctcatcgttcgccacctcagcgctggaagtggttattctgaaaacgctgcatatgtttgaagtaccgttcctgctggtgggctcctttaaatatattactagtcagtttgaagtgcgtttttcagcgacgatttatctggtcagtttcagcttctttaagcaactggcgatgatttttatgtctgtactggcgggcaatatgtatgaaagcataggtttccaaggcgcttatctggtgctgggtctggtggcgctgggcttcaccttaatttccgtgttcacgcttagcggcccgggcccgctttccctgctgcgtcgtcaggtgaatgaagtcgcttaaaggcctcgatgcagctagcatgctaatctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttggggtagcgattgaaaacgatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttttggggaatataatcttctagacatacaatggaagagcagagccttggtctcnnnnnnnnaagcttgatatcgaattcctgcagcccgggggatcccatggtacgcgtgctagaggcatcaaataaaacgaaaggctcagtcgaaagactgggcctttcgttttatctgttgtttgtcggtgaacgctctcctgagtaggacaaatccgccgccctagacctaggcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgactagtgcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccagatatcgacgtctaagaaaccattattatcatgacattaacctataaaaataggcgtatcacgaggccctttcgtcttcacc

SEQ ID NO: 60 - DNA, 6439 bp, synthetic construct
  misc_feature 1..6439: note = Synthetic
  source 1..6439: mol_type = other DNA
taatacgactcactatagggagaccacaacggtttccctctagagagacaataaccctgataatgcttcaataatattgaaaaaggaagagtatgcctaagaagaagagaaaggtgggtaccaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgagaacctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgcttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctgggatcctacccatacgatgttccagattacgcggccgctccaaaaaagaaaagaaaagttgcggctagccatcatcaccatcaccatcatcattaaggctgctaacaaagcccgaaaggaagctgagttggctgctgccaccgctgagcaataactagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttcgatcccgcgaaat

SEQ ID NO: 61 - DNA, 9542 bp, synthetic construct
  misc_feature 1..9542: note = Synthetic
  source 1..9542: mol_type = other DNA
ggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttgccaccatgcctaagaagaagagaaaggtgggtaccggctatacaattggcctggatctgggcgttgcctctcttggctgggccgtcgtgaatgatgagtacgaggtgctggaaagctgcagcaacatctttcctgccgccgagagcgccaacaacgtggaaagaagaggcttccggcaaggcagacggctgagcagaagaagaaggacccggatcagcgacttcagaaagctgtgggagaagtccggcttcgaggtgcccagcaatgagctgaatgaggtgctgcagtaccggatcaagggcatgaacgacaagctgagcgaggacgagctgtaccacgtgctgctgaacagcctgaagcacagaggcatcagctacctggacgacgccgatgatgagaacgcctctggcgattatgccgcctctatcgcctacaacgagaaccagctgaaaacaaagctgccctgcgagatccagtgggagagatacaagaagtacggcgcctaccggggcaacatcacaatccaagaaggcggcgagcccctgacactgagaaatgtgtttaccaccagcgcctacgagaaagagatccagaaactgctggacgtgcagagcatgagcaacgagaaagtgaccaagaagttcatcgacgagtacctcaagatcttcagccggaagagagagtactacatcggccctggcaacaagaagtccagaaccgactacggcgtgtacaccacacagaagaacgaggacggcacctaccacaccgagcagaacctgttcgataagctgatcggcaagtgcagcgtgtaccctgatgagcgtagagccgctggcgccacatacacagcccaagagttcaacctgctgaacgatctgaacaacctggtcatcgacggccggaagctggacgagcaagagaagtgtcagatcgtggatgccgtgaagcacgccaagaccgtgaacatgaagaacatcattgccaaagtgatcggcaccaaggccaacagcatgaacatgaccggcgccagaatcgacaagaatgagaaagaaatcttccacagcttcgaggcctacaacaagctgcggaaggccctggaagagatcgacttcgacatcgagacactgagcaccgacgagctggatgccattggagaggtgctgaccctgaacaccgaccggaagtctatccagaacggcctgcaagagaaacggatcgtggtgcccgatgaagtgcgggatgtgctgatcgccaccagaaagagaaatggcagcctgttctccaagtggcagagcttcggcatccggatcatgaaggaactgatcccagagctgtacgcccagcctaagaaccagatgcagctgctgaccgacatgggcgtgttcaagaccaaggacgagagattcgtggaatacgacaagatccccagcgacctgatcaccgaagagatctacaaccccgtggtggccaagacagtgcggatcaccgttagagtgctgaacgccctgatcaagaagtatggctaccccgaccgggtcgtgatcgagatgcccagagataagaactccgaggaagagaagaagcggatcgccgacttccagaagaacaacgaaaacgagcttggcggcatcatcaagaaagtgaagtccgagtacggcatcgagatcaccgacgccgactttaagaaccacagcaagctgggcctgaagctgagactgtggaacgagcagaatgagacatgcccctacagcggcaagcacatcaagatcgacgacctgctcaacaaccccaacatgttcgaggtggaccacatcatccctctgagcatcagcttcgacgacagcagagccaacaaggtgctggtgtacgccgccgaaaaccagaacaagggcaacagaacccctatggcctacctgagcaacgtgaacagagagtgggacttccacgagtacatgagcttcgtgctgagcaactacaagggcaccatctacggcaagaagcgggacaatctgctgttctccgaggacatctacaagatcgatgtgctgcagggcttcatctcccggaacatcaacgacaccagatacgcctctaaagtgatcctgaactccctgcagagctttttcggcagcaaagaatgcgacaccaaagtgaaggtcgtgcggggcaccttcacacaccagatgcggatgaacctgaagatcgagaagaaccgggaagagtcctacgtgcaccacgccgtggatgctatgctgattgccttcagccagatgggctacgacgcctaccacaaactgaccgagaagtatatcgactacgagcacggcgagttcgtggaccagaagggatacgagaagctgattgagaacgacgtggcctacagagagacaacctatcagaacaagtggatgaccatcaagaagaatatcgagatcgccgctgagaaaaacaagtactggtatcaagtgaatcggaagtccaaccggggcctgtgcaaccagaccatctatggcaccagaaacctggacggcaaaaccgtgaagatctccaagctggacatccggaccgacgacggcatcaaaaagtttaagggcatcgtggaaaagggcaagctggaacggttcctgatgtaccggaacgaccccaagaccttcgagtggctgctgcagatctataaggactacagcgacagcaagaaccccttcgtgcagtacgagtctgagacaggcgacgtgatcaaaaaggtgtccaagacaaacaacggccccaaagtgtgcgagctgagatacgaggatggcgaagtgggctcctgcatcgacatcagccacaaatacggctacaagaagggcagcaagaaagtcatcctggattctctgaacccctaccggatggacgtgtactacaacaccaaggacaaccggtactacttcgtgggcgtgaagtactccgacatcaagtgccagggcgacagctacgtgatcgacgaggataagtatgccgccgctctggtgcaagaaaagatcgtgccagaaggcaagggcagatccgatctgaccgagctgggctatgagttcaagctgtccttctacaagaacgagatcatcgagtacgagaaggacggggagatctacgtcgagcggttcctgtccagaacaatgcctaaagtgtccaactatatcgagacaaagcccctggaagccgccaagttcgagaagagaaacctcgtgggcctcgccaagacaagccggatcagaaagatcagagtggacatcctggggaaccgctacctgaacagcatggaaaacttcgacttcgtcgtgggccacaagggatcctacccatacgatgttccagattacgcggccgctccaaaaaagaaaagaaaagttgaattcggcggcagcggcgccaccaacttcagcctgctgaagcaggccggcgacgtggaggagaaccccggccccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttgga

SEQ ID NO: 62 - DNA, 2726 bp, synthetic construct
  misc_feature 1..2726: note = Synthetic
  source 1..2726: mol_type = other DNA
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgccaagtgataaacacgaggagtttaagtacctagagaaagaaatttctttagacctacttaaataaggctttatgccgagattaaaggatgccgacgggcatccttttttgaattctcaaataaaacgaaaggctcagtcgaaagactgggcctttcgttttatctgttgtttgtcggtgaacgctctcctgagtaggacaaatggtaccccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgttactagtgcttggattctcaccaataaaaaacgcccggcggcaaccgagcgttctgaacaaatccagatggagttctgaggtcattactggatctatcaacaggagtccaagcgagaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaa

SEQ ID NO: 63 - protein, 9 aa, synthetic construct
  REGION 1..9: note = Synthetic
YPYDVPDYA

SEQ ID NO: 64 - DNA, 27 bp, synthetic construct
  misc_feature 1..27: note = Synthetic
  source 1..27: mol_type = other DNA
tacccatacgatgttccagattacgct

SEQ ID NO: 65 - protein, 7 aa, synthetic construct
  REGION 1..7: note = Synthetic
PKKKRKV

SEQ ID NO: 66 - DNA, 21 bp, synthetic construct
  misc_feature 1..21: note = Synthetic
  source 1..21: mol_type = other DNA
ccaaaaaagaaaagaaaagtt

SEQ ID NO: 67 - protein, 236 aa, synthetic construct
  REGION 1..236: note = Synthetic
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 68 - DNA, 711 bp, synthetic construct
  misc_feature 1..711: note = Synthetic
  source 1..711: mol_type = other DNA
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtag

SEQ ID NO: 69 - protein, 7 aa, Simian virus 40
PKKKRKV

SEQ ID NO: 70 - protein, 16 aa, unidentified
  REGION 1..16: note = Synthetic
KRPAATKKAGQAKKKK

SEQ ID NO: 71 - protein, 7 aa, unidentified
  REGION 1..7: note = Synthetic
PAARVLD

SEQ ID NO: 72 - protein, 11 aa, unidentified
  REGION 1..11: note = Synthetic
RQRRNELKRSP

SEQ ID NO: 73 - protein, 37 aa, Homo sapiens
NQSSNFGPMGGNFGGRSSGPYGGGGQYFAKPRNQGGY

SEQ ID NO: 74 - protein, 42 aa, unidentified
  REGION 1..42: note = Synthetic
RMRIZFKNKGKDTAELRRRRVEVSVELRKAKKDEQILKRRNV

SEQ ID NO: 75 - protein, 8 aa, unidentified
  REGION 1..8: note = Synthetic
VSRKRPRP

SEQ ID NO: 76 - protein, 8 aa, unidentified
  REGION 1..8: note = Synthetic
PPKKARED

SEQ ID NO: 77 - protein, 7 aa, Homo sapiens
PQPKKPL

SEQ ID NO: 78 - protein, 9 aa, Mus musculus
SAIIKKKKM

SEQ ID NO: 79 - protein, 5 aa, Influenza virus
DRLRR

SEQ ID NO: 80 - protein, 7 aa, Influenza virus
PKQKKRK

SEQ ID NO: 81 - protein, 10 aa, Hepatitis delta virus
RKLKKKIKKL

SEQ ID NO: 82 - protein, 10 aa, Mus musculus
REKKKFLKRR

SEQ ID NO: 83 - protein, 19 aa, Homo sapiens
KRGDEVDGVDEVAKKKSKK

SEQ ID NO: 84 - protein, 17 aa, Homo sapiens
RKCLQAGMNLEARKTKK

SEQ ID NO: 85 - protein, 19 aa, synthetic construct
  REGION 1..19: note = Synthetic
ATNFSLLKQAGDVEENPGP

SEQ ID NO: 86 - DNA, 57 bp, synthetic construct
  misc_feature 1..57: note = Synthetic
  source 1..57: mol_type = other DNA
gccaccaacttcagcctgctgaagcaggccggcgacgtggaggagaaccccggcccc

SEQ ID NO: 87 - RNA, 108 nt, synthetic construct
  misc_feature 1..108: note = Synthetic
  source 1..108: mol_type = other RNA
gtttgagagttatgtgaaaacatgacgagttcaaataaaaatttattcaaaccgcctatttataggccgcagatgttctgcattatgcttgctattgcaagctttttt

SEQ ID NO: 88 - RNA, 110 nt, synthetic construct
  misc_feature 1..110: note = Synthetic
  source 1..110: mol_type = other RNA
gtttgagagttatgtagaaatacatgacgagttcaaataaaaatttattcaaaccgcctatttataggccgcagatgttctgcattatgcttgctattgcaagctttttt

SEQ ID NO: 89 - RNA, 20 nt, synthetic construct
  misc_feature 1..20: note = Synthetic
  source 1..20: mol_type = other RNA
ccaagtgataaacacgagga

SEQ ID NO: 90 - RNA, 21 nt, synthetic construct
  misc_feature 1..21: note = Synthetic
  source 1..21: mol_type = other RNA
gtcacctccaatgactagggt

SEQ ID NO: 91 - RNA, 20 nt, synthetic construct
  misc_feature 1..20: note = Synthetic
  source 1..20: mol_type = other RNA
gccgccattgacagagggac

SEQ ID NO: 92 - RNA, 20 nt, synthetic construct
  misc_feature 1..20: note = Synthetic
  source 1..20: mol_type = other RNA
aactggtaccgcatgagccc

SEQ ID NO: 93 - RNA, 20 nt, synthetic construct
  misc_feature 1..20: note = Synthetic
  source 1..20: mol_type = other RNA
catcaggctctcagctcagc

SEQ ID NO: 94 - RNA, 20 nt, synthetic construct
  misc_feature 1..20: note = Synthetic
  source 1..20: mol_type = other RNA
aggtgccgtttgttcatttt

SEQ ID NO: 95 - RNA, 20 nt, synthetic construct
  misc_feature 1..20: note = Synthetic
  source 1..20: mol_type = other RNA
ccagttgtagcaccgcccag

SEQ ID NO: 96 - RNA, 20 nt, synthetic construct
  misc_feature 1..20: note = Synthetic
  source 1..20: mol_type = other RNA
tctccccagccctgctcgtg

SEQ ID NO: 97 - RNA, 20 nt, synthetic construct
  misc_feature 1..20: note = Synthetic
  source 1..20: mol_type = other RNA
tctgtgaatgttagacccat

SEQ ID NO: 98 - RNA, 20 nt, synthetic construct
  misc_feature 1..20: note = Synthetic
  source 1..20: mol_type = other RNA
ccatgggagcagctggtcag

SEQ ID NO: 99 - RNA, 20 nt, synthetic construct
  misc_feature 1..20: note = Synthetic
  source 1..20: mol_type = other RNA
gcaagagacccacacaccgg

SEQ ID NO: 100 - RNA, 20 nt, synthetic construct
  misc_feature 1..20: note = Synthetic
  source 1..20: mol_type = other RNA
acaccggaggagcgcccgct

SEQ ID NO: 101 - RNA, 20 nt, synthetic construct
  misc_feature 1..20: note = Synthetic
  source 1..20: mol_type = other RNA
cgtctgggcggtgctacaac

SEQ ID NO: 102 - RNA, 20 nt, synthetic construct
  misc_feature 1..20: note = Synthetic
  source 1..20: mol_type = other RNA
ctacaactgggctggcggcc

SEQ ID NO: 103 - RNA, 20 nt, synthetic construct
  misc_feature 1..20: note = Synthetic
  source 1..20: mol_type = other RNA
agtccgggctgggagcgggt

SEQ ID NO: 104 - RNA, 20 nt, synthetic construct
  misc_feature 1..20: note = Synthetic
  source 1..20: mol_type = other RNA
gctgcgggaaagggattccc

SEQ ID NO: 105 - RNA, 20 nt, synthetic construct
  misc_feature 1..20: note = Synthetic
  source 1..20: mol_type = other RNA
acagcgggtgtagactccga

SEQ ID NO: 106 - RNA, 20 nt, synthetic construct
  misc_feature 1..20: note = Synthetic
  source 1..20: mol_type = other RNA
cagcgggtgtagactccgag

SEQ ID NO: 107 - RNA, 20 nt, synthetic construct
  misc_feature 1..20: note = Synthetic
  source 1..20: mol_type = other RNA
gtcaagccccagaggccaca

SEQ ID NO: 108 - RNA, 20 nt, synthetic construct
  misc_feature 1..20: note = Synthetic
  source 1..20: mol_type = other RNA
gcctggggcccctaacccta

SEQ ID NO: 109 - RNA, 20 nt, synthetic construct
  misc_feature 1..20: note = Synthetic
  source 1..20: mol_type = other RNA
attttctgacactcccgccc

SEQ ID NO: 110 - RNA, 20 nt, synthetic construct
  misc_feature 1..20: note = Synthetic
  source 1..20: mol_type = other RNA
atcctggccgccagcccagt

SEQ ID NO: 111 - RNA, 20 nt, synthetic construct
  misc_feature 1..20: note = Synthetic
  source 1..20: mol_type = other RNA
ggagagcttcgtgctaaact

SEQ ID NO: 112 - RNA, 23 nt, synthetic construct
  misc_feature 1..23: note = Synthetic
  source 1..23: mol_type = other RNA
tgcagtccgggctgggagcgggt

SEQ ID NO: 113 - RNA, 135 nt, synthetic construct
  misc_feature 1..135: note = Synthetic
  source 1..135: mol_type = other RNA
tgcagtccgggctgggagcgggtgtttgagagttatgtaagaaattacatgacgagttcaaataaaaatttattcaaaccgcctatttataggccgcagatgttctgcattatgcttgctattgcaagctttttt

SEQ ID NO: 114 - DNA, 43 bp, synthetic construct
  misc_feature 1..43: note = Synthetic
  source 1..43: mol_type = other DNA
ctgttgctgcagtccgggctgggagcgggtggggagcagaggg

SEQ ID NO: 115 - DNA, 43 bp, synthetic construct
  misc_feature 1..43: note = Synthetic
  source 1..43: mol_type = other DNA
gttaagagacagtccaggctgggagcaggtggggagaggaggg

SEQ ID NO: 116 - RNA, 22 nt, synthetic construct
  misc_feature 1..22: note = Synthetic
  source 1..22: mol_type = other RNA
gcagtccgggctgggagcgggt

SEQ ID NO: 117 - RNA, 134 nt, synthetic construct
  misc_feature 1..134: note = Synthetic
  source 1..134: mol_type = other RNA
gcagtccgggctgggagcgggtgtttgagagttatgtaagaaattacatgacgagttcaaataaaaatttattcaaaccgcctatttataggccgcagatgttctgcattatgcttgctattgcaagctttttt

SEQ ID NO: 118 - RNA, 21 nt, synthetic construct
  misc_feature 1..21: note = Synthetic
  source 1..21: mol_type = other RNA
cagtccgggctgggagcgggt

SEQ ID NO: 119 - RNA, 133 nt, synthetic construct
  misc_feature 1..133: note = Synthetic
  source 1..133: mol_type = other RNA
cagtccgggctgggagcgggtgtttgagagttatgtaagaaattacatgacgagttcaaataaaaatttattcaaaccgcctatttataggccgcagatgttctgcattatgcttgctattgcaagctttttt

SEQ ID NO: 120 - RNA, 20 nt, synthetic construct
  misc_feature 1..20: note = Synthetic
  source 1..20: mol_type = other RNA
agtccgggctgggagcgggt

SEQ ID NO: 121 - RNA, 132 nt, synthetic construct
  misc_feature 1..132: note = Synthetic
  source 1..132: mol_type = other RNA
agtccgggctgggagcgggtgtttgagagttatgtaagaaattacatgacgagttcaaataaaaatttattcaaaccgcctatttataggccgcagatgttctgcattatgcttgctattgcaagctttttt

SEQ ID NO: 122 - RNA, 19 nt, synthetic construct
  misc_feature 1..19: note = Synthetic
  source 1..19: mol_type = other RNA
gtccgggctgggagcgggt

SEQ ID NO: 123 - RNA, 131 nt, synthetic construct
  misc_feature 1..131: note = Synthetic
  source 1..131: mol_type = other RNA
gtccgggctgggagcgggtgtttgagagttatgtaagaaattacatgacgagttcaaataaaaatttattcaaaccgcctatttataggccgcagatgttctgcattatgcttgctattgcaagctttttt

SEQ ID NO: 124 - RNA, 18 nt, synthetic construct
  misc_feature 1..18: note = Synthetic
  source 1..18: mol_type = other RNA
tccgggctgggagcgggt

SEQ ID NO: 125 - RNA, 130 nt, synthetic construct
  misc_feature 1..130: note = Synthetic
  source 1..130: mol_type = other RNA
tccgggctgggagcgggtgtttgagagttatgtaagaaattacatgacgagttcaaataaaaatttattcaaaccgcctatttataggccgcagatgttctgcattatgcttgctattgcaagctttttt

SEQ ID NO: 126 - RNA, 17 nt, synthetic construct
  misc_feature 1..17: note = Synthetic
  source 1..17: mol_type = other RNA
ccgggctgggagcgggt

SEQ ID NO: 127 - RNA, 129 nt, synthetic construct
  misc_feature 1..129: note = Synthetic
  source 1..129: mol_type = other RNA
ccgggctgggagcgggtgtttgagagttatgtaagaaattacatgacgagttcaaataaaaatttattcaaaccgcctatttataggccgcagatgttctgcattatgcttgctattgcaagctttttt

SEQ ID NO: 128 - RNA, 128 nt, synthetic construct
  misc_feature 1..128: note = Synthetic
  source 1..128: mol_type = other RNA
agtccgggctgggagcgggtgtttgagagttatgtgaaaacatgacgagttcaaataaaaatttattcaaaccgcctatttataggccgcagatgttctgcattatgcttgctattgcaagctttttt

SEQ ID NO: 129 - RNA, 130 nt, synthetic construct
  misc_feature 1..130: note = Synthetic
  source 1..130: mol_type = other RNA
agtccgggctgggagcgggtgtttgagagttatgtagaaatacatgacgagttcaaataaaaatttattcaaaccgcctatttataggccgcagatgttctgcattatgcttgctattgcaagctttttt

SEQ ID NO: 130 - DNA, 28 bp, synthetic construct
  misc_feature 1..28: note = Synthetic
  source 1..28: mol_type = other DNA
agtccgggctgggagcgggtggggagca

SEQ ID NO: 131 - DNA, 28 bp, synthetic construct
  misc_feature 1..28: note = Synthetic
  source 1..28: mol_type = other DNA
gtcaagccccagaggccacagggacaga

SEQ ID NO: 132 - DNA, 28 bp, synthetic construct
  misc_feature 1..28: note = Synthetic
  source 1..28: mol_type = other DNA
agtcctggctgggagcaggtggggagag

SEQ ID NO: 133 - DNA, 28 bp, synthetic construct
  misc_feature 1..28: note = Synthetic
  source 1..28: mol_type = other DNA
gccaagcctcagaggccacagggcagca

SEQ ID NO: 134 - DNA, 28 bp, synthetic construct
  misc_feature 1..28: note = Synthetic
  source 1..28: mol_type = other DNA
ccaagtgataaacacgaggatggcaaga

SEQ ID NO: 135 - DNA, 28 bp, synthetic construct
  misc_feature 1..28: note = Synthetic
  source 1..28: mol_type = other DNA
aactggtaccgcatgagccccagcaacc

SEQ ID NO: 136 - DNA, 28 bp, synthetic construct
  misc_feature 1..28: note = Synthetic
  source 1..28: mol_type = other DNA
catcaggctctcagctcagcctgagtgt

SEQ ID NO: 137 - DNA, 28 bp, synthetic construct
  misc_feature 1..28: note = Synthetic
  source 1..28: mol_type = other DNA
aggtgccgtttgttcattttctgacact

SEQ ID NO: 138 - DNA, 28 bp, synthetic construct
  misc_feature 1..28: note = Synthetic
  source 1..28: mol_type = other DNA
ccagttgtagcaccgcccagacgactgg

SEQ ID NO: 139 - DNA, 28 bp, synthetic construct
  misc_feature 1..28: note = Synthetic
  source 1..28: mol_type = other DNA
tctccccagccctgctcgtggtgaccga

SEQ ID NO: 140 - DNA, 28 bp, synthetic construct
  misc_feature 1..28: note = Synthetic
  source 1..28: mol_type = other DNA
tctgtgaatgttagacccatgggagcag

SEQ ID NO: 141 - DNA, 28 bp, synthetic construct
  misc_feature 1..28: note = Synthetic
  source 1..28: mol_type = other DNA
gcaagagacccacacaccggaggagcgc

SEQ ID NO: 142 - DNA, 28 bp, synthetic construct
  misc_feature 1..28: note = Synthetic
  source 1..28: mol_type = other DNA
acaccggaggagcgcccgcttgggggag

SEQ ID NO: 143 - DNA, 28 bp, synthetic construct
  misc_feature 1..28: note = Synthetic
  source 1..28: mol_type = other DNA
cgtctgggcggtgctacaactgggctgg

SEQ ID NO: 144 - DNA, 28 bp, synthetic construct
  misc_feature 1..28: note = Synthetic
  source 1..28: mol_type = other DNA
gctgcgggaaagggattccctgggactc

SEQ ID NO: 145 - DNA, 28 bp, synthetic construct
  misc_feature 1..28: note = Synthetic
  source 1..28: mol_type = other DNA
gcctggggcccctaaccctatgtagcct

SEQ ID NO: 146 - DNA, 28 bp, synthetic construct
  misc_feature 1..28: note = Synthetic
  source 1..28: mol_type = other DNA
attttctgacactcccgcccaatatacc

SEQ ID NO: 147 - DNA, 28 bp, synthetic construct
  misc_feature 1..28: note = Synthetic
  source 1..28: mol_type = other DNA
atcctggccgccagcccagttgtagcac

SEQ ID NO: 148 - DNA, 28 bp, synthetic construct
  misc_feature 1..28: note = Synthetic
  source 1..28: mol_type = other DNA
ggagagcttcgtgctaaactggtaccgc

SEQ ID NO: 149 - RNA, 11 nt, synthetic construct
  misc_feature 1..11: note = tracrRNA
  source 1..11: mol_type = other RNA
cgagttcaaat

SEQ ID NO: 150 - RNA, 17 nt, synthetic construct
  misc_feature 1..17: note = tracrRNA Portion 1
  source 1..17: mol_type = other RNA
aaaaatttattcaaacc

SEQ ID NO: 151 - RNA, 14 nt, synthetic construct
  misc_feature 1..14: note = tracrRNA Portion 3
  source 1..14: mol_type = other RNA
cgcagatgttctgc

SEQ ID NO: 152 - RNA, 27 nt, synthetic construct
  misc_feature 1..27: note = tracrRNA Portion 4
  source 1..27: mol_type = other RNA
attatgcttgctattgcaagctttttt

SEQ ID NO: 153 - RNA, 88 nt, synthetic construct
  misc_feature 1..88: note = Full tracrRNA V1
  source 1..88: mol_type = other RNA
catgacgagttcaaataaaaatttattcaaaccgcctatttataggccgcagatgttctgcattatgcttgctattgcaagctttttt

SEQ ID NO: 154 - RNA, 91 nt, synthetic construct
  misc_feature 1..91: note = Full tracrRNA V2
  source 1..91: mol_type = other RNA
ttacatgacgagttcaaataaaaatttattcaaaccgcctatttataggccgcagatgttctgcattatgcttgctattgcaagctttttt

SEQ ID NO: 155 - DNA, 4194 bp, synthetic construct
  misc_feature 1..4194: note = pPML3.1
  misc_feature 2256..2261: note = n is a, c, g, or t
  source 1..4194: mol_type = other DNA
gggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgataagcttgggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgactctagaggatccactagtccagtgtggtggaattctgcagatatcaaagcttgccaccatgcatacaatggaagagcagagccttggtctcnnnnnngcgggtctggtggcgctagcgtgtccaagggcgaggagctgttcaccggcgtggtgcccatcctggtggagctggacggcgacgtgaacggccacaagttcagcgtgagcggcgagggcgaaggggacgctacttacggcaaactgactctcaagtttatctgtactaccgggaagctccctgtcccttggcctacactggtcacaactctcacatatggggtccagtgcttcagcagataccccgaccacatgaagcagcacgacttcttcaagagcgccatgcccgagggctacgtgcaggagagaaccatcttcttcaaggacgacggcaactacaagaccagagctgaggtcaagtttgagggtgacaccctggtgaacagaatcgagctgaagggcatcgacttcaaggaggacggcaacatcctgggccacaagctggagtacaactacaacagccacaacgtgtacatcatggctgataaacagaagaatgggattaaggtgaacttcaagatcagacacaacatcgaggacggcagcgtgcagctggccgaccactaccagcagaacacccccatcggcgacggccccgtgctgctgcccgacaaccactacctgagcacccagagcgctctcagtaaggaccctaatgagaagagagaccacatggtgctgctggagttcgtgaccgccgccggcatcaccctgggcatggacgagctgtacaagtgagggcctaatgagtttggaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 156 - DNA, 21 bp, synthetic construct
  misc_feature 1..21: note = TRAC gRNA 1
  source 1..21: mol_type = other DNA
tctctcagctggtacacggca

SEQ ID NO: 157 - DNA, 21 bp, synthetic construct
  misc_feature 1..21: note = TRAC gRNA 2
  source 1..21: mol_type = other DNA
gcgtcatgagcagattaaacc

SEQ ID NO: 158 - DNA, 21 bp, synthetic construct
  misc_feature 1..21: note = TRAC gRNA 3
  source 1..21: mol_type = other DNA
tctcgaccagcttgacatcac

SEQ ID NO: 159 - DNA, 21 bp, synthetic construct
  misc_feature 1..21: note = TRAC gRNA 4
  source 1..21: mol_type = other DNA
ttaaacccggccactttcagg

SEQ ID NO: 160 - DNA, 21 bp, synthetic construct
  misc_feature 1..21: note = TRAC gRNA 5
  source 1..21: mol_type = other DNA
ctgtgctagacatgaggtcta

SEQ ID NO: 161 - DNA, 21 bp, synthetic construct
  misc_feature 1..21: note = TRAC gRNA 8
  source 1..21: mol_type = other DNA
acttcaagagcaacagtgctg

SEQ ID NO: 162 - DNA, 21 bp, synthetic construct
  misc_feature 1..21: note = TRAC gRNA 9
  source 1..21: mol_type = other DNA
aagagcaacagtgctgtggcc

SEQ ID NO: 163 - DNA, 21 bp, synthetic construct
  misc_feature 1..21: note = TRAC gRNA 10
  source 1..21: mol_type = other DNA
gctggggaagaaggtgtcttc

SEQ ID NO: 164 - DNA, 21 bp, synthetic construct
  misc_feature 1..21: note = TRAC gRNA 15
  source 1..21: mol_type = other DNA
ataggcagacagacttgtcac

SEQ ID NO: 165 - DNA, 21 bp, synthetic construct
  misc_feature 1..21: note = TRAC gRNA 17
  source 1..21: mol_type = other DNA
tagagtctctcagctggtaca

SEQ ID NO: 166 - DNA, 21 bp, synthetic construct
  misc_feature 1..21: note = TRACR gRNA 18
  source 1..21: mol_type = other DNA
gtctctcagctggtacacggc

SEQ ID NO: 167 - DNA, 21 bp, synthetic construct
  misc_feature 1..21: note = TRAC gRNA 19
  source 1..21: mol_type = other DNA
cagctggtacacggcagggtc

SEQ ID NO: 168 - DNA, 21 bp, synthetic construct
  misc_feature 1..21: note = TRAC gRNA 20
  source 1..21: mol_type = other DNA
agctggtacacggcagggtca

SEQ ID NO: 169 - DNA, 21 bp, synthetic construct
  misc_feature 1..21: note = TRAC gRNA 21
  source 1..21: mol_type = other DNA
tacacggcagggtcagggttc

SEQ ID NO: 170 - DNA, 21 bp, synthetic construct
  misc_feature 1..21: note = TRAC gRNA 23
  source 1..21: mol_type = other DNA
ctttcaaaacctgtcagtgat

SEQ ID NO: 171 - DNA, 21 bp, synthetic construct
  misc_feature 1..21: note = TRAC gRNA 25
  source 1..21: mol_type = other DNA
tccgaatcctcctcctgaaag

SEQ ID NO: 172 - DNA, 21 bp, synthetic construct
  misc_feature 1..21: note = TRAC gRNA 26
  source 1..21: mol_type = other DNA
aatcctcctcctgaaagtggc

SEQ ID NO: 173 - DNA, 21 bp, synthetic construct
  misc_feature 1..21: note = TRAC gRNA 27
  source 1..21: mol_type = other DNA
atcctcctcctgaaagtggcc

SEQ ID NO: 174 - DNA, 21 bp, synthetic construct
  misc_feature 1..21: note = TRAC gRNA 29
  source 1..21: mol_type = other DNA
ctgctcatgacgctgcggctg

SEQ ID NO: 175 - DNA, 21 bp, synthetic construct
  misc_feature 1..21: note = TRAC gRNA 30
  source 1..21: mol_type = other DNA
agattaaacccggccactttc

SEQ ID NO: 176 - DNA, 21 bp, synthetic construct
  misc_feature 1..21: note = TRAC gRNA 31
  source 1..21: mol_type = other DNA
aacccggccactttcaggagg

SEQ ID NO: 177 - DNA, 21 bp, synthetic construct
  misc_feature 1..21: note = TRAC gRNA 32
  source 1..21: mol_type = other DNA
gccactttcaggaggaggatt

SEQ ID NO: 178 - DNA, 21 bp, synthetic construct
  misc_feature 1..21: note = B2M gRNA 1
  source 1..21: mol_type = other DNA
tactctctctttctggcctgg

SEQ ID NO: 179 - DNA, 21 bp, synthetic construct
  misc_feature 1..21: note = B2M gRNA 2
  source 1..21: mol_type = other DNA
gcatactcatctttttcagtg

SEQ ID NO: 180 - DNA, 21 bp, synthetic construct
  misc_feature 1..21: note = B2M gRNA 3
  source 1..21: mol_type = other DNA
cgctactctctctttctggcc

SEQ ID NO: 181 - DNA, 21 bp, synthetic construct
  misc_feature 1..21: note = B2M gRNA 4
  source 1..21: mol_type = other DNA
gcgcgagcacagctaaggcca

SEQ ID NO: 182 - DNA, 21 bp, synthetic construct
  misc_feature 1..21: note = B2M gRNA 6
  source 1..21: mol_type = other DNA
gctcgcgctactctctctttc

SEQ ID NO: 183 - DNA, 21 bp, synthetic construct
  misc_feature 1..21: note = B2M gRNA 7
  source 1..21: mol_type = other DNA
agagtagcgcgagcacagcta

SEQ ID NO: 184 - DNA, 21 bp, synthetic construct
  misc_feature 1..21: note = B2M gRNA 15
  source 1..21: mol_type = other DNA
tcacagcccaagatagttaag

SEQ ID NO: 185 - DNA, 21 bp, synthetic construct
  misc_feature 1..21: note = B2M gRNA 16
  source 1..21: mol_type = other DNA
cacagcccaagatagttaagt

SEQ ID NO: 186 - DNA, 21 bp, synthetic construct
  misc_feature 1..21: note = B2M gRNA 18
  source 1..21: mol_type = other DNA
gacaaagtcacatggttcaca

SEQ ID NO: 187 - DNA, 21 bp, synthetic construct
  misc_feature 1..21: note = B2M gRNA 19
  source 1..21: mol_type = other DNA
aagtcacatggttcacacggc

SEQ ID NO: 188 - DNA, 21 bp, synthetic construct
  misc_feature 1..21: note = B2M gRNA 20
  source 1..21: mol_type = other DNA
aggcatactcatctttttcag

SEQ ID NO: 189 - DNA, 21 bp, synthetic construct
  misc_feature 1..21: note = B2M gRNA 21
  source 1..21: mol_type = other DNA
ggcatactcatctttttcagt

SEQ ID NO: 190 - DNA, 21 bp, synthetic construct
  misc_feature 1..21: note = B2M gRNA 22
  source 1..21: mol_type = other DNA
catactcatctttttcagtgg

SEQ ID NO: 191 - DNA, 21 bp, synthetic construct
  misc_feature 1..21: note = B2M gRNA 23
  source 1..21: mol_type = other DNA
tcagtaagtcaacttcaatgt

SEQ ID NO: 192 - DNA, 21 bp, synthetic construct
  misc_feature 1..21: note = B2M gRNA 26
  source 1..21: mol_type = other DNA
acgtgagtaaacctgaatctt

SEQ ID NO: 193 - DNA, 20 bp, synthetic construct
  misc_feature 1..20: note = ELANEg35_OMNI-50
  source 1..20: mol_type = other DNA
agtccgggctgggagcgggt

SEQ ID NO: 194 - DNA, 20 bp, synthetic construct
  misc_feature 1..20: note = ELANEg38_OMNI-50
  source 1..20: mol_type = other DNA
acagcgggtgtagactccga

SEQ ID NO: 195 - DNA, 20 bp, synthetic construct
  misc_feature 1..20: note = ELANEg39_OMNI-50
  source 1..20: mol_type = other DNA
cagcgggtgtagactccgag

SEQ ID NO: 196 - DNA, 20 bp, synthetic construct
  misc_feature 1..20: note = ELANEg58_OMNI-50
  source 1..20: mol_type = other DNA
gctgcgggaaagggattccc

SEQ ID NO: 197 - DNA, 20 bp, synthetic construct
  misc_feature 1..20: note = ELANEg62_OMNI-50
  source 1..20: mol_type = other DNA
gtcaagccccagaggccaca